SEQUENCE LISTING (29 sequences)

SEQ ID NO: 1 - DNA, 592 bp, synthetic construct
  source 1..592: mol_type = other DNA
tcctattgttaactttgaacttccaattcatttggagaagtggtttcctcgtcatgtaataaagaccaaacgggaagatattcgagagctcatcttgaaacttcatttacagcaacagaagggcagcagtagctagttaatctgtacaaacatgacacagatgttctctaagattactggaaagcctcttaccagcatttgtgttagccagctcacagagaagaaaataacttgcagtagttttataataagtcattggaacattatttaaaatatgtaggacacattattagaattgttgggatctcatagatggaatgggaatgggggtgatatagataaacttactagatataaattaaaattttataaatatttcatatttttctgagtaaatatgattggattatgcaacagcatatgtaatatgggaatgttttgtagataataaaacttacatgatctgtacttccacgtgactgggtgctgaggggagttaaagcctccctggtgccagccccagtgcttgtcaaatttgctgacaggtcacatcatattgtaattctattctttgcagctcaagcatgcagta

SEQ ID NO: 2 - DNA, 606 bp, synthetic construct
  source 1..606: mol_type = other DNA
agttaattagttaatttaatcctcacattagctctaccatgagtttactatttctattccattttatacgtaaggaaggagacaaagtaagtgatttttctatcaaggaaggaaatttgcaagagaatagtttcattacaaaaactaaatttgtacgtagctctgtattattgaaataggtagatatagtcagtctggactttttatgcttatacatcttagtccctaggaaaacccagaactaacagattcagaaaagttggaaaaatcagtgaattatatgtgaaacacattattcttagtggactgcttgttaaaggcaaggagagtgttagtaaagagcttaggtagattagaataaagaaattgtctctctccatctgctctaattagcttatctcaccagcttttatagcatgctggttatttcagaaaagaagtgagagctactttgaaaggacaaccatttttctttccgctaatttataatggttttgaagtggttgttcattctcaaacatagacttttaaatgttaggtctttcctataactctttgttattggaagtttcaaggatttggacactcaattaaggattctgtcct

SEQ ID NO: 3 - DNA, 1198 bp, synthetic construct
  source 1..1198: mol_type = other DNA
tcctattgttaactttgaacttccaattcatttggagaagtggtttcctcgtcatgtaataaagaccaaacgggaagatattcgagagctcatcttgaaacttcatttacagcaacagaagggcagcagtagctagttaatctgtacaaacatgacacagatgttctctaagattactggaaagcctcttaccagcatttgtgttagccagctcacagagaagaaaataacttgcagtagttttataataagtcattggaacattatttaaaatatgtaggacacattattagaattgttgggatctcatagatggaatgggaatgggggtgatatagataaacttactagatataaattaaaattttataaatatttcatatttttctgagtaaatatgattggattatgcaacagcatatgtaatatgggaatgttttgtagataataaaacttacatgatctgtacttccacgtgactgggtgctgaggggagttaaagcctccctggtgccagccccagtgcttgtcaaatttgctgacaggtcacatcatattgtaattctattctttgcagctcaagcatgcagtaagttaattagttaatttaatcctcacattagctctaccatgagtttactatttctattccattttatacgtaaggaaggagacaaagtaagtgatttttctatcaaggaaggaaatttgcaagagaatagtttcattacaaaaactaaatttgtacgtagctctgtattattgaaataggtagatatagtcagtctggactttttatgcttatacatcttagtccctaggaaaacccagaactaacagattcagaaaagttggaaaaatcagtgaattatatgtgaaacacattattcttagtggactgcttgttaaaggcaaggagagtgttagtaaagagcttaggtagattagaataaagaaattgtctctctccatctgctctaattagcttatctcaccagcttttatagcatgctggttatttcagaaaagaagtgagagctactttgaaaggacaaccatttttctttccgctaatttataatggttttgaagtggttgttcattctcaaacatagacttttaaatgttaggtctttcctataactctttgttattggaagtttcaaggatttggacactcaattaaggattctgtcct

SEQ ID NO: 4 - DNA, 302 bp, synthetic construct
  source 1..302: mol_type = other DNA
gactgcttgttaaaggcaaggagagtgttagtaaagagcttaggtagattagaataaagaaattgtctctctccatctgctctaattagcttatctcaccagcttttatagcatgctggttatttcagaaaagaagtgagagctactttgaaaggacaaccatttttctttccgctaatttataatggttttgaagtggttgttcattctcaaacatagacttttaaatgttaggtctttcctataactctttgttattggaagtttcaaggatttggacactcaattaaggattctgtcct

SEQ ID NO: 5 - DNA, 894 bp, synthetic construct
  source 1..894: mol_type = other DNA
tcctattgttaactttgaacttccaattcatttggagaagtggtttcctcgtcatgtaataaagaccaaacgggaagatattcgagagctcatcttgaaacttcatttacagcaacagaagggcagcagtagctagttaatctgtacaaacatgacacagatgttctctaagattactggaaagcctcttaccagcatttgtgttagccagctcacagagaagaaaataacttgcagtagttttataataagtcattggaacattatttaaaatatgtaggacacattattagaattgttgggatctcatagatggaatgggaatgggggtgatatagataaacttactagatataaattaaaattttataaatatttcatatttttctgagtaaatatgattggattatgcaacagcatatgtaatatgggaatgttttgtagataataaaacttacatgatctgtacttccacgtgactgggtgctgaggggagttaaagcctccctggtgccagccccagtgcttgtcaaatttgctgacaggtcacatcatattgtaattctattctttgcagctcaagcatgcagtagactgcttgttaaaggcaaggagagtgttagtaaagagcttaggtagattagaataaagaaattgtctctctccatctgctctaattagcttatctcaccagcttttatagcatgctggttatttcagaaaagaagtgagagctactttgaaaggacaaccatttttctttccgctaatttataatggttttgaagtggttgttcattctcaaacatagacttttaaatgttaggtctttcctataactctttgttattggaagtttcaaggatttggacactcaattaaggattctgtcct

SEQ ID NO: 6 - DNA, 300 bp, synthetic construct
  source 1..300: mol_type = other DNA
tcctattgttaactttgaacttccaattcatttggagaagtggtttcctcgtcatgtaataaagaccaaacgggaagatattcgagagctcatcttgaaacttcatttacagcaacagaagggcagcagtagctagttaatctgtacaaacatgacacagatgttctctaagattactggaaagcctcttaccagcatttgtgttagccagctcacagagaagaaaataacttgcagtagttttataataagtcattggaacattatttaaaatatgtaggacacattattagaattgtt

SEQ ID NO: 7 - DNA, 906 bp, synthetic construct
  source 1..906: mol_type = other DNA
tcctattgttaactttgaacttccaattcatttggagaagtggtttcctcgtcatgtaataaagaccaaacgggaagatattcgagagctcatcttgaaacttcatttacagcaacagaagggcagcagtagctagttaatctgtacaaacatgacacagatgttctctaagattactggaaagcctcttaccagcatttgtgttagccagctcacagagaagaaaataacttgcagtagttttataataagtcattggaacattatttaaaatatgtaggacacattattagaattgttagttaattagttaatttaatcctcacattagctctaccatgagtttactatttctattccattttatacgtaaggaaggagacaaagtaagtgatttttctatcaaggaaggaaatttgcaagagaatagtttcattacaaaaactaaatttgtacgtagctctgtattattgaaataggtagatatagtcagtctggactttttatgcttatacatcttagtccctaggaaaacccagaactaacagattcagaaaagttggaaaaatcagtgaattatatgtgaaacacattattcttagtggactgcttgttaaaggcaaggagagtgttagtaaagagcttaggtagattagaataaagaaattgtctctctccatctgctctaattagcttatctcaccagcttttatagcatgctggttatttcagaaaagaagtgagagctactttgaaaggacaaccatttttctttccgctaatttataatggttttgaagtggttgttcattctcaaacatagacttttaaatgttaggtctttcctataactctttgttattggaagtttcaaggatttggacactcaattaaggattctgtcct

SEQ ID NO: 8 - DNA, 602 bp, synthetic construct
  source 1..602: mol_type = other DNA
tcctattgttaactttgaacttccaattcatttggagaagtggtttcctcgtcatgtaataaagaccaaacgggaagatattcgagagctcatcttgaaacttcatttacagcaacagaagggcagcagtagctagttaatctgtacaaacatgacacagatgttctctaagattactggaaagcctcttaccagcatttgtgttagccagctcacagagaagaaaataacttgcagtagttttataataagtcattggaacattatttaaaatatgtaggacacattattagaattgttgactgcttgttaaaggcaaggagagtgttagtaaagagcttaggtagattagaataaagaaattgtctctctccatctgctctaattagcttatctcaccagcttttatagcatgctggttatttcagaaaagaagtgagagctactttgaaaggacaaccatttttctttccgctaatttataatggttttgaagtggttgttcattctcaaacatagacttttaaatgttaggtctttcctataactctttgttattggaagtttcaaggatttggacactcaattaaggattctgtcct

SEQ ID NO: 9 - DNA, 2382 bp, synthetic construct
  source 1..2382: mol_type = other DNA
tcctattgttaactttgaacttccaattcatttggagaagtggtttcctcgtcatgtaataaagaccaaacgggaagatattcgagagctcatcttgaaacttcatttacagcaacagaagggcagcagtagctagttaatctgtacaaacatgacacagatgttctctaagattactggaaagcctcttaccagcatttgtgttagccagctcacagagaagaaaataacttgcagtagttttataataagtcattggaacattatttaaaatatgtaggacacattattagaattgttgggatctcatagatggaatgggaatgggggtgatatagataaacttactagatataaattaaaattttataaatatttcatatttttctgagtaaatatgattggattatgcaacagcatatgtaatatgggaatgttttgtagataataaaacttacatgatctgtacttccacgtgactgggtgctgaggggagttaaagcctccctggtgccagccccagtgcttgtcaaatttgctgacaggtcacatcatattgtaattctattctttgcagctcaagcatgcagtatcctattgttaactttgaacttccaattcatttggagaagtggtttcctcgtcatgtaataaagaccaaacgggaagatattcgagagctcatcttgaaacttcatttacagcaacagaagggcagcagtagctagttaatctgtacaaacatgacacagatgttctctaagattactggaaagcctcttaccagcatttgtgttagccagctcacagagaagaaaataacttgcagtagttttataataagtcattggaacattatttaaaatatgtaggacacattattagaattgttgggatctcatagatggaatgggaatgggggtgatatagataaacttactagatataaattaaaattttataaatatttcatatttttctgagtaaatatgattggattatgcaacagcatatgtaatatgggaatgttttgtagataataaaacttacatgatctgtacttccacgtgactgggtgctgaggggagttaaagcctccctggtgccagccccagtgcttgtcaaatttgctgacaggtcacatcatattgtaattctattctttgcagctcaagcatgcagtatcctattgttaactttgaacttccaattcatttggagaagtggtttcctcgtcatgtaataaagaccaaacgggaagatattcgagagctcatcttgaaacttcatttacagcaacagaagggcagcagtagctagttaatctgtacaaacatgacacagatgttctctaagattactggaaagcctcttaccagcatttgtgttagccagctcacagagaagaaaataacttgcagtagttttataataagtcattggaacattatttaaaatatgtaggacacattattagaattgttgggatctcatagatggaatgggaatgggggtgatatagataaacttactagatataaattaaaattttataaatatttcatatttttctgagtaaatatgattggattatgcaacagcatatgtaatatgggaatgttttgtagataataaaacttacatgatctgtacttccacgtgactgggtgctgaggggagttaaagcctccctggtgccagccccagtgcttgtcaaatttgctgacaggtcacatcatattgtaattctattctttgcagctcaagcatgcagtaagttaattagttaatttaatcctcacattagctctaccatgagtttactatttctattccattttatacgtaaggaaggagacaaagtaagtgatttttctatcaaggaaggaaatttgcaagagaatagtttcattacaaaaactaaatttgtacgtagctctgtattattgaaataggtagatatagtcagtctggactttttatgcttatacatcttagtccctaggaaaacccagaactaacagattcagaaaagttggaaaaatcagtgaattatatgtgaaacacattattcttagtggactgcttgttaaaggcaaggagagtgttagtaaagagcttaggtagattagaataaagaaattgtctctctccatctgctctaattagcttatctcaccagcttttatagcatgctggttatttcagaaaagaagtgagagctactttgaaaggacaaccatttttctttccgctaatttataatggttttgaagtggttgttcattctcaaacatagacttttaaatgttaggtctttcctataactctttgttattggaagtttcaaggatttggacactcaattaaggattctgtcct

SEQ ID NO: 10 - DNA, 50 bp, synthetic construct
  source 1..50: mol_type = other DNA
taggcgtgtacggtgggaggtctatataagcagagctcgtttagtgaacc

SEQ ID NO: 11 - DNA, 642 bp, synthetic construct
  source 1..642: mol_type = other DNA
tcctattgttaactttgaacttccaattcatttggagaagtggtttcctcgtcatgtaataaagaccaaacgggaagatattcgagagctcatcttgaaacttcatttacagcaacagaagggcagcagtagctagttaatctgtacaaacatgacacagatgttctctaagattactggaaagcctcttaccagcatttgtgttagccagctcacagagaagaaaataacttgcagtagttttataataagtcattggaacattatttaaaatatgtaggacacattattagaattgttgggatctcatagatggaatgggaatgggggtgatatagataaacttactagatataaattaaaattttataaatatttcatatttttctgagtaaatatgattggattatgcaacagcatatgtaatatgggaatgttttgtagataataaaacttacatgatctgtacttccacgtgactgggtgctgaggggagttaaagcctccctggtgccagccccagtgcttgtcaaatttgctgacaggtcacatcatattgtaattctattctttgcagctcaagcatgcagtataggcgtgtacggtgggaggtctatataagcagagctcgtttagtgaacc

SEQ ID NO: 12 - DNA, 295 bp, synthetic construct
  source 1..295: mol_type = other DNA
gggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccgggctcccaggggcttcccagtggtccccagggaaccctcgacagggccagggcgtctctctcgtccagcaagggcagggacgggccacaggcaagggc

SEQ ID NO: 13 - DNA, 1733 bp, synthetic construct
  source 1..1733: mol_type = other DNA
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattg

SEQ ID NO: 14 - DNA, 370 bp, synthetic construct
  source 1..370: mol_type = other DNA
tattattgaaataggtagatatagtcagtctggactttttatgcttatacatcttagtccctaggaaaacccagaactaacagattcagaaaagttggaaaaatcagtgaattatatgtgaaacacattattcttagtggactgcttgttaaaggcaaggagagtgttagtaaagagcttaggtagattagaataaagaaattgtctctctccatctgctctaattagcttatctcaccagcttttatagcatgctggttatttcagaaaagaagtgagagctactttgaaaggacaaccatttttctttccgctaatttataatggttttgaagtggttgttcattctcaaacatagacttttaaatgt

SEQ ID NO: 15 - DNA, 670 bp, synthetic construct
  source 1..670: mol_type = other DNA
tcctattgttaactttgaacttccaattcatttggagaagtggtttcctcgtcatgtaataaagaccaaacgggaagatattcgagagctcatcttgaaacttcatttacagcaacagaagggcagcagtagctagttaatctgtacaaacatgacacagatgttctctaagattactggaaagcctcttaccagcatttgtgttagccagctcacagagaagaaaataacttgcagtagttttataataagtcattggaacattatttaaaatatgtaggacacattattagaattgtttattattgaaataggtagatatagtcagtctggactttttatgcttatacatcttagtccctaggaaaacccagaactaacagattcagaaaagttggaaaaatcagtgaattatatgtgaaacacattattcttagtggactgcttgttaaaggcaaggagagtgttagtaaagagcttaggtagattagaataaagaaattgtctctctccatctgctctaattagcttatctcaccagcttttatagcatgctggttatttcagaaaagaagtgagagctactttgaaaggacaaccatttttctttccgctaatttataatggttttgaagtggttgttcattctcaaacatagacttttaaatgt

SEQ ID NO: 16 - DNA, 360 bp, synthetic construct
  source 1..360: mol_type = other DNA
agaaattaagctcttttaatggatgtttcttgtacatgtcattttagaaaacatctgaccctaactgtcagccttattctctgtttggcagaacttcccctggctctctgtgtcactgtaacaggtgaataactaagaaaaaactgtgtctgtagacacttgtttataatggcattcagggtcctggagctaggctgacagatgctcctccagaaggttaatgagataaaggttcctccagctggcccttaagcagagattacacctgagggaaagacaagcagattattccagaaacagacactgctacatgttcttcataaattaacaccctcataaaggtaaaccaagaaggttatc

SEQ ID NO: 17 - DNA, 730 bp, synthetic construct
  source 1..730: mol_type = other DNA
gtggaccctaacacactgcaggagtggtatttgtatcctgctaactctagctgaagtgctagcacctcaccagtgccagaaaaccagcacacagcagctcaggccaggaggagaggcttgcaccatgccacagagctctgcagcccctcacagactacctggaagctggcttggttctgtttcctttcacacttgctggcactgaaacagttaggacaaacctattgagcgctattctctgatcatccctcagtagtaaacagctctaatgacaaggagaacagccctgcttgaataaacttttaaatgcttttcaaaattaagccactgtgactattttaacaaaaatcttaaatcttaaaaatatatcactcagttatcatgtgtggaaaattgagaacgttttttgagaaaggttactgtaagacttaaggaagaaagacctaaatccccactgtgatggtatttggaaggtaattaggtcaaaggtaagagcctttgggatgtaattaggtcatgagggccgagcccttataagaagagacaaaagagggatgatttctctctccctttctctctctctctttctttctctctctctccctttctctccctttctctctctctttctctctctccttttccgccatgtggggatacaactagaggacagctgcctacaaactaagaagagtgacctcactagacactggtttttctggtgccttga

SEQ ID NO: 18 - DNA, 1089 bp, synthetic construct
  source 1..1089: mol_type = other DNA
gtggaccctaacacactgcaggagtggtatttgtatcctgctaactctagctgaagtgctagcacctcaccagtgccagaaaaccagcacacagcagctcaggccaggaggagaggcttgcaccatgccacagagctctgcagcccctcacagactacctggaagctggcttggttctgtttcctttcacacttgctggcactgaaacagttaggacaaacctattgagcgctattctctgatcatccctcagtagtaaacagctctaatgacaaggagaacagccctgcttgaataaacttttaaatgcttttcaaaattaagccactgtgactattttaacaaaaatcttaaatcttaaaaatatatcactcagttatcatgtgtggaaaattgagaacgttttttgagaaaggttactgtaagacttaaggaagaaagacctaaatccccactgtgatggtatttggaaggtaattaggtcaaaggtaagagcctttgggatgtaattaggtcatgagggccgagcccttataagaagagacaaaagagggatgatttctctctccctttctctctctctctttctttctctctctctccctttctctccctttctctctctctttctctctctccttttccgccatgtggggatacaactagaggacagctgcctacaaactaagaagagtgacctcactagacactggtttttctggtgccttgaagaaattaagctcttttaatggatgtttcttgtacatgtcattttagaaaacatctgaccctaactgtcagccttattctctgtttggcagaacttcccctggctctctgtgtcactgtaacaggtgaataactaagaaaaaactgtgtctgtagacacttgtttataatggcattcagggtcctggagctaggctgacagatgctcctccagaaggttaatgagataaaggttcctccagctggcccttaagcagagattacacctgagggaaagacaagcaattattccagaaacagacactgctacatgttcttcataaattaacaccctcataaaggtaaaccaagaaggttatc

SEQ ID NO: 19 - DNA, 952 bp, synthetic construct
  source 1..952: mol_type = other DNA
tcctattgttaactttgaacttccaattcatttggagaagtggtttcctcgtcatgtaataaagaccaaacgggaagatattcgagagctcatcttgaaacttcatttacagcaacagaagggcagcagtagctagttaatctgtacaaacatgacacagatgttctctaagattactggaaagcctcttaccagcatttgtgttagccagctcacagagaagaaaataacttgcagtagttttataataagtcattggaacattatttaaaatatgtaggacacattattagaattgttgggatctcatagatggaatgggaatgggggtgatatagataaacttactagatataaattaaaattttataaatatttcatatttttctgagtaaatatgattggattatgcaacagcatatgtaatatgggaatgttttgtagataataaaacttacatgatctgtacttccacgtgactgggtgctgaggggagttaaagcctccctggtgccagccccagtgcttgtcaaatttgctgacaggtcacatcatattgtaattctattctttgcagctcaagcatgcagtaagaaattaagctcttttaatggatgtttcttgtacatgtcattttagaaaacatctgaccctaactgtcagccttattctctgtttggcagaacttcccctggctctctgtgtcactgtaacaggtgaataactaagaaaaaactgtgtctgtagacacttgtttataatggcattcagggtcctggagctaggctgacagatgctcctccagaaggttaatgagataaaggttcctccagctggcccttaagcagagattacacctgagggaaagacaagcagattattccagaaacagacactgctacatgttcttcataaattaacaccctcataaaggtaaaccaagaaggttatc

SEQ ID NO: 20 - DNA, 660 bp, synthetic construct
  source 1..660: mol_type = other DNA
tcctattgttaactttgaacttccaattcatttggagaagtggtttcctcgtcatgtaataaagaccaaacgggaagatattcgagagctcatcttgaaacttcatttacagcaacagaagggcagcagtagctagttaatctgtacaaacatgacacagatgttctctaagattactggaaagcctcttaccagcatttgtgttagccagctcacagagaagaaaataacttgcagtagttttataataagtcattggaacattatttaaaatatgtaggacacattattagaattgttagaaattaagctcttttaatggatgtttcttgtacatgtcattttagaaaacatctgaccctaactgtcagccttattctctgtttggcagaacttcccctggctctctgtgtcactgtaacaggtgaataactaagaaaaaactgtgtctgtagacacttgtttataatggcattcagggtcctggagctaggctgacagatgctcctccagaaggttaatgagataaaggttcctccagctggcccttaagcagagattacacctgagggaaagacaagcagattattccagaaacagacactgctacatgttcttcataaattaacaccctcataaaggtaaaccaagaaggttatc

SEQ ID NO: 21 - DNA, 589 bp, synthetic construct
  source 1..589: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatc

SEQ ID NO: 22 - DNA, 1948 bp, synthetic construct
  source 1..1948: mol_type = other DNA
tcctattgttaactttgaacttccaattcatttggagaagtggtttcctcgtcatgtaataaagaccaaacgggaagatattcgagagctcatcttgaaacttcatttacagcaacagaagggcagcagtagctagttaatctgtacaaacatgacacagatgttctctaagattactggaaagcctcttaccagcatttgtgttagccagctcacagagaagaaaataacttgcagtagttttataataagtcattggaacattatttaaaatatgtaggacacattattagaattgttgggatctcatagatggaatgggaatgggggtgatatagataaacttactagatataaattaaaattttataaatatttcatatttttctgagtaaatatgattggattatgcaacagcatatgtaatatgggaatgttttgtagataataaaacttacatgatctgtacttccacgtgactgggtgctgaggggagttaaagcctccctggtgccagccccagtgcttgtcaaatttgctgacaggtcacatcatattgtaattctattctttgcagctcaagcatgcagtaagttaattagttaatttaatcctcacattagctctaccatgagtttactatttctattccattttatacgtaaggaaggagacaaagtaagtgatttttctatcaaggaaggaaatttgcaagagaatagtttcattacaaaaactaaatttgtacgtagctctgtattattgaaataggtagatatagtcagtctggactttttatgcttatacatcttagtccctaggaaaacccagaactaacagattcagaaaagttggaaaaatcagtgaattatatgtgaaacacattattcttagtggactgcttgttaaaggcaaggagagtgttagtaaagagcttaggtagattagaataaagaaattgtctctctccatctgctctaattagcttatctcaccagcttttatagcatgctggttatttcagaaaagaagtgagagctactttgaaaggacaaccatttttctttccgctaatttataatggttttgaagtggttgttcattctcaaacatagacttttaaatgttaggtctttcctataactctttgttattggaagtttcaaggatttggacactcaattaaggattctgtcctcaagtttgtacaaaaaagcaggctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 23 - DNA, 608 bp, synthetic construct
  source 1..608: mol_type = other DNA
aaaacgtgctttttccacggaactgtgcaacccacagatcagaagatctcacttgggagcccatacctccggggcctagggtcccaaccacagaaccaagcagattttcaacagccactaagctagaatctgcttaagcctgcggagctccccgcgggaggggcaaccagcaccaccgctgtggctgcctgctgtctaagccctttgaactcctttgcaggaggggctacagccaacactgggactgatagctgcctaacacactaagctcccagggtgggggaagggtggcagccatctctatagctccaggccatgtctttcccctgctggagccagggaggctggacggcttgctcccaagaggtataccccacagtccaacacactagctgtggcagactgcggacagagcgcctcttcaggccggaccttgacatgtccctgctcactgggtggggcctccttgcaggaactccaacaactccatccagggacttagggacagactctaatcttcctgggcttgagcctctagagggaggggtggccacagtctctgcggacgggtagatttagtcttccctcctggtagtgctgaggaatct

SEQ ID NO: 24 - DNA, 1214 bp, synthetic construct
  source 1..1214: mol_type = other DNA
aaaacgtgctttttccacggaactgtgcaacccacagatcagaagatctcacttgggagcccatacctccggggcctagggtcccaaccacagaaccaagcagattttcaacagccactaagctagaatctgcttaagcctgcggagctccccgcgggaggggcaaccagcaccaccgctgtggctgcctgctgtctaagccctttgaactcctttgcaggaggggctacagccaacactgggactgatagctgcctaacacactaagctcccagggtgggggaagggtggcagccatctctatagctccaggccatgtctttcccctgctggagccagggaggctggacggcttgctcccaagaggtataccccacagtccaacacactagctgtggcagactgcggacagagcgcctcttcaggccggaccttgacatgtccctgctcactgggtggggcctccttgcaggaactccaacaactccatccagggacttagggacagactctaatcttcctgggcttgagcctctagagggaggggtggccacagtctctgcggacgggtagatttagtcttccctcctggtagtgctgaggaatctagttaattagttaatttaatcctcacattagctctaccatgagtttactatttctattccattttatacgtaaggaaggagacaaagtaagtgatttttctatcaaggaaggaaatttgcaagagaatagtttcattacaaaaactaaatttgtacgtagctctgtattattgaaataggtagatatagtcagtctggactttttatgcttatacatcttagtccctaggaaaacccagaactaacagattcagaaaagttggaaaaatcagtgaattatatgtgaaacacattattcttagtggactgcttgttaaaggcaaggagagtgttagtaaagagcttaggtagattagaataaagaaattgtctctctccatctgctctaattagcttatctcaccagcttttatagcatgctggttatttcagaaaagaagtgagagctactttgaaaggacaaccatttttctttccgctaatttataatggttttgaagtggttgttcattctcaaacatagacttttaaatgttaggtctttcctataactctttgttattggaagtttcaaggatttggacactcaattaaggattctgtcct

SEQ ID NO: 25 - DNA, 747 bp, synthetic construct
  source 1..747: mol_type = other DNA
gattgaacccctctgcttacaacatctgttccctccccaggctccctagcagggccccaaccacctcacagcctgccatcagataactaatcagccccaccacagcccaatgcctgcattagcacattcaggcctaatcaggaattaatggacagtgcttcgcagtaatttaatttccacaataaacctaatgagcaatttaatacaaatgtcaaaaggaatcatttagttgttgtctgaatgctttctcctgtgcaaaccttcatgaaaactcaacacagctgcctttttatagcccagttaacatacattccagcagaatcagggaggcccctgtgcatttagtattattaataaggttaactgggcacttaattagcaatttagtggttagtgctccatgactagaaggtccccagaagattatttagcagaattaactgaggcttttgtcatatcctccctagatgccttcctcacactcctaaacaaagctgactcttaatcaatcccagataggttcacaaagagggcatggacataaacaataaatcccttccacccaagccaatgccatggagccctcctggagaggaggagggctctcccccgcagtgctccctgttgctgatgctgcagctgcaggcatctacaatgccagtcccagggtcgattgactgttttcatgtatataacagcatctctggtatcctggagaagaaaaatggatgattcaggatctgaatc

SEQ ID NO: 26 - DNA, 399 bp, synthetic construct
  source 1..399: mol_type = other DNA
acagaaattaagctcttttaatggatgtttcttgtacatgtcattttagaaaacatctgaccctaactgtcagccttattctctgtttggcagaacttcccctggctctctgtgtcactgtaacaggtgaataactaagaaaaaactgtgtctgtagacacttgtttataatggcattcagggtcctggagctaggctgacagatgctcctccagaaggttaatgagataaaggttcctccagctggcccttaagcagagattacacctgagggaaagacaagcagattattccagaaacagacactgctacatgttcttcataaattaacaccctcataaaggtaaaccaagaaggttatcctcaatcatctggtatcaatatataattatttttcac

SEQ ID NO: 27 - DNA, 1146 bp, synthetic construct
  source 1..1146: mol_type = other DNA
gattgaacccctctgcttacaacatctgttccctccccaggctccctagcagggccccaaccacctcacagcctgccatcagataactaatcagccccaccacagcccaatgcctgcattagcacattcaggcctaatcaggaattaatggacagtgcttcgcagtaatttaatttccacaataaacctaatgagcaatttaatacaaatgtcaaaaggaatcatttagttgttgtctgaatgctttctcctgtgcaaaccttcatgaaaactcaacacagctgcctttttatagcccagttaacatacattccagcagaatcagggaggcccctgtgcatttagtattattaataaggttaactgggcacttaattagcaatttagtggttagtgctccatgactagaaggtccccagaagattatttagcagaattaactgaggcttttgtcatatcctccctagatgccttcctcacactcctaaacaaagctgactcttaatcaatcccagataggttcacaaagagggcatggacataaacaataaatcccttccacccaagccaatgccatggagccctcctggagaggaggagggctctcccccgcagtgctccctgttgctgatgctgcagctgcaggcatctacaatgccagtcccagggtcgattgactgttttcatgtatataacagcatctctggtatcctggagaagaaaaatggatgattcaggatctgaatcacagaaattaagctcttttaatggatgtttcttgtacatgtcattttagaaaacatctgaccctaactgtcagccttattctctgtttggcagaacttcccctggctctctgtgtcactgtaacaggtgaataactaagaaaaaactgtgtctgtagacacttgtttataatggcattcagggtcctggagctaggctgacagatgctcctccagaaggttaatgagataaaggttcctccagctggcccttaagcagagattacacctgagggaaagacaagcagattattccagaaacagacactgctacatgttcttcataaattaacaccctcataaaggtaaaccaagaaggttatcctcaatcatctggtatcaatatataattatttttcac

SEQ ID NO: 28 - DNA, 724 bp, synthetic construct
  source 1..724: mol_type = other DNA
tttggcaagtaagtttcttataagcttaccagtattttgcaaatacaactatgcaaatatatttaatggtcatttaggtttattagcttttataaaggctgaaaatgtggtttatttgaggctgtattgaaaaaatatacttgagcttttcctaaagcataaaataacattgagggtgatttaagaggaatgtggtttagatctttacaatacactttttttcagagaattttgccagagataacatgaaataaaatataatttcattgctatttgatagtaaatctttacttgatttaaaaaactgttctaatatagagaatttcatctgcagggaaaatgttttcttggttaagagttcctttaagcaacaaagttatcgcacagctatcatttatattaagtgcttaatatgttctaagcaaagtgaagattgaattagttaattagttaatttaatcctcacattagctctaccatactatttctattccattttatacgtaaggaaggagacaaaggaaatttgcaagagaatagtttcattacaaaaactaaatttgtacgtagctctgtattattgaaataggtagatatagtcagtctggactttttatgcttatatggaaaaatcagtgaattatatgtgaaacacattattcttagttagaataaagaaattgtctctctccatctgctctaattagcttatctcaccagcttt

SEQ ID NO: 29 - DNA, 783 bp, synthetic construct
  source 1..783: mol_type = other DNA
tttggcaagtaagtttcttataagcttaccagtattttgcaaatacaactatgcaaatatatttaatggtcatttaggtttattagcttttataaaggctgaaaatgtggtttatttgaggctgtattgaaaaaatatacttgagcttttcctaaagcataaaataacattgagggtgatttaagaggaatgtggtttagatctttacaatacactttttttcagagaattttgccagagataacatgaaataaaatataatttcattgctatttgatagtaaatctttacttgatttaaaaaactgttctaatatagagaatttcatctgcagggaaaatgttttcttggttaagagttcctttaagcaacaaagttatcgcacagctatcatttatattaagtgcttaatatgttctaagcaaagtgaagattgaattagttaattagttaatttaatcctcacattagctctaccatactatttctattccattttatacgtaaggaaggagacaaaggaaatttgcaagagaatagtttcattacaaaaactaaatttgtacgtagctctgtattattgaaataggtagatatagtcagtctggactttttatgcttatatggaaaaatcagtgaattatatgtgaaacacattattcttagttagaataaagaaattgtctctctccatctgctctaattagcttatctcaccagcttttaggcgtgtacggtgggaggtctatataagcagagctcgtttagtgaaccgccaccatg

(19) United States
(12) Patent Application Publication
(10) Pub. No.: US 2026/0007776 A1
(43) Pub. Date: Jan. 8, 2026

(54) DESIGN AND APPLICATION OF NOVEL PHOTORECEPTOR-SPECIFIC PROMOTERS FOR RETINAL GENE THERAPY APPLICATIONS

(71) Applicant: Wisconsin Alumni Research Foundation, Madision, WI (US)

(72) Inventors: David Gamm, Madison, WI (US); Yashdeep Phanse, Madison, WI (US)

(21) Appl. No.: 19/261,595

(22) Filed: Jul. 7, 2025

Related U.S. Application Data
(60) Provisional application No. 63/668,131, filed on Jul. 5, 2024.

(51) Int. Cl. A61K 48/00; A61K 9/00; A61P 27/02; C12N 15/113; C12N 15/86

(52) U.S. Cl. CPC A61K 48/0058; A61K 9/0048; A61K 48/0075; A61P 27/02; C12N 15/113; C12N 15/86; C12N 2310/14; C12N 2750/14143

(57) ABSTRACT

Provided herein are genetically engineered promoter-enhancer constructs for altering transcription of a gene operably linked thereto having specificity for expression in photoreceptor cells. Also provided are methods of use for these photoreceptor-specific promoter-enhancer genetic constructs; a pharmaceutical composition comprising these constructs, and methods of using the pharmaceutical composition for treating retinal diseases.

### CROSS-REFERENCE TO RELATED APPLICATIONS

This application claims priority to U.S. provisional application No. 63/668,131, filed Jul. 5, 2024, the disclosure of which is expressly incorporated by reference herein.

### REFERENCE TO AN ELECTRONIC SEQUENCE LISTING

This application contains a Sequence Listing that has been submitted electronically and is hereby incorporated by reference in its entirety. The Sequence Listing was created on Jul. 1, 2025, is named “23-1638-US_SequenceListing.xml”, and is 49,375 bytes in size.

### BACKGROUND OF THE DISCLOSURE

Effective treatment of outer neuroretinal disease poses a significant challenge, necessitating development of therapeutic components with precise control over gene expression in target photoreceptors. An ideal promoter for such applications should exhibit satisfactory expression levels in the desired photoreceptor cells while minimizing or preventing expression in off-target (i.e., non-photoreceptor) cells. Currently, available promoters, despite being widely used and sought after, often fall short in meeting these criteria. Many existing promoters suffer from inherent deficiencies, leading to the use of nonspecific promoters such as phosphoglycerol kinase (PGK), cytomegalovirus (CMV), or CMV early enhancer/chicken β actin (CAG) promoters. This compromise is made with the hope that expression in bystander cells will not have detrimental effects.

These limitations of existing promoters highlight the urgent need for a novel promoter that can specifically drive satisfactory expression levels for genes of interest in target photoreceptor cells while maintaining minimal expression in off-target cells. Such a promoter would be useful in the treatment of various outer retinal diseases, including retinitis pigmentosa, Leber congenital amaurosis, Usher syndrome, Stargardt disease, and other macular degeneration disorders. However, the relative lack of robust yet specific photoreceptor promoters for in vitro and in vivo model testing and human clinical trials has limited the development of these therapies from both safety and efficacy standpoints.

### SUMMARY OF THE DISCLOSURE

The invention disclosed herein provides genetically engineered, photoreceptor-specific promoter-enhancer constructs that exhibit beneficial expression levels in the desired photoreceptor cells while minimizing or preventing expression in off-target cells.

Also provided herein are methods for introducing novel genetically engineered, photoreceptor-specific promoter-enhancer constructs into target photoreceptor cells. These constructs and methods provide avenues for treating outer retinal diseases using gene therapeutic approaches.

Further provided herein are genetically engineered promoter-enhancer constructs comprising a promoter and at least one enhancer operatively linked to the promoter, wherein the enhancer specifically promotes expression in a photoreceptor cell of a gene that is operatively linked to the promoter-enhancer construct and capable of being expressed therein. The promoter-enhancer is positioned upstream of the nucleic acid. In specific embodiments, the genetically engineered promoter-enhancer construct comprises one or a plurality of enhancers derived from a human interphotoreceptor matrix proteoglycan (IMPG; either IMPG2 or IMPG1) gene having a nucleotide sequence that is at least 90% identical to SEQ ID NO: 1, SEQ ID NO: 6, or SEQ ID NO: 17. In some embodiments, the genetically engineered promoter-enhancer construct comprises one or a plurality of enhancers derived from a human IMPG (either IMPG2 or IMPG1) gene having a nucleotide sequence that is at least 90% identical to SEQ ID NO: 25. In specific embodiments, the genetically engineered promoter-enhancer construct comprises a promoter derived from a human IMPG (either IMPG2 or IMPG1) gene having a nucleotide sequence that is at least 90% identical to SEQ ID NO: 2, SEQ ID NO: 4, or SEQ ID NO: 7. In specific embodiments, the genetically engineered promoter-enhancer construct comprises a promoter derived from a human IMPG (either IMPG2 or IMPG1) gene having a nucleotide sequence that is at least 90% identical to SEQ ID NO: 26. In certain embodiments, the enhancer is positioned upstream from the promoter (as understood in the art equivalent to a position 5′ from the promoter) in the topography of the genetically engineered promoter-enhancer construct. In certain embodiments, the enhancer is positioned downstream stream from the promoter (as understood in the art equivalent to a position 3′ from the promoter) in the topography of the genetically engineered promoter-enhancer construct. In particular embodiments, the genetically engineered promoter-enhancer construct has a nucleotide sequence that is 90% identical to SEQ ID NO: 3, SEQ ID NO: 7, SEQ ID NO: 8, SEQ ID NO: 9, SEQ ID NO: 18, SEQ ID NO: 19, or SEQ ID NO: 20. In particular embodiments, the genetically engineered promoter-enhancer construct has a nucleotide sequence that is 90% identical to SEQ ID NO: 27.

In certain embodiments, the enhancer is positioned upstream from the native promoter in the topography of the IMPG1 and/or IMPG2 gene. In certain embodiments, the enhancer is positioned downstream from native promoter in the topography of the IMPG1 and/or IMPG2 gene.

Also provided herein are recombinant expression constructs capable of expressing a gene or a gene expression-modulating sequence in a photoreceptor cell comprising the promoter-enhancer construct disclosed herein, operatively linked to a nucleic acid encoding the gene or the gene expression-modulating sequence. In certain embodiments the recombinant expression construct further comprises a vector.

This disclosure also provides methods for modulating expression of a target gene in a photoreceptor cell comprising introducing into the cell a recombinant expression construct as disclosed herein. In certain embodiments, the gene expression-modulating sequence comprises an siRNA complementary to the target gene, the expression of which in the photoreceptor cell is modulated thereby. In alternative embodiments, the gene expression-modulating sequence comprises components of a gene editing system, wherein expression of the target gene in the photoreceptor cell is modulated thereby. In certain particular methods, expression of the encoded gene corrects a genetic mutation on the target gene in the photoreceptor cell.

This disclosure further provides a pharmaceutical composition comprising the disclosed recombinant expression construct and methods of treating a retinal disease in a subject comprising administering the pharmaceutical composition to the subject. In certain embodiments, the disease is a photoreceptor-specific or inherited retinal disease. Examples of such diseases include retinitis pigmentosa, Leber congenital amaurosis, Usher syndrome, Stargardt disease, and age-related or inherited macular degeneration. In certain embodiment, administration is performed by subretinal injection.

These and other features, objects, and advantages of the present invention will become better understood from the description that follows. In the description, reference is made to the accompanying drawings, which form a part hereof and in which there is shown by way of illustration, not limitation, embodiments of the invention. The description of preferred embodiments is not intended to limit the invention to cover all modifications, equivalents, and alternatives. Reference should therefore be made to the claims recited herein for interpreting the scope of the invention.

### BRIEF DESCRIPTION OF THE DRAWINGS

The patent or patent application file contains at least one drawing in color. Copies of this patent or patent application publication with color drawings will be provided by the Office upon request and payment of the necessary fec.

The disclosure will be better understood and features, aspects, and advantages other than those set forth above will become apparent when consideration is given to the following detailed description thereof. Such detailed description refers to the following figures.

FIGS. 1A-1B show graphs of single cell transcriptomics data in retinal cell types including photoreceptors (rods and cones). FIG. 1A shows IMPG2 gene measurements. FIG. 1B shows IMPG1 gene measurements.

FIG. 2 shows single-nucleus assays for transposase-accessible chromatin sequencing (snATAC-seq) data of the IMPG2 gene performed on developing and adult human retina and on induced plunpotent stem cell (iPSC)-derived retinal organoids.

FIG. 3 shows sequence alignment of peaks identified by snATAC-seq data with DNase I Hypersensitive site (DHS) data.

FIG. 4 shows sequence alignment of peaks identified by snATAC-seq data compared to Chromatin Immunoprecipitation Sequencing (ChIP-seq) data.

FIG. 5 shows immunocytochemistry characterization of Y79 human retinoblastoma cells using photoreceptor (PR)-specific antibody markers. Labeling in this Figure include EC-IMPG2: IMPG2 extracellular domain. IC-IMPG2: IMPG2 intracellular domain. CRX: Cone-Rod Homeobox. NRL: Neural Retina Leucine Zipper.

FIGS. 6A-6B show screening of promoter activity and specificity in human embryonic kidney cells (HEK) (negative control for photoreceptor specificity). FIG. 6A shows all promoters tested; FIG. 6B shows the same data with the CAG and CMV high outliers removed. Labeling in this Figure include GRK1: G Protein-Coupled Receptor Kinase 1 (SEQ ID NO: 12); IMPG2 P only: IMPG2 promoter only (SEQ ID NO: 2); IMPG2 PEI: IMPG2 Promoter IMPG2 Upstream Enhancer 1 (SEQ ID NO: 3); IMPG2 PE2: IMPG2 Promoter IMPG2 Downstream Enhancer 2 (SEQ ID NO: 24); IMPG1 P only: IMPG1 promoter only (SEQ ID NO: 26); IMPG1 PEI: IMPG1 Promoter IMPG1 Downstream Enhancer 1 (SEQ ID NO: 27); Synthetic IMPG2 Promoter: CMV core promoter+IMPG2 palindromic enhancer (SEQ ID NO: 29); Core promoter: CMV core promoter (SEQ ID NO: 10); CMV: Cytomegalovirus (CMV) full promoter (SEQ ID NO: 21); CAG: CMV early enhancer/chicken β actin promoter (SEQ ID NO: 13)

FIG. 7 shows screening of promoter activity and specificity in Y79 human retinoblastoma cells. Labeling in this Figure is the same as FIGS. 6A-6B.

FIG. 8 shows eGFP expression in the outer mantle of human pluripotent stem cell (hPSC)-derived retinal organoids 1-5 (RO1-RO5) at day 13 (D13) post transduction.

FIGS. 9A-9B show expression of eGFP and tdTomato in CRX17 ROs (a reporter line in which all PRs express red tdTomato fluorescence) by flow cytometry at D14 post-transduction, focusing on PRs. FIG. 9A shows transduction efficiency in PRs, FIG. 9B shows mean fluorescence intensity (MFI) of GFP⁺ PRs, and FIG. 9C shows integrated mean fluorescence intensity (iMFI) of GFP⁺ PRs (iMFI values were calculated as a product of percent transduction efficiency and MFI). *P<0.05, **P<0.01, ***P<0.001, ****P<0.0001, ns=not significant (P>0.05) (one-way ANOVA with Tukey analyses).

FIGS. 10A-10B show expression of eGFP and tdTomato by flow cytometry in CRX17 ROs at D14 post-transduction, focusing on non-PRs. FIG. 10A shows the % of total GFP⁺ cells that are GFP⁺ non-PR cells, and FIG. 10B shows MFI of GFP⁺ non-PRs. *P<0.05, **P<0.01, ***P<0.001, ****P<0.0001, ns=not significant (P>0.05) (one-way ANOVA with Tukey analyses).

FIG. 11 shows eGFP iMFI ratios of PRs versus eGFP iMFI of non-PRs. *P<0.05, **P<0.01, ***P<0.001, ****P<0.0001, ns=not significant (P>0.05) (one-way ANOVA with Tukey analyses).

FIG. 12 shows immunocytochemistry (ICC) analysis of the IMPG2 PE1-eGFP reporter construct 14 days post-transduction of human CRX17 ROs with AAV2.7m8 vector. Fluorescence from GFP expression strongly co-localized with the fluorescence from PR reporter TdTomato and the fluorescence from the PR marker recoverin.

FIGS. 13A-13D show ICC analysis of PR-specific eGFP expression 28 days after subretinal injection of AAV2.7m8 vector containing an IMPG2 PE1-eGFP reporter construct in mouse. FIGS. 13A-13B show brightfield and eGFP fluorescence; fluorescence from GFP expression driven by IMPG2-PE1 was within the PR region of the retina while fluorescence from GFP expression driven by CAG was found in all areas of the retina. FIGS. 13C-13D show ICC using antibodies directed against GFP (green) and the PR marker RECOVERIN (magenta). FIG. 13C: Fluorescence from GFP expression driven by IMPG2-PE1 mapped onto the fluorescence of Recoverin and location of PRs, while fluorescence from GFP expression driven by CAG was found in location of PRs and non-PRs, with some fluorescence that did not overlap with fluorescence of Recoverin. FIG. 13D: enlarged picture of a region of fluorescence from GFP expression driven by IMPG2-PE1, which mapped onto the fluorescence of recoverin. Bipolar cells are indicated by arrowheads.

FIG. 14 shows ICC analysis of the IMPG2 PEI-eGFP reporter construct post-transduction of human 10.13 iPSC-derived ROs with AAV2.7m8 vector. Fluorescence from GFP expression driven by IMPG2-PE1 (green) mapped onto fluorescence of Recoverin (red) in merged picture (GFP/Recoverin/DAPI) to produce orange color.

FIG. 15 shows ICC analysis of the IMPG2 PE1-eGFP reporter construct post-transduction of pig ROs with AAV2.7m8 vector. Fluorescence from GFP expression driven by IMPG2-PE1 (green) mapped onto fluorescence of Recoverin (red) in merged picture (GFP/Recoverin/DAPI) to produce orange color.

FIG. 16 shows a summary of the promoter/promoter-enhancer constructs that were tested in Round 1 screening.

FIG. 17 shows snATAC-seq data highlighting the full length and shortened promoter region of the IMPG2 gene.

FIG. 18 shows snATAC-seq data highlighting the full length and shortened enhancer region of the IMPG2 gene.

FIG. 19 shows eGFP expression in the outer mantle of retinal organoids (where photoreceptors largely reside in retinal organoids) 1-5 (RO1-RO5) transfected with AAV2.7m8 vector containing GFP under the regulation of Round 1 promoters/promoter-enhancer at day 14 (D14) post transduction.

FIG. 20 shows ICC analysis at 14 days post-transduction of human CRX17 ROs transfected with AAV2.7m8 vector containing GFP under the regulation of Round 1 promoters/promoter-enhancer. RCVRN: recoverin.

FIGS. 21A-21C show expression of eGFP and tdTomato in CRX17 ROs (a reporter line in which all PRs express red tdTomato fluorescence) with AAV2.7m8 vector containing GFP under the regulation of Round 1 promoters/promoter-enhancer, by flow cytometry at D14 post-transduction, focusing on PRs. FIG. 21A shows transduction efficiency in PRs, FIG. 21B shows mean fluorescence intensity (MFI) of GFP+PRs, and FIG. 21C shows integrated mean fluorescence intensity (IMFI) of GFP+PRs (iMFI values were calculated as a product of percent transduction efficiency and MFI). Tukey's multiple comparison test was performed and the statistical results are shown in Table 1-3.

FIGS. 22A-22C show expression of eGFP and tdTomato by flow cytometry in CRX17 ROs transfected with AAV2.7m8 vector containing GFP under the regulation of Round 1 promoters/promoter-enhancer, at D14 post-transduction, focusing on non-PRs. FIG. 22A shows the % of total GFP⁺ cells that are GFP⁺ non-PR cells. FIG. 22B shows MFI of GFP⁺ non-PRs. FIG. 22C shows integrated mean fluorescence intensity (iMFI) of GFP⁺ non-PRs (IMFI values were calculated as a product of percent transduction efficiency and MFI). Tukey's multiple comparison test was performed and the statistical results are shown in Table 4-6.

FIG. 23 shows eGFP integrated mean fluorescence intensity (iMFI) ratios of PRs versus eGFP iMFI of non-PRs in CRX17 ROs transfected with AAV2.7m8 vector containing GFP under the regulation of Round 1 promoters/promoter-enhancer. Tukey's multiple comparison test was performed and the statistical results are shown in Table 7.

FIG. 24 shows percentage of live cells in CRX17 ROs transfected with AAV2.7m8 vector containing GFP under the regulation of Round 1 promoters/promoter-enhancer. Tukey's multiple comparison test was performed and the statistical results are shown in Table 8.

FIG. 25 shows summary of the promoter/promoter-enhancer constructs that were tested in Round 2 screening.

FIG. 26 shows snATAC-seq data highlighting the full length and short center promoter region of the IMPG2 gene.

FIG. 27 shows snATAC-seq data highlighting the promoter and downstream enhancer region of the IMPG1 gene that made up IMPG1-PE1*.

FIG. 28 shows eGFP expression in whole retinal organoids 1-5 (RO1-RO5) transfected with AAV2.7m8 vector containing GFP under the regulation of Round 2 promoters/promoter-enhancer at day 14 (D14) post transduction.

FIG. 29 shows ICC analysis at 14 days post-transduction of human CRX17 ROs transfected with AAV2.7m8 vector containing GFP under the regulation of Round 2 promoters/promoter-enhancer. RCVRN: recoverin.

FIGS. 30A-30C show expression of eGFP and tdTomato in CRX17 ROs (a reporter line in which all PRs express red tdTomato fluorescence) with AAV2.7m8 vector containing GFP under the regulation of Round 2 promoters/promoter-enhancer, by flow cytometry at D14 post-transduction, focusing on PRs. FIG. 21A shows transduction efficiency in PRs, FIG. 21B shows mean fluorescence intensity (MFI) of GFP⁺ PRs, and FIG. 21C shows integrated mean fluorescence intensity (IMFI) of GFP⁺ PRs (iMFI values were calculated as a product of percent transduction efficiency and MFI). Tukey's multiple comparison test was performed and the statistical results are shown in Table 9-11.

FIGS. 31A-31C show expression of eGFP and tdTomato by flow cytometry in CRX17 ROs transfected with AAV2.7m8 vector containing GFP under the regulation of Round 2 promoters/promoter-enhancer, at D14 post-transduction, focusing on non-PRs. FIG. 31A shows the % of total GFP⁺ cells that are GFP⁺ non-PR cells. FIG. 31B shows MFI of GFP⁺ non-PRs. FIG. 31C shows integrated mean fluorescence intensity (iMFI) of GFP⁺ non-PRs (IMFI values were calculated as a product of percent transduction efficiency and MFI). Tukey's multiple comparison test was performed and the statistical results are shown in Table 12-14.

FIG. 32 shows eGFP iMFI ratios of PRs versus eGFP iMFI of non-PRs in CRX17 ROs transfected with AAV2.7m8 vector containing GFP under the regulation of Round 2 promoters/promoter-enhancer. Tukey's multiple comparison test was performed and the statistical results are shown in Table 15.

FIG. 33 shows summary of the results in Round 1 and Round 2 screening (constructs #1-12). #13 and #14 are tested in Round 3.

FIG. 34 shows a summary of the in vivo experiments in cynomolgus macaques and treatment for each eye of each animal.

FIG. 35 shows Optical Coherence Tomography (OCT) imaging of the retinal over time.

FIG. 36 shows Optical Coherence Tomography (OCT) imaging of the retinal over time at lower exposure time.

FIGS. 37A-37B shows activity and specificity of IMPG2-PE1 at 6 weeks post-injection of each animal's eye. FIG. 37A shows the level of co-localization between GFP, PR marker (recoverin), and alpha subunit of a G protein called Go (GO-alpha or GOα) as non-PR marker. FIG. 37B shows the level of co-localization between GFP, PR marker (recoverin), and cellular retinaldehyde-binding protein (CRALBP) as non-PR marker. RCVRN: recoverin.

### DETAILED DESCRIPTION OF THE DISCLOSURE

For the purposes of explicating and understanding the principles of this disclosure, reference is made to embodiments and specific language used to describe the same. The skilled artisan will nevertheless understand that no limitation of the scope of the disclosure is thereby intended, such alteration and further modifications of the disclosure as illustrated herein, being contemplated as would be understood by one skilled in the art to which the disclosure relates.

As used herein, articles “a” and “an” are intended to refer to one or to more than one (i.e., at least one) of the grammatical object of the article. By way of example, “an element” means at least one element and can include more than one element.

“About” is used to provide flexibility to a numerical range endpoint by providing that a given value can be “slightly above” or “slightly below” the endpoint without affecting the therapeutically beneficial result. The term “about” in association with a numerical value means that the numerical value can vary by plus or minus 5% or less of the numerical value. The term “at least” is used to indicate a minimum value in a numerical range.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements, or steps but not the exclusion of any other integer or step or group of integers or steps.

As used herein, “and/or” refers to and encompasses any and all possible combinations of one or more of the associated listed items, as well as the lack of combinations where interpreted in the alternative (“or”).

Recitation of ranges of values herein are merely intended to serve as a succinct method of referring individually to each separate value falling within the range, unless otherwise indicated herein. Furthermore, each separate value is incorporated into the specification as if it were individually recited herein. For example, if a range is stated as 1 to 50, it is intended that values such as 2 to 4, 10 to 30, or 1 to 3, etc., are expressly enumerated in this disclosure. These are only examples of what is specifically intended, and all possible combinations of numerical values between and including the lowest value and the highest value enumerated are to be considered to be expressly stated in this disclosure.

As used herein, the term a “full promoter” or a “full enhancer” refers to the full length of the promoter and the enhancer.

Unless otherwise defined, all technical terms used herein have the same meaning as commonly understood by a person of ordinary skill in the art to which this disclosure belongs.

### I. IMPG1 and IMPG2 Promoter-Enhancer

The disclosure provides a genetically engineered promoter-enhancer construct comprising a promoter and at least one enhancer operatively linked to the promoter, wherein the enhancer specifically or exclusively promotes expression in a photoreceptor cell of a nucleic acid sequence that is operatively linked to the promoter-enhancer construct and capable of being expressed therein.

The terms “express” or “expression” refer to transcription and/or translation of a nucleic acid coding sequence resulting in production of the encoded polypeptide or transcribed nucleic acid. The term “encode” refers to a specific sequence of nucleotides that can be transcribed into RNA or translated into protein.

The term “promoter” refers to a region of DNA upstream of a gene that initiates transcription of that gene, whereby controlling or regulating expression of the gene. As used herein, a promoter can be a photoreceptor-specific promoter. As used herein the term “upstream of a gene” will be understood with regard to the topology of a nucleic acid to indicate the promoter is 5′ to the sequence encoding the gene. In certain embodiments promoters comprising genetically engineered promoter-enhancer constructs provided herein can be a promoter from certain genes, particularly genes expressed in photoreceptor cells and specifically a promoter from an IMPG gene more particularly a human IMPG gene.

The term “enhancer” refers to a sequence that increases the level of transcription of a gene to which it is operatively linked. An enhancer can be upstream or downstream of the gene whose transcription it affects. As used herein the at least one enhancer is photoreceptor-specific and is operatively linked to the promoter and a recombination expression construct comprising the promoter-enhancer is capable of expressing a desired gene in a cell. As used herein the term “downstream of a gene” will be understood with regard to the topology of a nucleic acid to indicate the enhancer is 3′ to the sequence encoding the gene. As used herein the term “upstream of a gene” will be understood with regard to the topology of a nucleic acid to indicate the enhancer is 5′ to the sequence encoding the gene. Also provided are embodiments wherein the enhancer is positioned within the gene, i.e., within an intervening, non-coding (“intron”) sequence. The terms “IMPG1 or IMPG2 downstream enhancer” and “IMPG1 or IMPG2 upstream enhancer” is relative to the topography of the native IMPG1 or IMPG2 genes and not to the promoter that the enhancer is coupled with in the construct. For examples, “IMPG1 downstream enhancer” in “IMPG1 full promoter IMPG1 downstream enhancer” means an enhancer that is downstream of the native IMPG1 gene and does not mean an enhancer sequence that is downstream of the promoter sequence in the construct.

The term “genetically engineered” as used herein refers to nucleic acids or cells that have been manipulated using biotechnology to provide functional capacities or change the genetic makeup of the cells, including the transfer of genes within and across species boundaries to produce improved or non-naturally occurring cells. The term when used with reference to a nucleic acid or a protein generally denotes that the composition or primary sequence of said nucleic acid or protein has been altered from the naturally occurring sequence using experimental manipulations well known to those skilled in the art. The term “genetically engineered” will be understood by the skilled artisan to indicate that the subject matter described thereby has been structurally altered from naturally occurring nucleic acids, and in particularly advantageous embodiments thereof to have altered or enhanced functional properties (for example, capable of providing increased level of expression and/or specificity). A cell such as a human cell that contains an exogenous, recombinant, synthetic, and/or otherwise modified polynucleotide is considered to be a genetically engineered cell and, thus, non-naturally occurring relative to any naturally occurring counterpart. In some cases, genetically engineered cells contain one or more recombinant nucleic acids. In other cases, genetically engineered cells contain one or more synthetic or genetically engineered nucleic acids (e.g., a nucleic acid containing at least one artificially created insertion, deletion, inversion, or substitution relative to the sequence found in its naturally occurring counterpart). Procedures for producing genetically engineered cells are generally known in the art, for example, as described in Sambrook et al, Molecular Cloning, A Laboratory Manual (Fourth Edition), Cold Spring Harbor Press, Cold Spring Harbor, N.Y. (2012) and Doudna et al., CRISPR-Cas, A Laboratory Manual, Cold Spring Harbor Press, Cold Spring Harbor, N.Y. (2016).

As used herein, the term “operatively linked” will be understood by the skilled artisan to mean components that are in a relationship permitting them to function in their intended or conventional manner. For example, a control sequence “operatively linked” to a coding sequence is ligated thereto in such a way that expression of the coding sequence is achieved under conditions compatible with the control sequences. Specifically, for example, genetically engineered promoter-enhancer construct as provided herein is considered to be “operatively linked” to a nucleic acid such as a gene when it is in a correct functional location and orientation in relation to the nucleic acid it regulates to control transcriptional initiation and/or expression of that nucleic acid and the gene encoded thereby.

The term “construct” or “recombinant expression construct” refers to an artificially-designed segment of DNA that can be used to incorporate genetic material into a target cell. Recombinant expression constructs provided herein can comprise genetically engineered promoter-enhancer constructs operably linked to a gene or a gene expression-modulating sequence to be expressed in a recombinant cell. Recombinant expression constructs can further comprise a vector, which the skilled artisan will understand to mean a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.” The term vector as used herein describes any suitable means of directing the expression of nucleic acids in a cell. Examples include, but are not limited to virus, adeno-associated virus, nanoparticles, etc.

The term “sequence identity” refers to the number of identical or similar nucleotide bases on a comparison between a test and reference oligonucleotide or nucleotide sequence. Sequence identity can be determined by sequence alignment of a first nucleic acid sequence to identify regions of similarity or identity to second nucleic acid sequence. As described herein, sequence identity is generally determined by alignment to identify identical residues. Matches, mismatches, and gaps can be identified between compared sequences by techniques known in the art. Alternatively, sequence identity can be determined without taking into account gaps as the number of identical positions/length of the total aligned sequence×100. In one embodiment, the term “at least 90% sequence identity to” refers to percent identities from 90 to 100%, relative to the reference nucleotide sequence. Identity at a level of 90% or more is indicative of the fact that, assuming for exemplary purposes a test and reference polynucleotide sequence length of 100 nucleotides are compared, no more than 10% (i.e., 10 out of 100) of the nucleotides in the test oligonucleotide differ from those of the reference oligonucleotide. Differences are defined as nucleic acid substitutions, insertions, or deletions.

Specifically provided herein are promoter-enhancer constructs for altering transcription of a gene that is operably linked thereto, wherein the constructs comprise genetically engineered promoter-enhancers specific for expression in photoreceptor cells. In some embodiments, the promoter is a modified gene promoter from a gene expressed in a photoreceptor cell, advantageously modified by genetic engineering from an interphotoreceptor matrix proteoglycan (IMPG) gene promoter. The IMPG gene promoter can be an IMPG1 or IMPG2 promoter and in specific embodiments can be the IMPG2 gene promoter. In certain embodiments, some portion of these promoter and/or the enhancer sequences can be removed without abolishing the activity of the promoter-enhancer to regulate gene expression. Because a large base pair size can limit the efficiency of expression of a construct, a shortened and compact promoter-enhancer can in some instances provide more options for larger gene sequences to be packed in the construct. However, as the Examples have shown, the sequence of the full-length promoter and the full-length enhancer cannot be randomly truncated to produce a shorter version that still retains the regulatory activity.

In certain embodiments, the construct includes a promoter and at least one enhancer. In certain embodiments, the enhancer is positioned as a result of genetic engineering upstream, downstream, or near the promoter. In certain embodiments, the promoter is at least 90% identical to SEQ ID NO: 2, SEQ ID NO: 4, or SEQ ID NO: 16. In certain embodiments, the promoter is at least 90% identical to SEQ ID NO: 26. In certain embodiments, the enhancer is at least 90% identical to SEQ ID NO: 1, SEQ ID NO: 6, or SEQ ID NO: 17. In certain embodiments, the enhancer is at least 90% identical to SEQ ID NO: 1, SEQ ID NO: 6, or SEQ ID NO: 25. In certain embodiments provided herein, the genetically engineered promoter-enhancer construct is at least 90% identical to SEQ ID NO: 3, SEQ ID NO: 7, SEQ ID NO: 8, SEQ ID NO: 9, SEQ ID NO: 18, SEQ ID NO: 19, or SEQ ID NO: 20. In certain embodiments provided herein, the genetically engineered promoter-enhancer construct is at least 90% identical to SEQ ID NO: 27.

Compared to GRK1 promoter, which is a conventional promoter used to express a gene specifically in photoreceptor cell, the disclosed IMPG1 and IMPG2 promoter-enhancer constructs are comparable in term of the level of expression and specificity for expression in photoreceptor cells.

The nucleic acid sequence that is operatively linked to the disclosed promoter-enhancer construct can be translated into a polypeptide or transcribed into an RNA in the photoreceptor.

### II. Methods of Expressing a Gene or Modulating a Gene Expression

This disclosure provides a recombinant expression construct comprising the disclosed genetically engineered promoter-enhancer construct. In certain embodiments, the nucleic acid sequence that is operatively linked to the disclosed promoter-enhancer construct encodes a gene or a gene expression modulating sequence that can be expressed in the photoreceptor cell.

Also provided herein is a method of expressing the gene or the gene expression modulating sequence in the photoreceptor cell comprising introducing into the cell the disclosed recombinant expression construct comprising the gene or the gene expression modulating sequence.

The disclosure also provides a method of modulating expression of a target gene in the photoreceptor cell comprising introducing into the cell the disclosed recombinant expression construct.

As used herein the term “target gene” is a specific gene in which its expression is increased or decreased by expression of the encoded gene or the gene modulating sequence. The term “gene modulation” or “gene expression modulation” is intended to encompass increasing or decreasing expression of a gene in a photoreceptor cell. For example, in some embodiments, the encoded gene and the target gene are the same, so that expression of the target gene can be increased by introducing a recombinant expression construct provided herein into a photoreceptor cell and expressing the gene therein. In other examples, expression of the gene can be decreased by introducing a recombinant expression construct provided herein into a photoreceptor cell that encodes a gene modulating sequence such as an siRNA specific for the target gene, the expression of which is intended to be decreased. Alternative embodiments of such gene expression modulating recombinant expression constructs encode components of gene editing systems, for example, Cas9 and/or guide RNA specific for the target gene, the expression of which is intended to be decreased or increased. In these embodiments, expression of the encoded gene editing component results in editing of the target gene so that a mutation or abnormality of the target gene is corrected. In certain embodiments, the target gene is IMPG1 and IMPG2.

As used herein, the term a “gene modulating sequence” refers to a nucleic acid sequence that influences the expression of a gene. In some embodiments, the gene modulating sequence is an siRNA.

In certain embodiments, the recombinant expression construct is introduced in a photoreceptor cell. In certain embodiments, the cell is an animal cell, particularly a mammalian cell. In specific embodiments, the mammalian cell is a human cell. In alternative embodiments the recombinant expression construct cell can be introduced in to the cell in vitro or in vivo.

In some embodiments, the recombinant expression construct alters gene expression in the photoreceptor cell. In some embodiments, gene expression is increased. In some embodiments, gene expression is decreased. In some embodiments, gene expression is abolished. In some embodiments, expression of the gene within the photoreceptor cell is deleterious and reducing or abolishing expression of the gene is advantageous. In some embodiments, low or absent levels of gene expression within the photoreceptor cell are deleterious and increasing gene expression is advantageous. In some embodiments, gene expression is altered temporarily. In some embodiments, gene expression is altered permanently.

In some embodiments, the recombinant expression construct expresses gene editing machinery. In some embodiments, the gene editing machinery is a CRISPR-Cas gene editing system.

In some embodiments, the recombinant expression construct is used to test a gene therapy or cellular therapy. In some embodiments, the recombinant expression construct is used to detect the presence of photoreceptor cells.

In some embodiments, the cell is a stem cell-derived photoreceptor cell. In some embodiments, the cell is an embryonic stem cell-derived photoreceptor cell. In some embodiments, the cell is an induced pluripotent stem cell (iPSC)-derived photoreceptor cell. In some embodiments, the cell is formulated and/or used for therapy.

### III. Pharmaceutical Compositions and Methods of Treatment

Provided herein are pharmaceutical compositions comprising the disclosed recombinant expression constructs comprising the genetically engineered promoter-enhancer construct, or a delivery vehicle comprising the disclosed recombinant expression construct, and a pharmaceutically acceptable carrier.

“Delivery vehicle” refers to any biological material that can transport the recombinant construct to the retina, wherein the recombinant construct comprises the disclosed promoter-enhancer construct operatively linked to the nucleic acid sequence that exhibit therapeutic effects once it is expressed in the photoreceptor cell. A delivery vehicle can be a viral vector (for examples AAV) or non-viral vector (lipid particle, liposome etc.).

“Pharmaceutical composition” as used herein refers to a composition that includes the disclosed recombinant expression construct comprising the genetically engineered promoter-enhancer construct, or a delivery vehicle comprising the disclosed recombinant expression construct comprising the genetically engineered promoter-enhancer construct, and a pharmaceutically acceptable salt, carrier, a solvent, an adjuvant, and/or a diluent, or any combination thereof.

The term “pharmaceutically acceptable vehicle” or “pharmaceutically acceptable carrier” refers to a diluent, adjuvant, excipient, or carrier with which the disclosed recombinant expression constructs comprising the genetically engineered promoter-enhancer construct, or a delivery vehicle comprising the disclosed recombinant expression construct is administered. In various aspects, the pharmaceutically acceptable carrier comprises one or more diluents, preservatives, solubilizers, emulsifiers, adjuvants, excipients, or other carriers.

In certain aspects, the pharmaceutically acceptable carrier comprises one or more excipients or vehicles. Such excipients include liquids such as water, saline, glycerol, polyethyleneglycol, hyaluronic acid, ethanol, balanced salt solution, phosphate buffered saline, and the like.

“Pharmaceutically acceptable carriers” for therapeutic use are well known in the pharmaceutical art, and are described, for example, in Remington's Pharmaceutical Sciences, 18th Edition (Easton, Pennsylvania: Mack Publishing Company, 1990). For example, sterile saline and phosphate-buffered saline at physiological pH can be used. Preservatives, stabilizers, dyes, and even flavoring agents can be provided in the pharmaceutical composition. For example, sodium benzoate, sorbic acid and esters of p-hydroxybenzoic acid can be added as preservatives. Id. at 1449. In addition, antioxidants and suspending agents can be used.

Suitable excipients for non-liquid formulations are also known to those of skill in the art. A thorough discussion of pharmaceutically acceptable excipients and salts is available in Remington's Pharmaceutical Sciences, 18th Edition (Easton, Pennsylvania: Mack Publishing Company, 1990).

Additionally, auxiliary substances, such as wetting or emulsifying agents, biological buffering substances, surfactants, and the like, can be present in such vehicles. A biological buffer can be any solution which is pharmacologically acceptable and which provides the formulation with the desired pH, i.e., a pH in the physiologically acceptable range. Examples of buffer solutions include saline, phosphate buffered saline, Tris buffered saline, Hank's buffered saline, balanced salt solution, and the like.

In general, the compositions of the disclosure will be administered in a therapeutically effective amount by any of the accepted modes of administration. Suitable dosage ranges depend upon numerous factors such as the severity of the disease to be treated, the age and relative health of the subject, the potency of the compound used, the route and form of administration, the indication towards which the administration is directed, and the preferences and experience of the medical practitioner involved. One of ordinary skill in the art of treating such diseases will be able, without undue experimentation and in reliance upon personal knowledge and the disclosure of this application, to ascertain a therapeutically effective amount of the compositions of the disclosure for a given disease.

Also provided herein is a method of treating a retinal disease in a subject comprising administering the disclosed pharmaceutical composition to the subject.

“Treating” or “treatment,” as used herein, covers the treatment of a disorder, condition, or a disease described herein, in a subject, preferably a human, and includes:
i. inhibiting a disease or disorder, i.e., arresting its development;
ii. relieving a disease or disorder, i.e., causing regression of the disorder;
iii. slowing progression of the disorder; and/or
iv. inhibiting, relieving, ameliorating, or slowing progression of one or more symptoms of the disease or disorder. For example, the terms “treating,” “treat,” or “treatment” refer to either preventing development or exacerbation of, providing symptomatic relief for, or curing a patient's disorder, condition, or disease.

As used herein, the term “subject” refers to both human and nonhuman animals. The term “nonhuman animals” of the disclosure includes all vertebrates, e.g., mammals and non-mammals, such as nonhuman primates, sheep, dogs, cats, horses, cows, pigs, chickens, amphibians, reptiles, and the like. The subject can be a human patient that is at risk for, or suffering from, one or more retinal diseases or disorders. The human subject can be of any age (e.g., an infant, child, or adult).

The term “animal cell” will be understood to include, but not be restricted to, cells from dogs, cats, mice, rats, horses, pigs, any non-human primates, and humans. In particular, such cells are retinal cells.

The disclosed IMPG1 and IMPG2 promoter-enhancer can increase expression of a gene that can correct retinal dysfunction. In some embodiments, the genetic abnormalities that cause the retinal diseases, can be corrected by gene editing components that are expressed under the regulation of the IMPG1 and IMPG2 promoter-enhancer.

The in vitro and in vivo data as shown herein demonstrate that IMPG1 and IMPG2 promoter-enhancers are at least as suitable for therapeutic use in primary photoreceptor retinal diseases compared to another photoreceptor-specific promoter, the conventional GRK1 promoter, and offers photoreceptor specificity that other non-specific strong promoters do not possess. In addition, the expression of GRK1, which is a protein involved in phototransduction of the photoreceptor, has been shown to be greatly reduced in primary photoreceptor retinal diseases (Sudharsan et al. Mol Ther. 2025 May 21: S1525-0016(25)00390-9, online ahead of printing). The reduction is likely due to downregulation of GRK1 under these conditions. Therefore, using the conventional GRK1 promoter in retinal diseases is not effective in achieving sufficient expression of a gene or gene expression modulating sequence for therapeutic effects. Conversely, IMPG1 and IMPG2 are structural proteins whose expression are not affected in retinal diseases (Sudharsan et al. Mol Ther. 2025 May 21: S1525-0016(25)00390-9, online ahead of printing); and thus, are more effective than GRK1 promoter in retinal diseases resulting in photoreceptor dysfunction and degeneration. The in vivo data also show that expression driven by IMPG2 promoter-enhancer slowly increases and sustains for a longer period than GRK1 driven expression which quickly reaches peak and decays. Thus, the therapeutic effects brought by IMPG1 or IMPG2 promoter-enhancer can potentially be longer lasting than GRK1. Although strong and non-specific promoters such as CAG promoter can drive a higher expression than IMPG1 and IMPG2 promoter-enhancer, the in vitro data show that these non-specific promoters, in addition to being expressed in off-target, non-photoreceptors, can cause a higher rate of cell death, which is presumably due to toxicity caused by the extreme gene expression and/or off-target effects. Thus, IMPG1 and IMPG2 promoter-enhancer can potentially have a higher safety profile when using in the retina compared to commonly used strong promoters that lack specificity to photoreceptor cells.

In some embodiments, the therapy is used to treat a retinal disease. In some embodiments, the retinal disease is a photoreceptor-specific, inherited retinal disease. In some embodiments, the disease is retinitis pigmentosa. In some embodiments, the disease is Leber congenital amaurosis. In some embodiments, the disease is Usher syndrome. In some embodiments, the disease is Stargardt disease. In some embodiments, the disease is age-related or inherited macular degeneration.

### EXAMPLES

Various exemplary embodiments of compositions and methods according to this invention are now described in the following non-limiting Examples. The Examples are offered for illustrative purposes only and are not intended to limit the scope of this invention in any way. Indeed, various modifications of the invention in addition to those shown and described herein will become apparent to those skilled in the art from the foregoing description and the following examples and fall within the scope of the appended claims.

The Examples set forth herein incorporate and rely on certain experimental and preparatory methods and techniques preformed as exemplified herein.

### Example 1

### Promoter Design

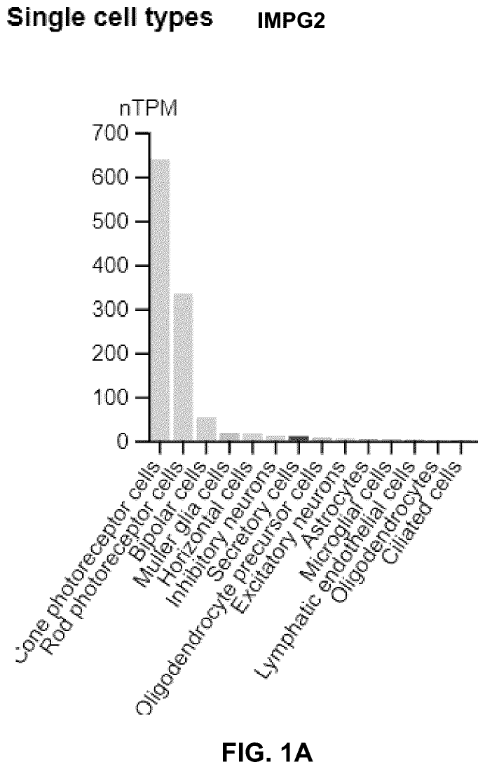

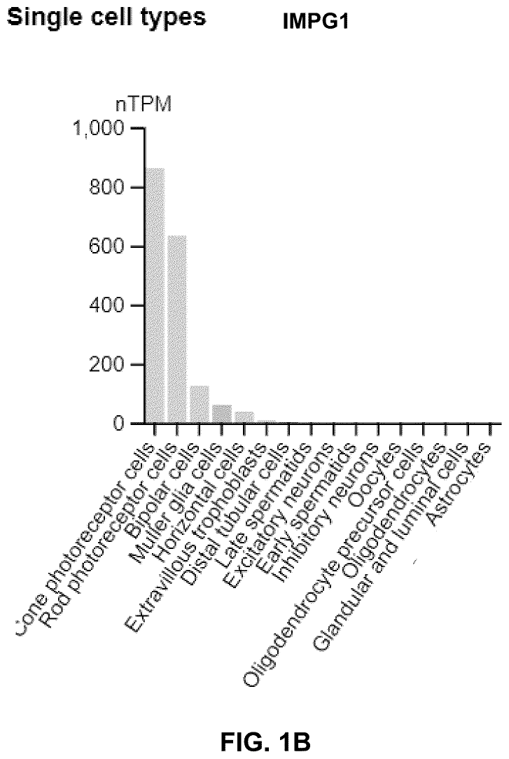

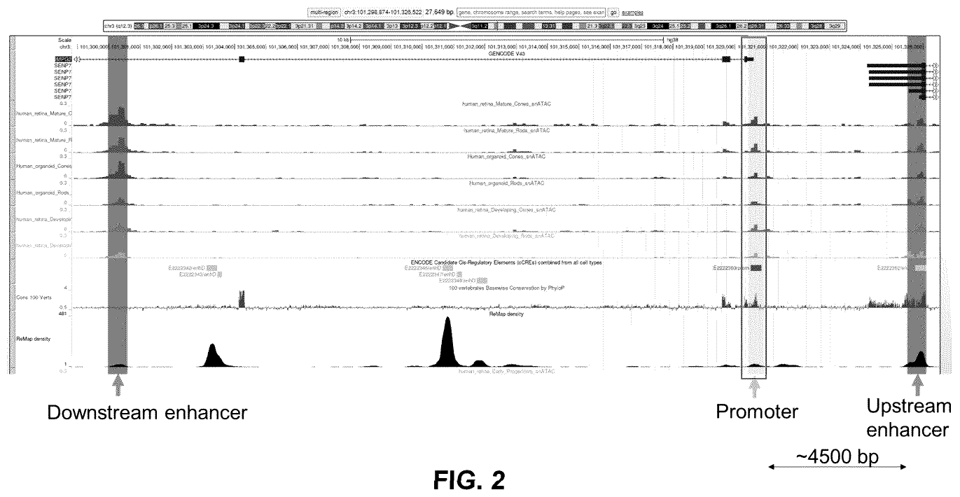

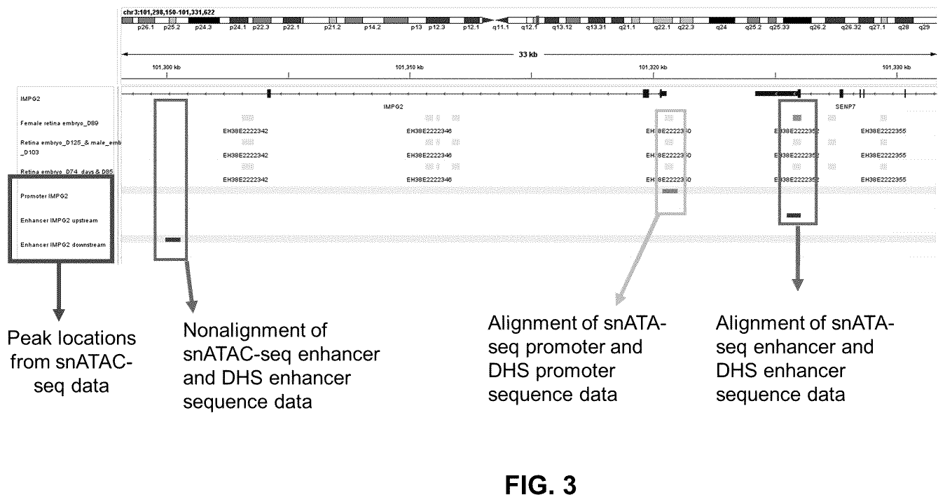

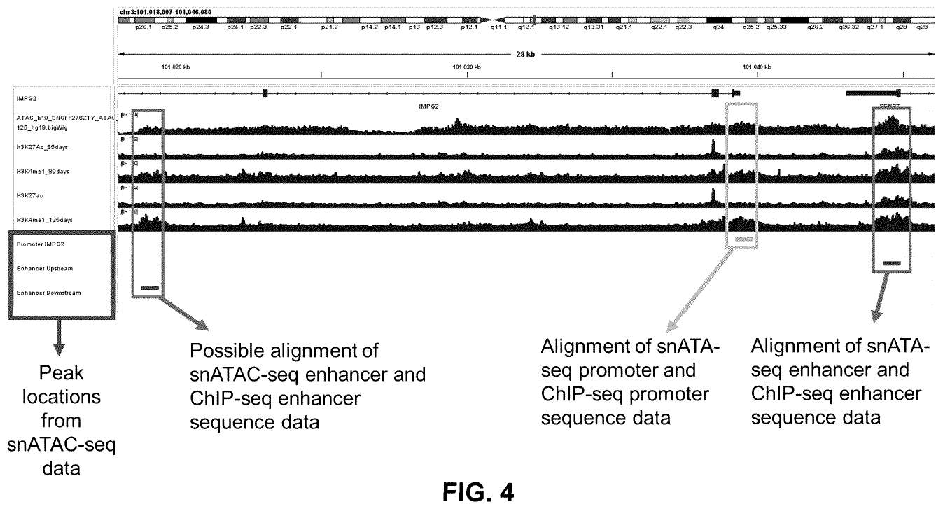

To obtain a photoreceptor-specific promoter, genes having high and specific expression levels in photoreceptors but minimal expression in other cells (including retinal cells such as retinal pigment epithelium (RPE), bipolar cells, and Muller glia) were screened. Two genes were identified using these criteria: IMPG1 and IMPG2; as shown in FIG. 1, genetic analyses in retinal cell types show high and specific expression of IMPG1 and IMPG2 genes in human photoreceptor cells. Informatics analyses were then performed using available datasets from (1) Assay for Transposase-Accessible Chromatin with sequencing (ATAC-seq), (2) Dnase I Hypersensitive Site (DHS) assays, and (3) Chromatin Immunoprecipitation sequencing (ChIP-Seq) (Thomas et al., 2022, “Cell-specific cis-regulatory elements and mechanisms of non-coding genetic disease in human retina and retinal organoids,” Developmental Cell 57:820-835). This comprehensive informatics workflow revealed multiple candidate promoter and enhancer sequences. The peaks identified in FIGS. 2-4 produced by single-nucleus ATAC-seq (snATAC-seq) data analysis indicated candidate cis-regulatory elements (CREs) of the IMPG2 gene in photoreceptor cells of developing and adult human retina and in induced pluripotent stern cell (iPSC)-derived retinal organoids. Utilizing this information, a candidate promoter region was identified directly upstream of the first exon of the IMPG2 gene (highlighted in FIG. 2) (SEQ ID NO: 2). In addition, two candidate enhancer regions were identified (also highlighted in FIG. 2) upstream and downstream of this promoter.

### Example 2

In Vitro Screening of Candidate Promoters Using Bicistronic eGFP/Luciferase Reporters

To assess the relative retina-specific activities of photoreceptor-specific putative promoter sequences identified by the informatics analysis disclosed in Example 1, a two-tier in vitro system was used employing human embryonic kidney (HEK-293) cells and an immortalized human retinoblastoma cell line (Y79), which expresses low levels of numerous photoreceptor proteins. Y79 and HEK cell lines were selected to determine specific and non-specific activity of the promoters, respectively. HEK-293 cells were obtained from ATCC and maintained in DMEM containing 10% fetal bovine serum (FBS). Y79 (HTB-18) cells were obtained from ATCC and maintained in RPMI-1640 medium containing 20% FBS.

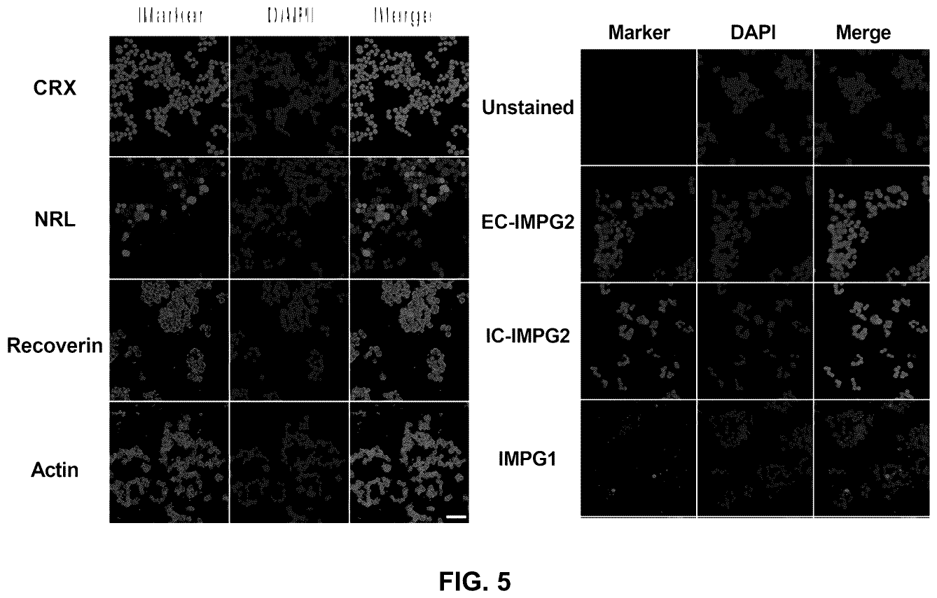

To validate their suitability for use in the screen, Y79 cells were plated on a 96-well plate and stained with the following photoreceptor (PR)-specific marker antibodies: IMPG2 extracellular domain (EC-IMPG2), IMPG2 intracellular domain (IC-IMPG2), IMPG1, Cone-Rod Homeobox (CRX), Neural Retina Leucine Zipper (NRL), and recoverin. Unstained cells were used as a negative control and cells stained for actin were used as a positive control. Immunocytochemistry analyses of Y79 cells showed significant expression of all PR markers tested except IMPG1, whose expression level was low (shown in FIG. 5). These data suggested that Y79 cells could be used as a high-throughput proxy for retinal organoids to perform initial screening of PR-specific promoters.

To carry out these experiments, bicistronic plasmids expressing luciferase-P2A-eGFP reporter constructs under the control of one of four different test promoters were synthesized (Genscript). Ubiquitous promoters such as cytomegalovirus (CMV) and chicken beta-actin (CAG) promoters were used as positive controls because they are known to direct transcription nonspecifically at high levels in many cell lines, and untransfected cells were used as negative controls. In addition, the photoreceptor-specific GRK1 promoter was used as a positive control.

Cells were plated on poly-D lysine-coated 96-well plates. One day after plating, cells were transfected with the respective plasmids as follows. Briefly, plasmids were mixed with TransIT-X2 transfection (Mirus Bio) reagent according to the manufacturer's instructions and added dropwise to the cells. Luciferase assays were performed 2-4 days post transfection using the Dual-Glo luciferase assay kit (Promega) according to the manufacturer's instructions. Luminescence was measured using a Glomax multidetection luminometer (Promega).

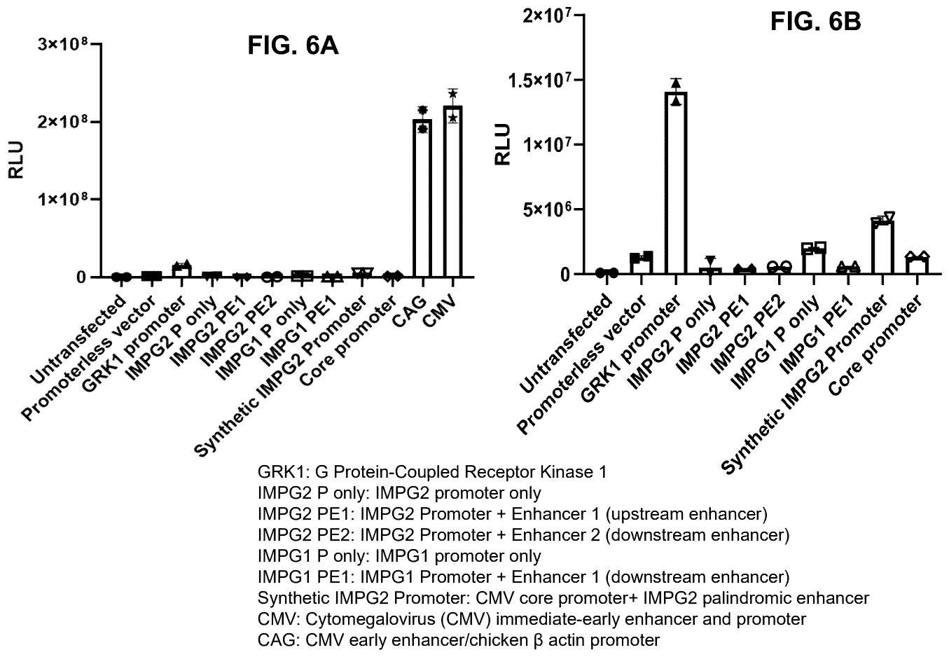

The resultant relative luminescence (RLU) values showed high and non-specific expression of constitutive CAG and CMV promoters in HEK cells, as expected (shown in FIG. 6A). Because of the very high relative levels of CAG and CMV expression, other test plasmids appeared to show little or no expression in HEK cells. However, when the CAG and CMV outlier test groups are removed from the analysis, differences between non-CAG and non-CMV test promoters become evident. As HEK cells are derived from kidney and not retina, luciferase was not expected to be expected from plasmids containing photoreceptor (PR)-specific promoters. GRK1, a presumed photoreceptor (PR)-specific promoter surprisingly exhibited moderately high, non-specific expression in HEK cells. The rest of the test PR promoters, including IMPG2-PE1, did not drive expression in HEK cells appreciably (shown in FIG. 6B).

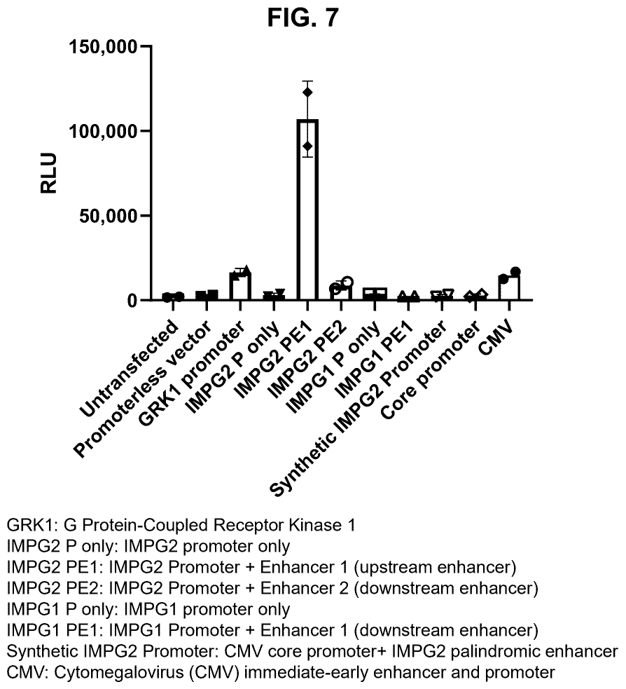

Similarly, removing outlier CAG promoter data unmasked the results from screening in Y79 cells. The IMPG2 PEI test promoter (which incorporated the IMPG2 promoter and IMPG2 upstream enhancer identified in Example 1, shown in FIG. 2) exhibited the highest activity in Y79 cells (shown in FIG. 7). All other test PR promoter candidates showed low or baseline activity.

Overall, the genetically engineered IMPG2-PE1 promoter (SEQ ID NO: 3), comprising an enhancer identified herein by SEQ ID NO: 1, showed the highest Y79-specific activity amongst all the candidate Promoter-Enhancer (PE) sequence combinations tested; therefore, it was selected as the lead candidate to be tested in retinal organoids using adeno-associated virus (AAV)-mediated viral transduction.

### Example 3

### In Vitro Assessment of Candidate Promoters in Retinal Organoids (ROs)

In order to qualitatively test relative PR-specific activity of the genetically engineered IMPG2 PEI promoter in authentic human PRs, a human pluripotent stem cell (hPSC)-derived retinal organoid (RO) model system was employed. To transduce these ROs, adeno-associated virus 2.7m8 (AAV2.7m8) constructs were created, inter alia because AAVs are the most efficient and commonly used delivery vectors for gene- and genome-based therapeutics. The methods of U.S. Patent Publication No. US20110081719A1, and U.S. Patent Publication No. US20160251618A1, were used in these experiments and are incorporated herein by reference.

AAV2.7m8 constructs containing reporter gene eGFP under the regulation of one of four test promoters (CAG, CMV, IMPG2 PE1, and synthetic) were commercially synthesized by Vector Builder. The synthetic IMPG2 promoter included a CMV core promoter and an enhancer sequence, wherein the enhancer sequence was based on a previously reported palindromic sequence (Govorkova et al., 2022, ACS Synthetic Biology 11 (3): 1096-1105). Stem cells from the CRX-17 tdTomato PR reporter line (in which only PRs express tdTomato) were cultured and differentiated to PR-containing ROs using a previously published protocol (Phillips et al., 2018, Stem Cells 36(3): 313-324). Briefly, pluripotent stem cell colonies were detached from cell culture plates to form embryoid bodies (EBs). EBs were transitioned from mTeSR plus to a neural induction medium (NIM, composed of a 1:1 mixture of DMEM and F12 media containing 1% N2 supplement, 1×MEM nonessential amino acids (MEM NEAA), 1×GlutaMAX (Thermo Fisher) and 2 ug/ml heparin (Sigma) over the course of 4 days. On day 6 (D6), 0.75 nM bovine morphogenic protein 4 (BMP4; R&D Systems) was added to fresh NIM and one day later, EBs were plated on Matrigel at a density of 200 EBs per well of a 6-well plate. Half the media was replaced with fresh NIM on D9, D12, and D15, and on D16 the media was changed to Retinal Differentiation Medium (RDM; DMEM: F12 3:1, 2% B27 supplement, MEM NEAA, 1×antibiotic, antimycotic (Thermo Fisher) and 1× GlutaMAX). On D25-D30, optic vesicle-like colonies became visually apparent by brightfield microscopy and were dissected with an MSP ophthalmic surgical knife (Surgical Specialties Corporation). The resulting free-floating retinal organoids were maintained in poly-HEMA-coated flasks (polyHEMA, Sigma) with twice-weekly feeding of 3D-RDM (DMEM: F12 3:1, 2% B27 supplement, 1×MEM NEAA, 1× antibiotic, anti-mycotic, and 1× GlutaMAX with 5% FBS, 100 μM taurine, 1:1000 chemically defined lipid supplement (11905031, Thermo Fisher). Between day 100 and day 115 of differentiation, ROs were treated overnight with ViraDuctin (Cell Biolabs) according to the manufacturer's instructions, and the following day were transduced with the AAV vectors. Expression of eGFP was assessed in live ROs 13 days after transduction using a Nikon Ts2-FL microscope equipped with a DS-Fi3 camera.

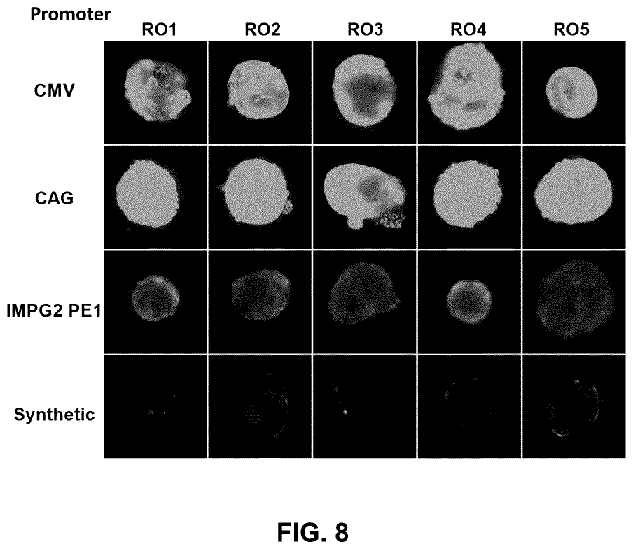

Both nonspecific promoters (CAG and CMV promoters) showed high, indiscriminate GFP expression throughout all ROs. However, the IMPG2 PEI promoter showed expression of eGFP only in the outer mantle of ROs, where PRs are located, and the synthetic PR promoter did not demonstrate appreciable eGFP fluorescence anywhere within the ROs (shown in FIG. 8). This eGFP expression pattern provided qualitative evidence of human PR-specific promoter activity exhibited by IMPG2 PE1.

Next, flow cytometry was performed to quantitively assess the PR (i.e., on-target) promoter activity of IMPG2 PEI in hPSC-derived ROs relative to ubiquitous promoters.

Transduced CRX-17 tdTomato ROs from the experiments just described in this Example were dissociated at day 14 post-transduction into single cell suspensions using 10× TrypLE (ThermoFisher). Flow cytometry of live cells was then performed using ThermoFisher Attune, which included compensation and fluorescence minus one (FMO) controls to determine the gates. Data were analyzed using FlowJo software version 10.9. The PR transduction efficiency percentages and median fluorescence intensities (MFI) in PRs was determined by virtue of their dual positivity for tdTomato (red) and eGFP (green) fluorescence. Integrated MFI (iMFI) values were calculated as a product of percent transduction efficiency and MFI.

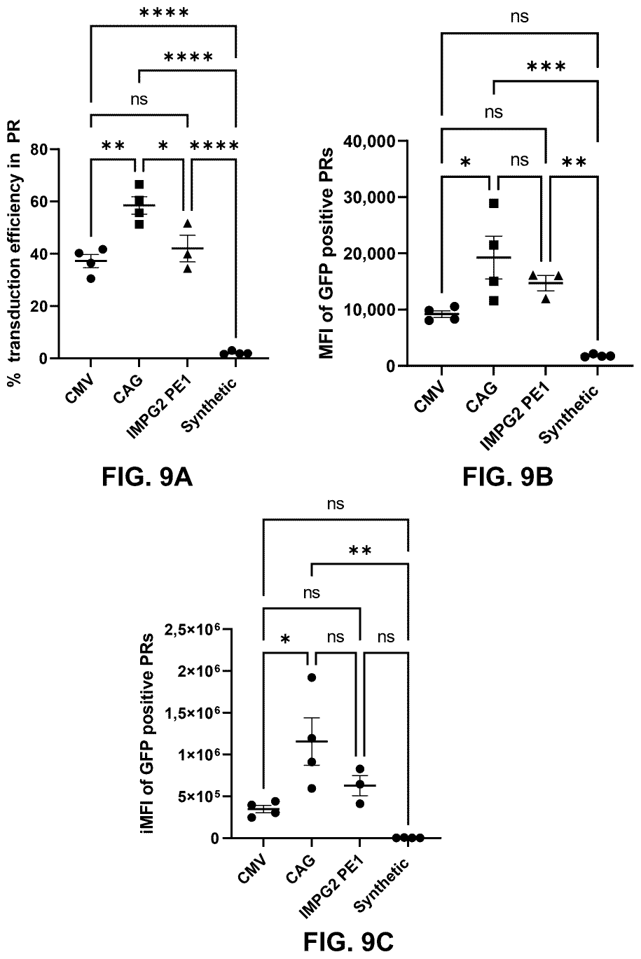

The AAV2.7m8 vector containing the CAG promoter exhibited the highest iMFI values in PRs, indicating that, because CAG is nonspecific and thus mediates expression in any cell type, it unsurprisingly showed expression in PRs at a very high levels. In contrast, however, the IMPG2 PEI promoter showed eGFP reporter gene expression in PRs at a similar level to CAG (i.e., the difference between them was statistically non-significant). Additionally, IMPG2 PEI showed eGFP reporter gene activity at a similar or greater level than the ubiquitous CMV promoter (shown in FIGS. 9B-9C). Thus, the IMPG2 PEI promoter supported gene expression in RO-derived PRs at levels equivalent to highly active, ubiquitously expressing CAG or CMV promoters and was more specific for PRs than those promoters.

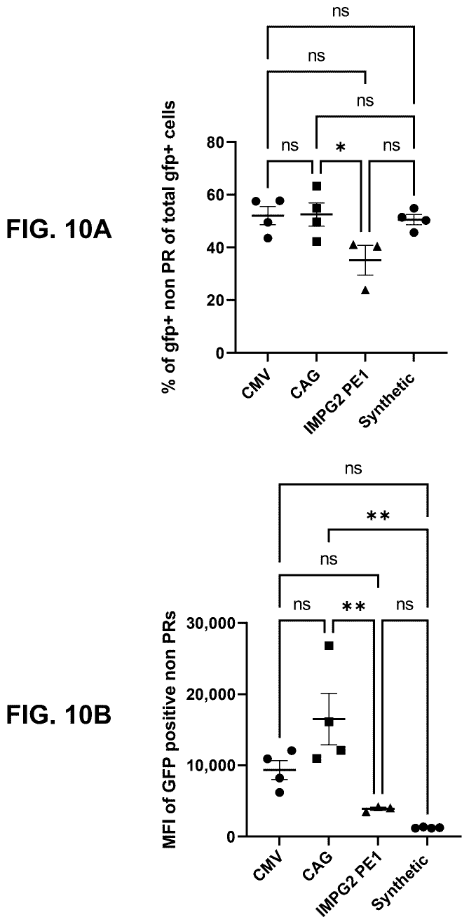

In parallel, flow cytometry was used to analyze cells that were positive for eGFP (green) fluorescence but negative for tdTomato (red) fluorescence (i.e., non-PR cells) to assess off-target activity. As expected, the ubiquitous CAG promoter exhibited the highest transduction percentage and mean fluorescent intensity (MFI) values in non-PRs, confirming its nonspecific gene expression capacity. The IMPG2 PEI promoter showed significantly lower eGFP reporter activity in non-PRs compared to the CAG promoter. In addition, the activity of IMPG2 PEI in non-PRs was lower than that of the ubiquitous CMV promoter, albeit not a significant level (shown in FIGS. 10A-10B). These results showed that the IMPG2 PEI promoter had less activity in non-PRs than ubiquitous promoters, further confirming its specificity for PRs.

Finally, flow cytometry analyses shown in FIG. 9 and FIG. 10 were combined to quantitatively determine the overall PR-specific activity of the IMPG2 PEI promoter in hPSC-ROs relative to ubiquitous promoters. The fold change of eGFP fluorescence iMFI values in PRs versus non-PRs was calculated for each tested promoter by dividing expression level values from the former by expression level values from latter.

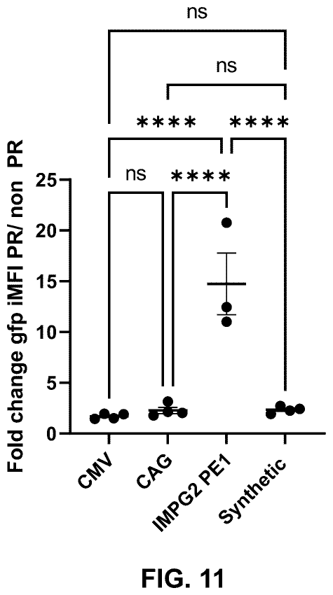

The ratio of eGFP iMFI in PRs to the eGFP iMFI in non-PRs revealed significantly higher PR specificity of the IMPG2 PEI promoter compared to the CAG and CMV ubiquitous promoters, as well as the synthetic promoter negative control (shown in FIG. 11).

Cumulative analysis of all parameters quantitatively assessed by flow cytometry showed superiority (>10-fold) of the IMPG2 PEI promoter compared to commonly used ubiquitous promoters.

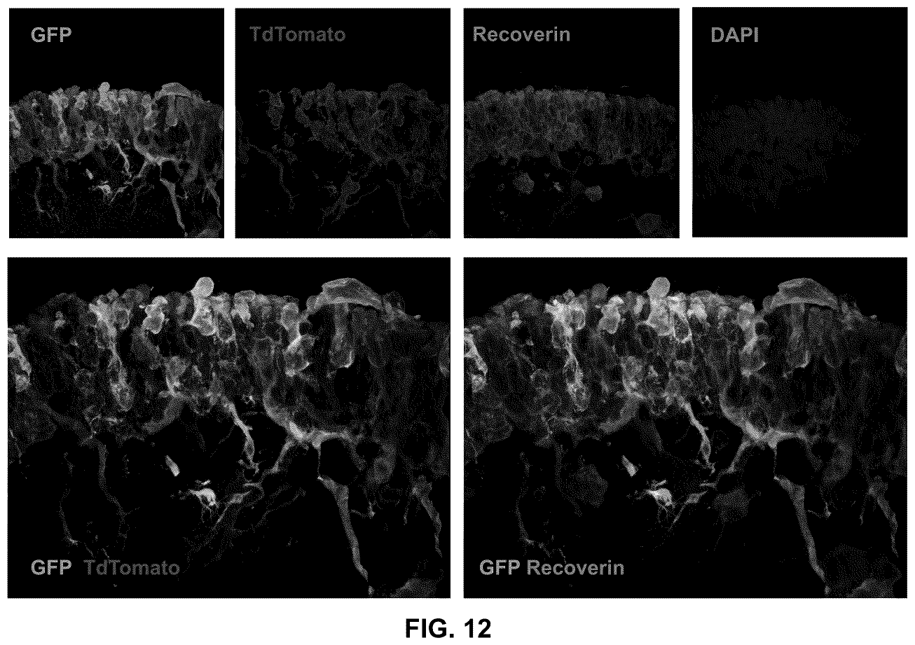

To further corroborate the flow cytometry findings detailed in FIGS. 9-11, immunocytochemistry (ICC) analysis was performed on intact human CRX17 ROs transduced with AAV2.7m8 vectors expressing eGFP under the control of the IMPG2 PEI, CAG, or CMV promoters. A cohort of ROs from the experiments used for the analyses shown in FIG. 9 and FIG. 10 (14 days post-transduction) were fixed, cryopreserved, cryosectioned, and immunostained using primary antibodies directed against GFP (green), tdTomato (red), and recoverin (purple). Thereafter, the immunostained cryosections were imaged using a Nikon A1 laser scanning confocal microscope with NIS Elements AR 5.0 software. IMPG2 PE1-driven GFP expression strongly co-localized with the PR reporter TdTomato and the PR marker recoverin (shown FIG. 12). These data further supported the flow cytometry findings that expression of eGFP driven by the IMPG2 PEI promoter was highly specific to PRs.

### Example 4

### In Vivo Testing of Candidate Promoters in Mouse Retinas

To examine in vivo activity of IMPG2 PE1, mouse retina was transduced with AAV2.7m8 vectors expressing eGFP under the control of either IMPG2 PEI or CAG promoters. AAV2.7m8 vectors harboring either an IMPG2 PE1-eGFP or a CAG-eGFP reporter expression construct were injected into the subretinal space of wildtype mice. Twenty-eight days later, eyes were removed, fixed, sectioned, and processed for brightfield and eGFP fluorescence. Cryosections were blocked and immunostained with primary antibodies directed against GFP and the PR marker recoverin. Immunostained cryosections were imaged using a Nikon A1 laser scanning confocal microscope and NIS Elements AR 5.0 software.

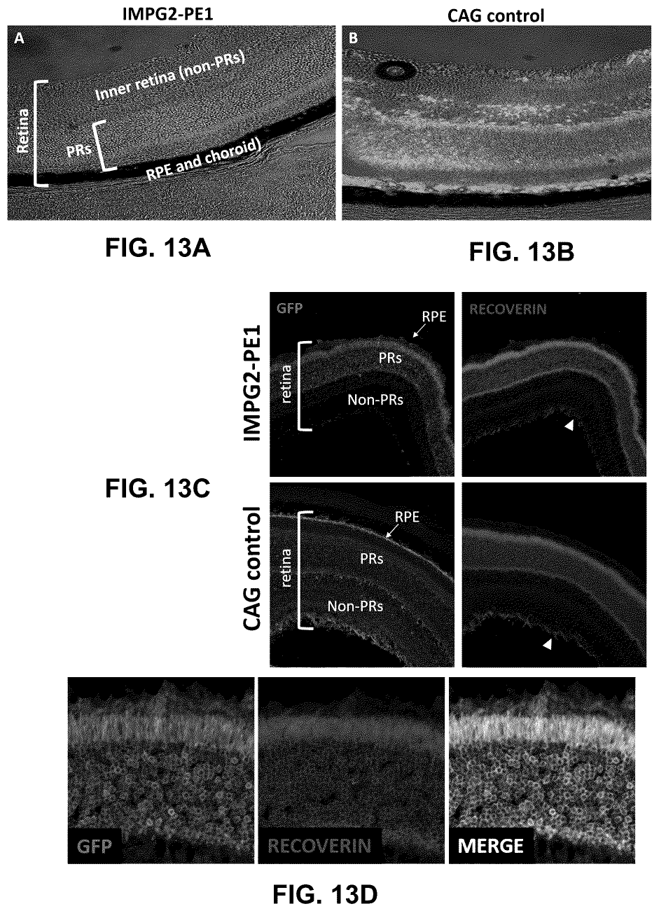

Consistent with the results in cell lines and retinal organoids, the use of the IMPG2 PEI promoter resulted in PR-specific eGFP expression (shown in FIG. 13A), whereas the use of the ubiquitous CAG promoter yielded nonspecific eGFP expression throughout the retina (shown in FIG. 13B). Moreover, the pattern of eGFP expression under control of the IMPG2 PEI promotor coincided strictly with the immunostaining pattern of the PR marker recoverin (shown FIG. 13C, top two panels), whereas the pattern of eGFP expression under the control of the CAG promotor included all retinal cells (shown FIG. 13C, bottom two panels). FIG. 13D is a higher magnification image showing the complete overlap of IMPG2 PE1-eGFP expression in RECOVERIN+ PRs. Of note, recoverin was also expressed in some bipolar cells (shown by arrowheads in FIG. 13C), but no eGFP expression was seen in these non-PRs using IMPG2 PE1, in contrast to the results where eGFP expression was mediated by the CAG promoter. This further demonstrated the PR specificity of the IMPG2 PEI promoter.

In sum, the in vivo mouse retina data confirmed the PR-specific promoter activity of IMPG2 PE1.

### Example 5

In Vitro Assessment of Candidate Promoters in iPSC-Derived Retinal Organoids (ROs)

These findings were further validated in ROs differentiated from a human induced pluripotent stem cell (iPSC) 10.13 line. Stem cells from the 10.13 cell line were cultured and differentiated to ROs. Prior to transduction, ROs were treated overnight with ViraDuctin (Cell Biolabs) according to the manufacturer's instructions. The following day, ROs were infected with AAV2.7m8 vector containing IMPG2 PEI promoter sequences driving eGFP as a reporter gene. At 21-28 days post-treatment (DPT) transduced ROs were fixed in 4% paraformaldehyde (Electron Microscopy Sciences) at room temperature (RT) with gentle agitation for 40 mins, washed with 1×PBS, and cryopreserved in 15% sucrose in PBS for 40 min, followed by equilibration in 30% sucrose for 40 min. Retinal organoids were flash frozen immediately post-equilibration and then cryosectioned at a thickness of 15 μm. Cryosections were blocked in blocking solution (10% normal donkey serum, 5% BSA, and 0.5% Triton X-100 in 1×PBS) for 1 h at room temperature and incubated at 4° C. overnight with primary antibodies (anti-GFP and anti-recoverin) prepared in blocking solution. After incubation, cryosections were washed three times in phosphate buffered saline (PBS), then incubated for 30 min in the dark at room temperature with appropriate fluorophore-conjugated secondary antibodies prepared in blocking solution. Thereafter, the immunostained cryosections were washed three times in PBS, mounted in Prolong Gold Antifade with DAPI (Thermo Fisher Scientific), and imaged using a Nikon A1 laser scanning confocal microscope with NIS Elements AR 5.0 software.

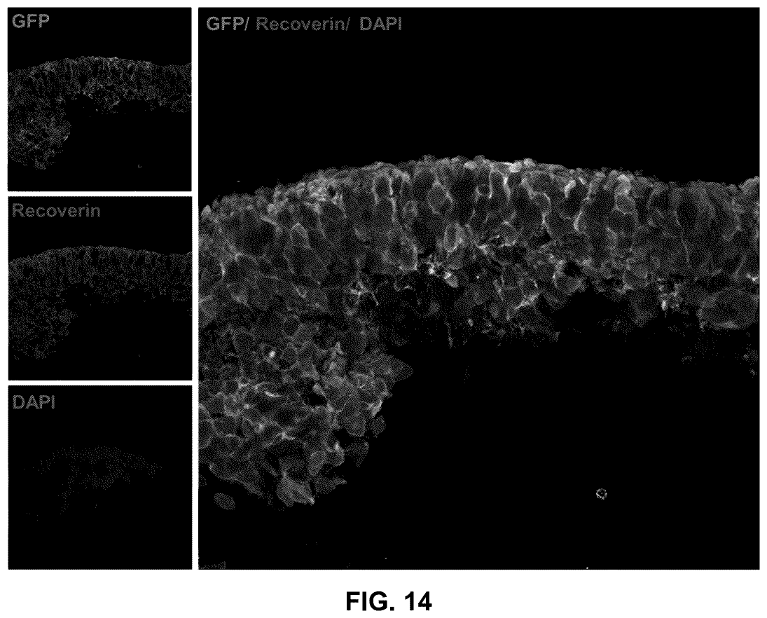

ICC data showed IMPG2 PEI to be active in the photoreceptor cells of all ROs (shown in FIG. 14). Of note, CRX17 ROs from Example 3 were derived from embryonic stem cells (ESC) line. Thus, these data from that example and this example demonstrate the activity and specificity of IMPG2 PEI in both ESC and iPSCs.

### Example 6

In Vitro Assessment of Candidate Promoters in Retinal Organoids (ROs) from Another Species

Cross-species (i.e., in a preclinical animal model) activity of IMPG2-PE1 was tested in ROs derived from pig iPSCs.

Pig stem cells were cultured and differentiated to retina cells. Prior to transduction, ROs were treated overnight with ViraDuctin (Cell Biolabs) according to the manufacturer's instructions. The following day, ROs were infected with AAV2.7m8 vector containing IMPG2 PEI promoter sequences driving eGFP as a reporter gene. At 21 DPT transduced ROs were fixed in 4% paraformaldehyde (Electron Microscopy Sciences) at room temperature (RT) with gentle agitation for 40 mins, washed with 1×PBS, and cryopreserved in 15% sucrose in PBS for 40 min, followed by equilibration in 30% sucrose for 40 min. Retinal organoids were flash frozen immediately post-equilibration and then cryosectioned at a thickness of 15 μm. Cryosections were blocked in blocking solution (10% normal donkey serum, 5% BSA, and 0.5% Triton X-100 in 1×PBS) for 1 h at room temperature and incubated at 4° C. overnight with primary antibodies (anti-GFP and anti-recoverin) prepared in blocking solution. After incubation, cryosections were washed three times in phosphate buffered saline (PBS), then incubated for 30 min in the dark at room temperature with appropriate fluorophore-conjugated secondary antibodies prepared in blocking solution. Thereafter, the immunostained cryosections were washed three times in PBS, mounted in Prolong Gold Antifade with DAPI (Thermo Fisher Scientific), and imaged using a Nikon A1 laser scanning confocal microscope with NIS Elements AR 5.0 software.

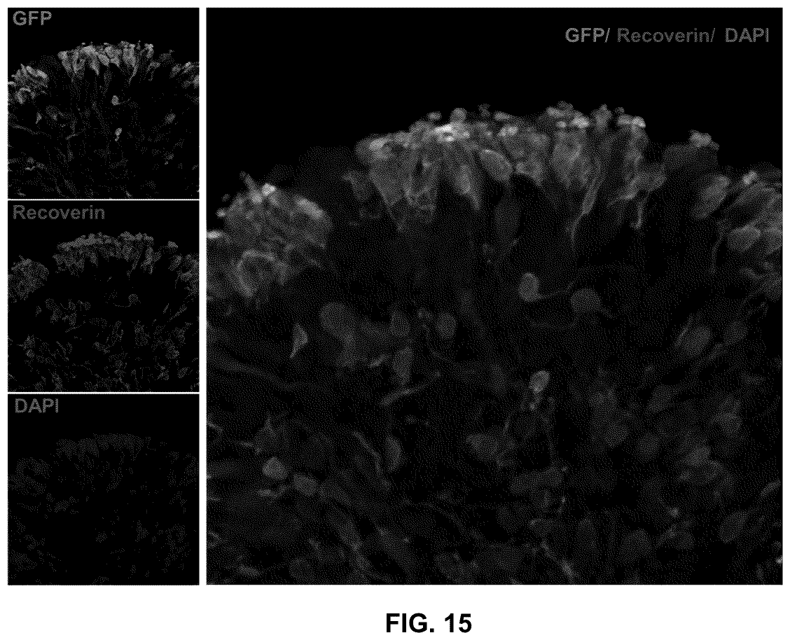

Similar to its expression in human ROs (FIG. 14), pig ROs showed high and photoreceptor-specific activity of IMPG2-PE1 (shown in FIG. 15). Altogether, the activity of IMPG2-PE1 in Y79 retinoblastoma cells, HEK cells, and human and pig pluripotent stem cell-derived ROs showed it to be highly active and significantly more photoreceptor-specific than other available promoters.

### Example 7

### Round 1: Designing Shortened Promoter-Enhancer Constructs

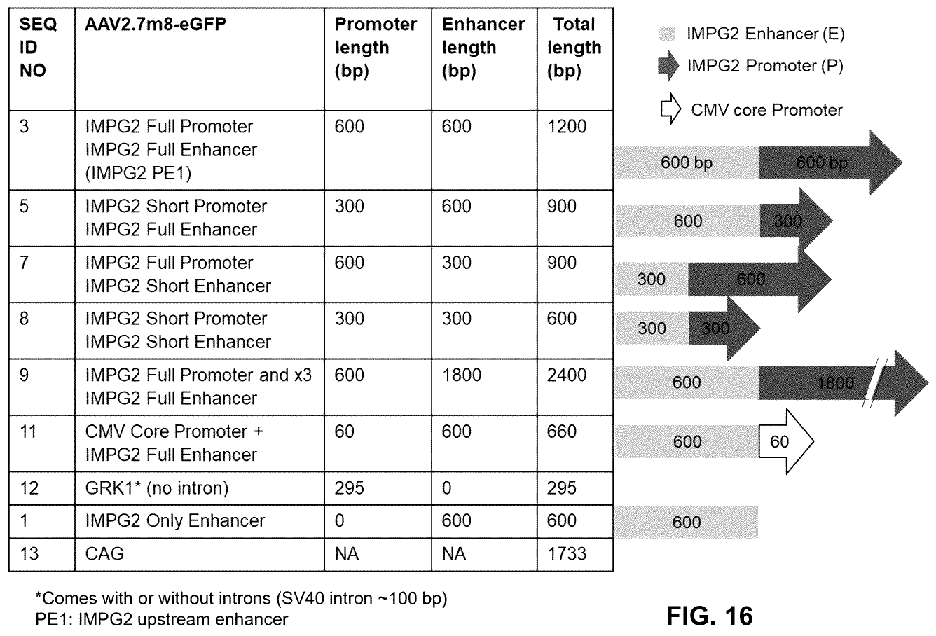

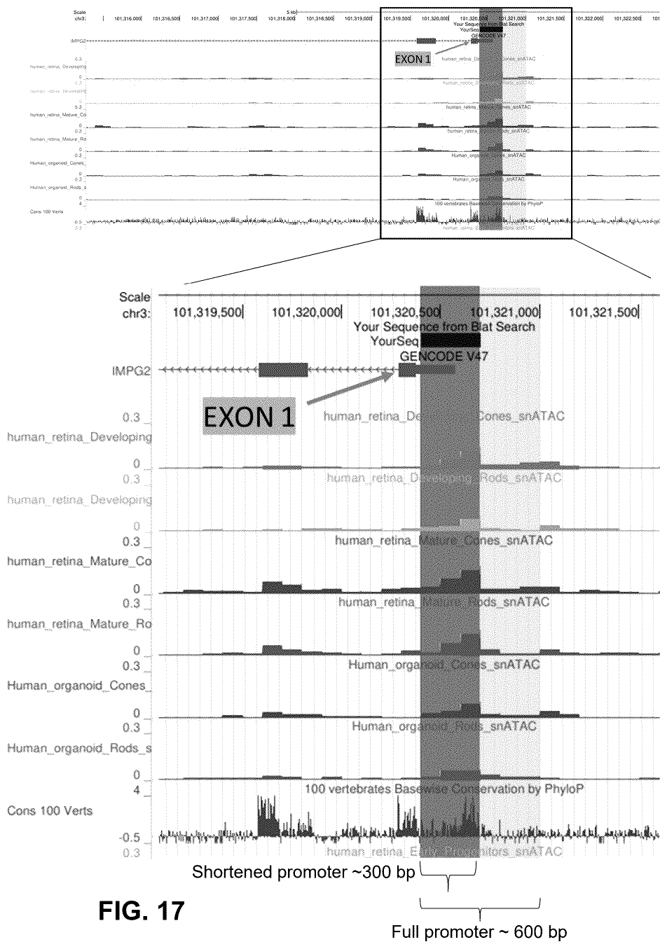

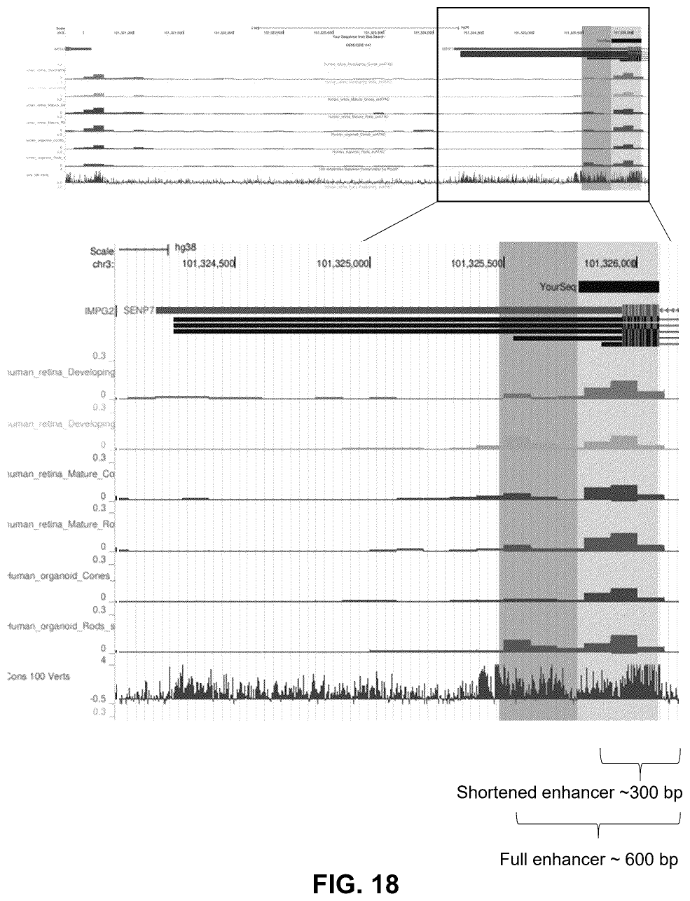

A short promoter-enhancer allows more space for packaging of larger transgenes into a construct. Thus, the IMPG2-PE1 promoter-enhancer construct which comprised about 600 base pairs (bp) promoter and 600 bp enhancer, was designed to reduce its total length by shortening the promoter region, the enhancer region, or both (FIG. 16 to FIG. 18). Specifically, the second half of the full length IMPG2 promoter, which was shown to have fewer transcription binding sites than the first half, was removed to produce a shortened IMPG2 promoter of about 300 bp (FIG. 17). Similarly, the second half of the full length IMPG2 upstream enhancer was removed to produce a shortened enhancer of about 300 bp (FIG. 18). Nine promoters were examined for level of expression of eGFP and PR-specificity in an hPSC-derived RO model system, in similar experiments to Example 3. The AAV2.7m8 constructs containing reporter gene eGFP under the regulation of each of the test promoters were utilized. Five designs of different lengths of IMPG2 promoter and its enhancer were tested: full length IMPG2 promoter and IMPG2 full length enhancer (IMPG2-PE1), short IMPG2 promoter and IMPG2 full length enhancer, full IMPG2 promoter and IMPG2 short enhancer, short IMPG2 promoter and IMPG2 short enhancer, and full length IMPG2 promoter with three units of IMPG2 full length enhancer. GRK1, known to be a standard promoter specific for PR, non-specific CMV promoter-enhancer, and non-specific CAG promoter were used as positive controls. Constructs containing only enhancer without its IMPG2 promoter was also tested to determine if the enhancer only was sufficient to drive expression. A summary of the tested constructs and their respective lengths are shown in FIG. 16.

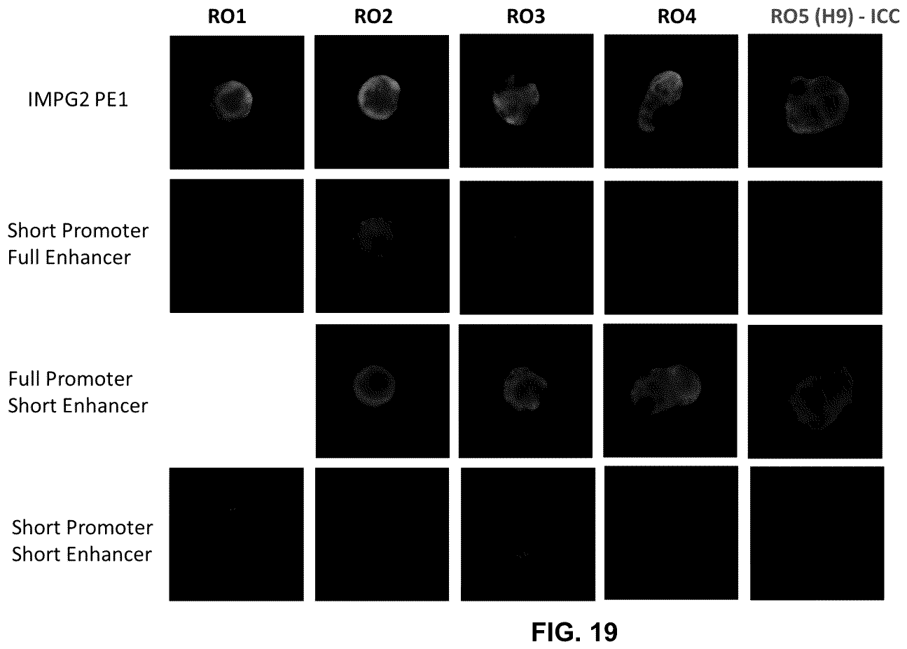

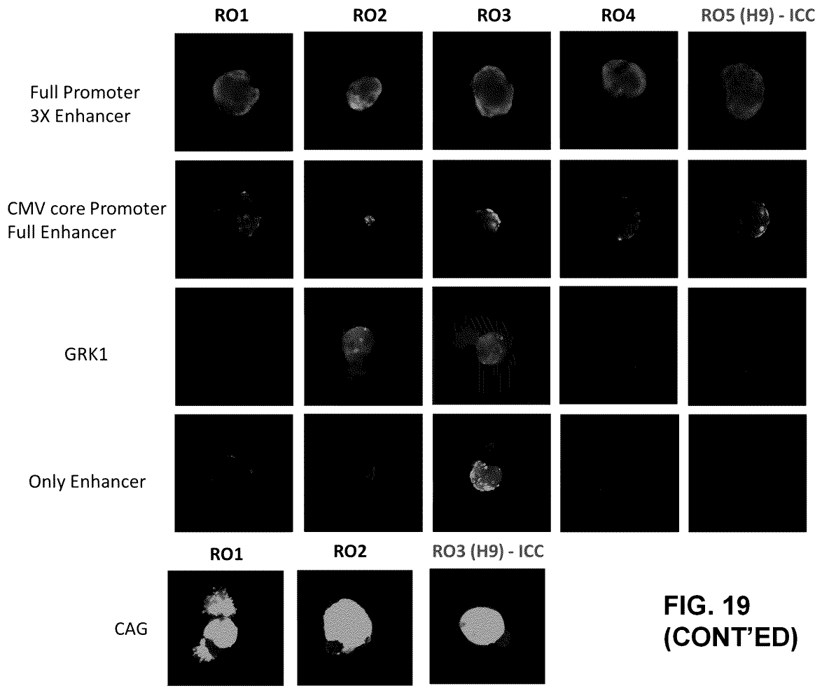

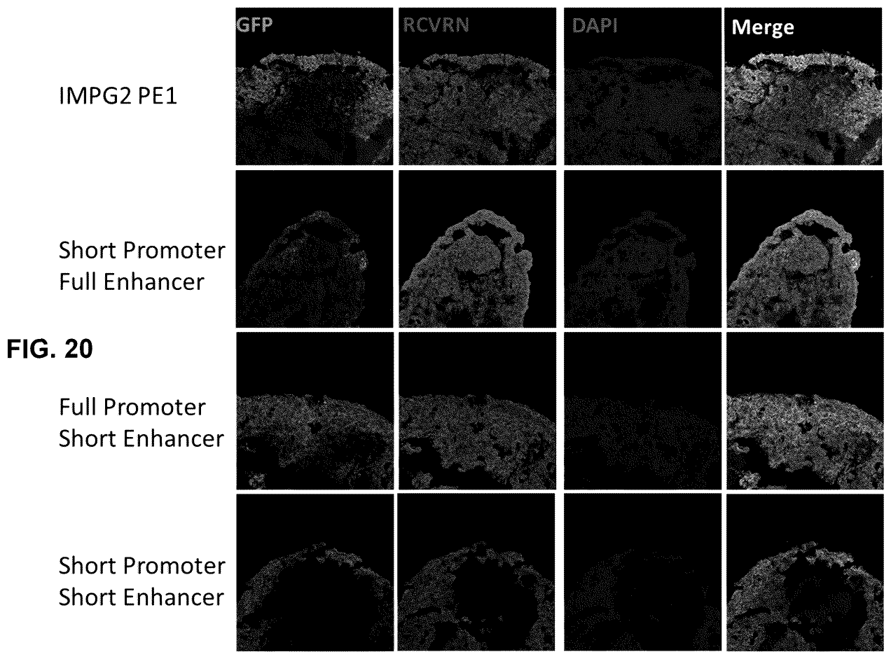

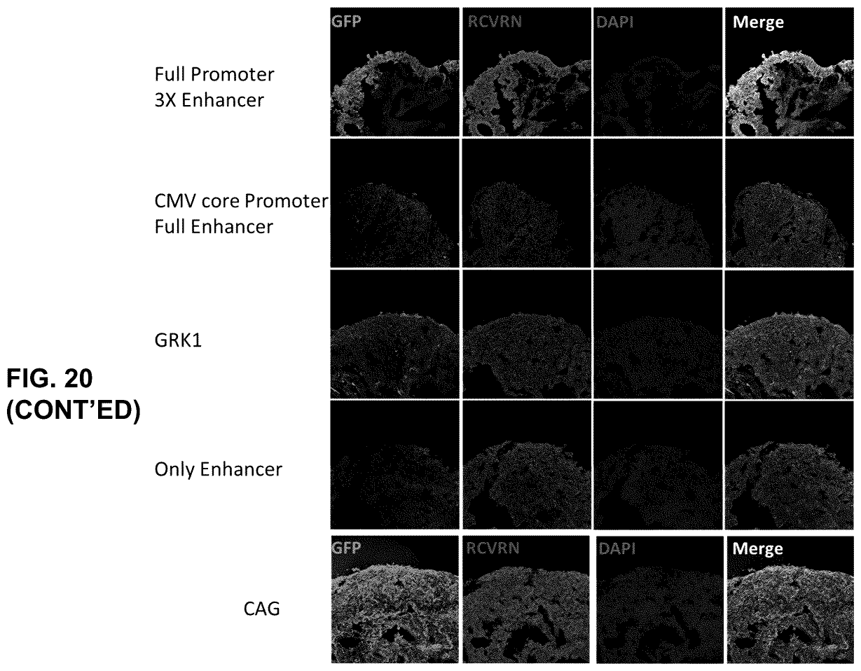

The results showed that shortening only the enhancer (full IMPG2 promoter and IMPG2 short enhancer) did not reduce GFP expression (FIGS. 19-21C) or affect PR specificity (FIGS. 19, 20, 22A-22C, and 23). Adding 3 units of enhancers (full length IMPG2 promoter with three units of IMPG2 full length enhancer) also did not change the expression (FIG. 19-21C) or specificity profile (FIGS. 19, 20, 22A-22C, and 23) compared to the full construct IMPG2-PE1.

In contrast, shortening the promoter reduced the expression level regardless of the length of the enhancer (short IMPG2 promoter and IMPG2 full length enhancer, and short IMPG2 promoter and IMPG2 short enhancer, FIGS. 19-21C).

Surprisingly, although the level of expression was reduced by shortening the promoter, the reduction was less if the enhancer was also shortened (full IMPG2 promoter and IMPG2 short enhancer vs short IMPG2 promoter and IMPG2 short enhancer).

In these experiments, CAG promoter showed the highest expression of GFP but this expression was very non-specific to PR (FIGS. 19-23). However, ROs transfected with CAG showed higher rate of death (FIG. 24) compared to other tested promoter constructs, possibly due to toxicity caused by high expression of GFP. This result indicated that a balanced expression provided by IMPG2-based promoters was more desirable for safety profile.

### Example 8

### Round 2: Designing Shortened Promoter-Enhancer Constructs

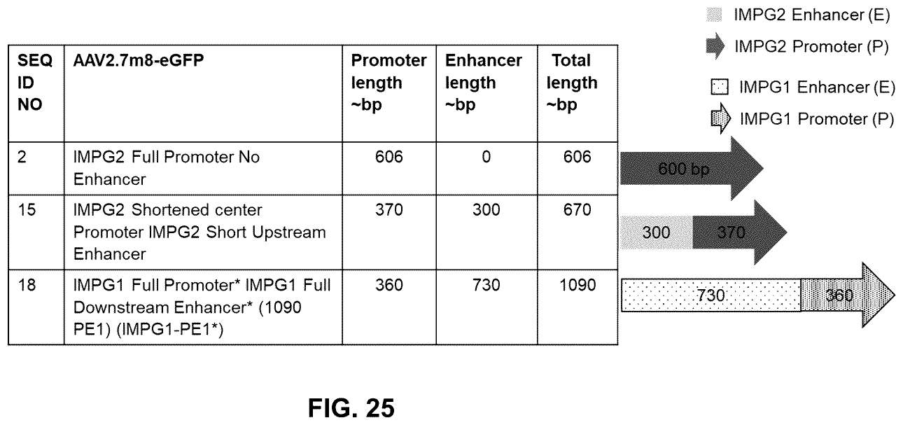

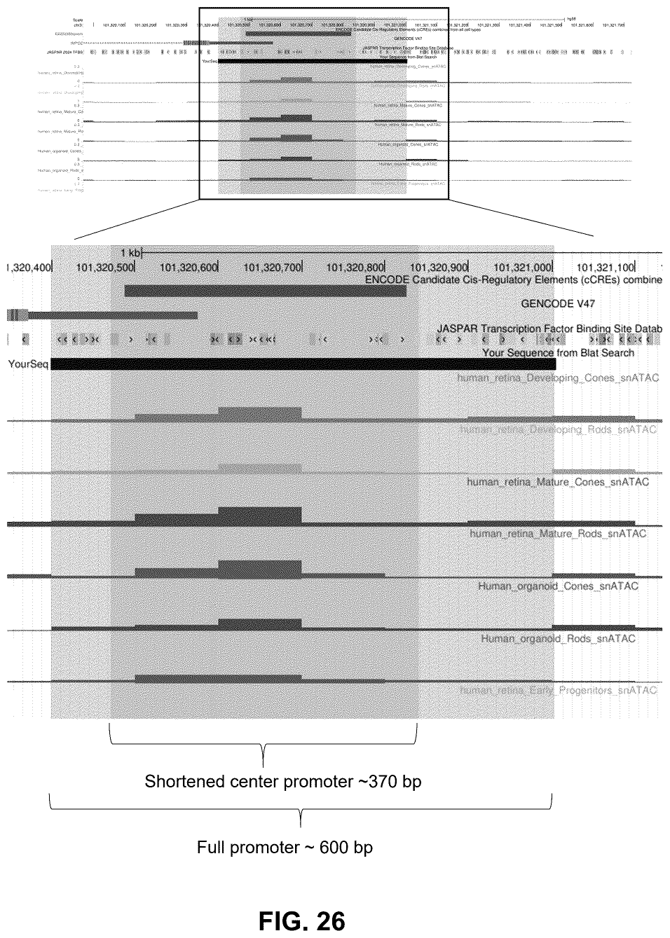

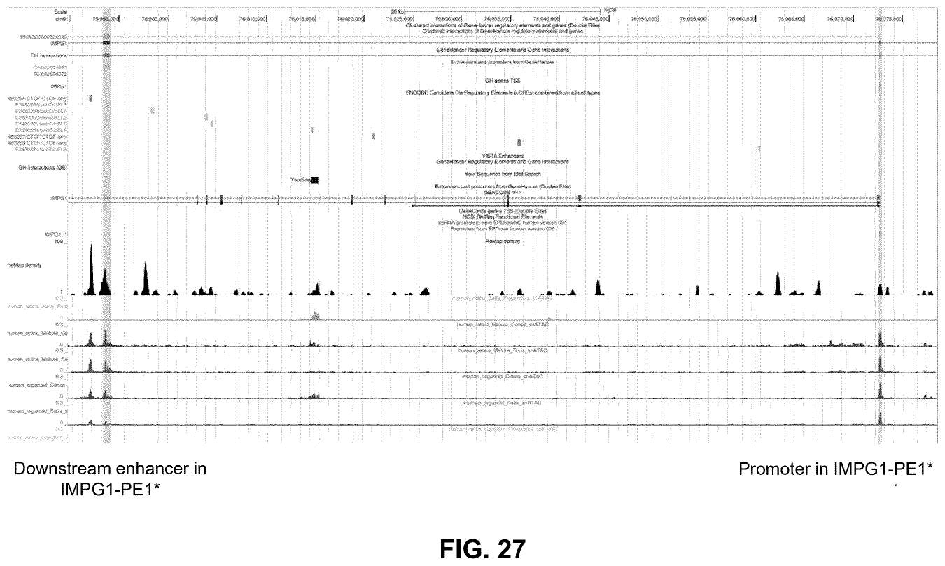

In round 2, three constructs comprising IMPG2 full length promoter without enhancer, IMPG2 short center promoter with IMPG2 short enhancer, and IMPG1 promoter and IMPG 1 downstream enhancer (IMPG1-PE1) were tested (FIG. 25). Since shortening the enhancer did not impact activity of the IMPG2 promoter-enhancer, the IMPG2-no enhancer construct was used to test if the enhancer might be redundant. To test whether a different region of the promoter could be removed, the scIMPG2 promoter was generated that only retained 370 bp in the center region (FIG. 26). Lastly, IMPG1-PE1, which was not expressed at high level and did not show any activity in Y79 cell model, was tested for activity in whole retinal organoid model that might better express IMPG1-PE1 (FIG. 27). The promoter and enhancer sequence of IMPG1-PE1* was different compared to the promoter and enhancer sequence of IMPG1-PE1 that was tested in Y79 cell model in FIG. 6 and FIG. 7. Particularly, the enhancer sequence in IMPG1-PE1* was downstream of the enhancer sequence in IMPG1-PE1 (FIG. 27)

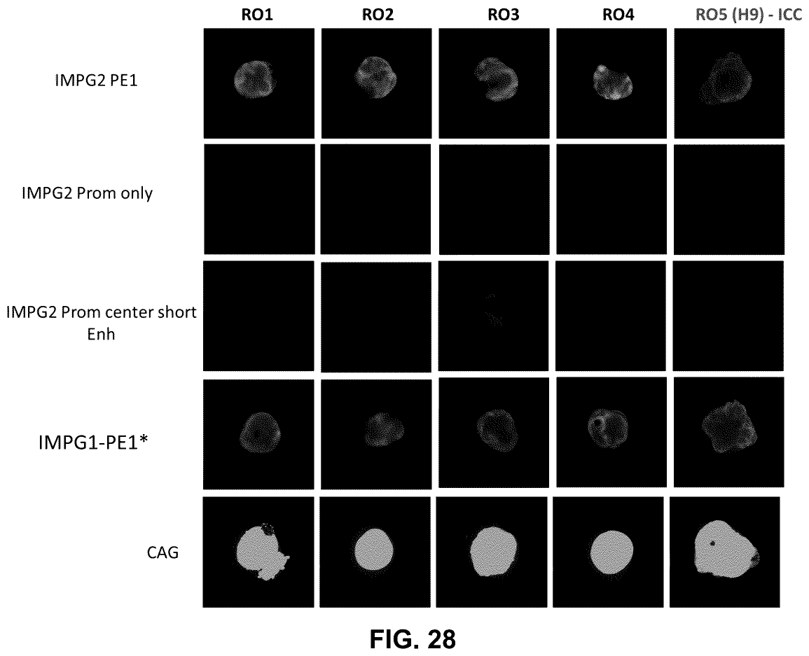

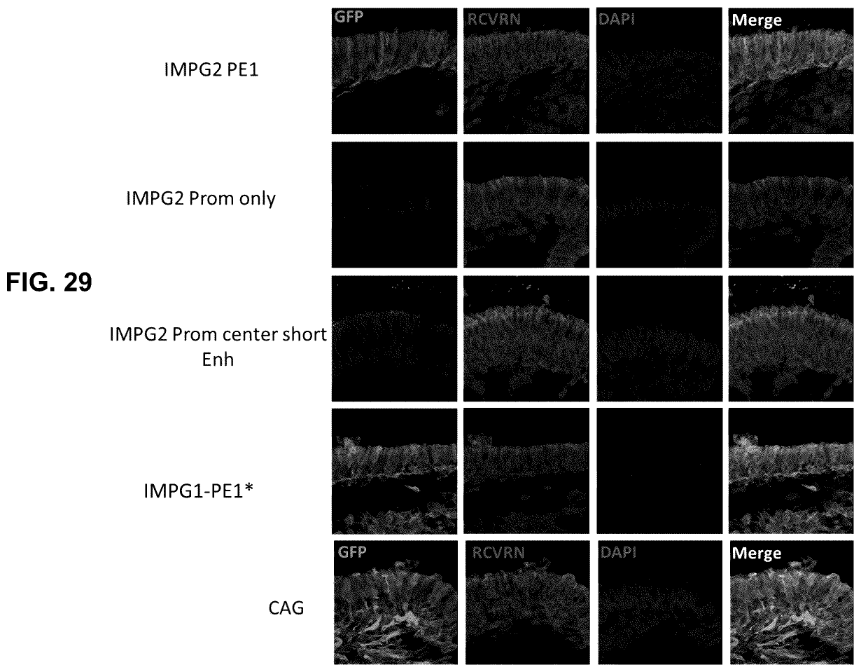

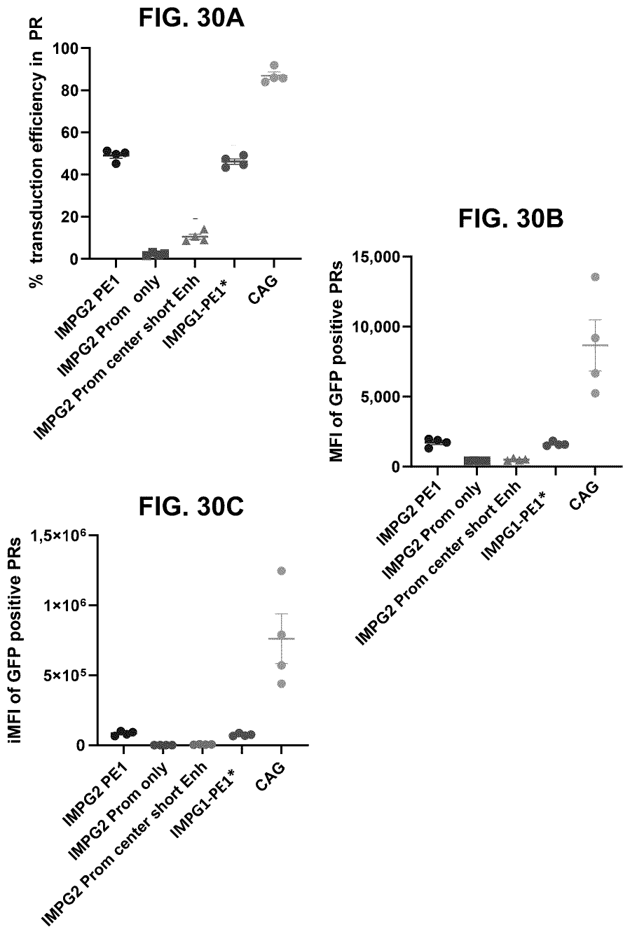

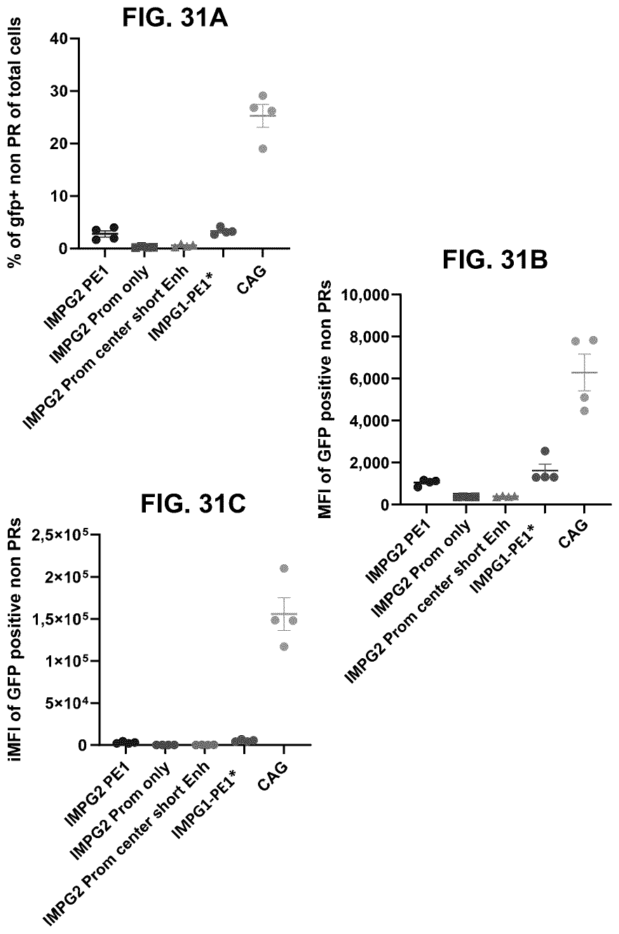

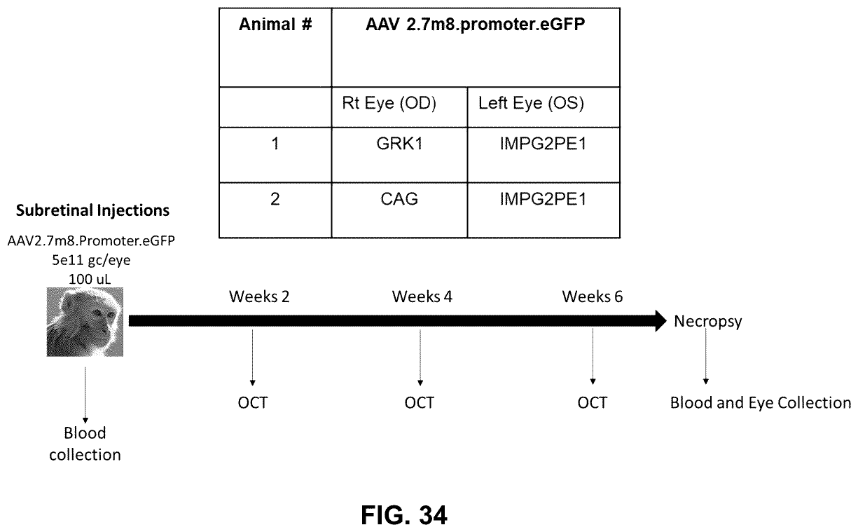

These results showed that IMPG2 promoter only and IMPG2 short center promoter with IMPG2 shorted enhancer constructs had minimum to low expression of GFP in PR or non-PR, indicating that the enhancer and the center region of the IMPG2 promoter were essential for activity, respectively (FIGS. 28-32). Surprisingly, IMPG1-PE1 showed some level of GFP expression (FIG. 28, FIG. 29, and FIGS. 30A-30C) with comparable PR specificity to IMGP2-PE1 (FIG. 30A-30C, FIGS. 31A-31C, and FIG. 34).

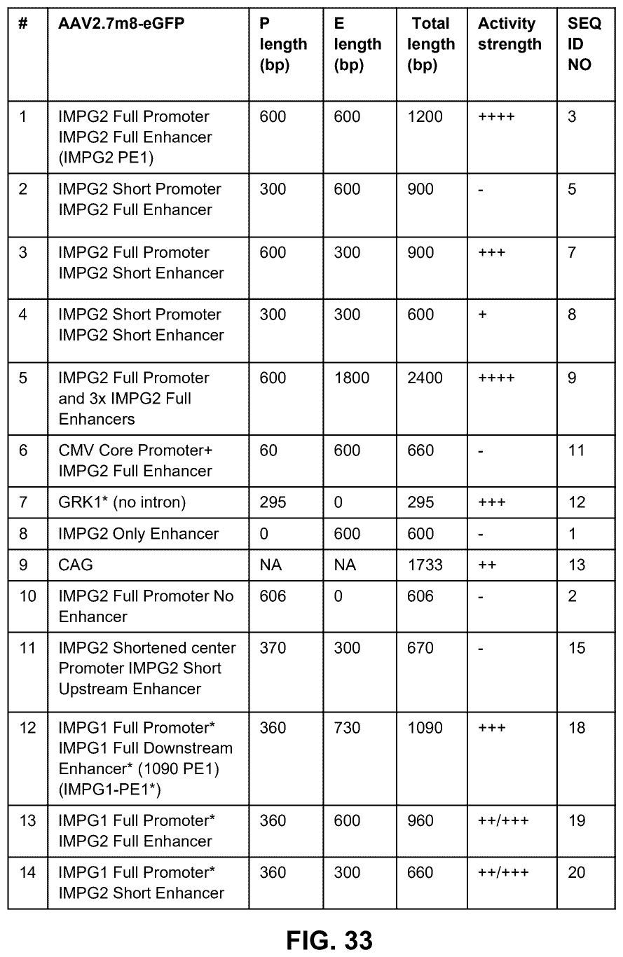

In summary, round 1 and round 2 screens of various lengths of promoter-enhancer identified positive constructs with significant level expression and PR specificity. These constructs included: IMPG2 Full Promoter IMPG2 Full Enhancer (IMPG2-PE1) (SEQ ID NO: 3), IMPG2 Full Promoter IMPG2 Short Enhancer (SEQ ID NO: 7), IMPG2 Short Promoter IMPG2 Short Enhancer (SEQ ID NO: 8), IMPG2 Full Promoter and 3 units of IMPG2 Full Enhancers (SEQ ID NO: 9), and IMPG1 Full Promoter* and IMPG1 Full Enhancer* (IMPG1-PE1*) (SEQ ID NO: 18) (see summary in FIG. 33).

### Example 9

### Round 3: Designing Shortened Promoter-Enhancer Constructs

Round 3 set out to test whether IMPG1 promoter can replace IMPG2 and pair with the full length or shortened enhancer of IMPG2. Based on the experimental results set forth herein, chimeric constructs comprising IMPG1 full promoter* with short enhancer of IMPG2 (SEQ ID NO: 20) or IMPG1 full promoter* with full length enhancer of IMPG2 (SEQ ID NO: 19) can drive the same or comparable level of expression and PR specificity as the positive constructs identified in Round 1 and Round 2 (see summary in FIG. 33).

### Example 10

### IMPG2-PE1 In Vivo Activity in Non-Human Primates (NHPs)

To demonstrate that IMPG2-PE1 could promote expression of a transgene specifically in PR in vivo, AAV2.7m8 vector containing eGFP reporter gene under the regulation of one of the test promoters (IMPG2-PE1 promoter-enhancer, GRK1 promoter, or CAG promoter) were injected into cynomolgus macaques (FIG. 34). Female cynomolgus macaques, 3 years of age and weighing approximately 3 kg, were anesthetized by intramuscular injection of ketamine (2-10 mg/kg), dexmedetomidine (0.025 mg/kg), and glycopyrrolate (0.01 mg/kg) and pupils were dilated with a topical mydriatic agent. Using a trocar, two cannulas (23- or 25-gauge) were placed through the conjunctiva and sclera approximately 3 mm posterior to the corneal limbus, one at the 10 o'clock and the other at the 2 o'clock meridian. A fiberoptic light was passed through the 25-gauge cannulas to illuminate the retina. A plano-convex corneal contact lens was used to visualize the retina through a surgical microscope. A 23-gauge injector (D.O.R.C., Exeter, NH) with a 41-gauge extension was then passed through the 23-gauge cannula and the cannula tip was extended and brought in contact with the retina. The vector (100 μl at a dose of 5×10¹¹ genome copies/eye, wherein the vector was stored in PBS buffer pH7.4 supplemented with 200 mM NaCl and 0.001% pluronic F-68; and diluted in balanced salt solution) was injected through the retina into the subretinal space, resulting in a subretinal bleb. The 41-gauge cannula tip was retracted and the injector tip was repositioned for the second injection. After the second subretinal injection, the instruments and cannulas were removed from the eye. The sclerotomies were cauterized. A topical antibiotic (0.3% tobramycin/0.1% dexamethasone ophthalmic ointment) was instilled in each eye post-injection. To minimize animal-to-animal variability, vectors comprising different promoter/promoter-enhancer constructs were injected in the two eyes of each animal, using a Latin square design so that a total of four eyes were injected with each vector.

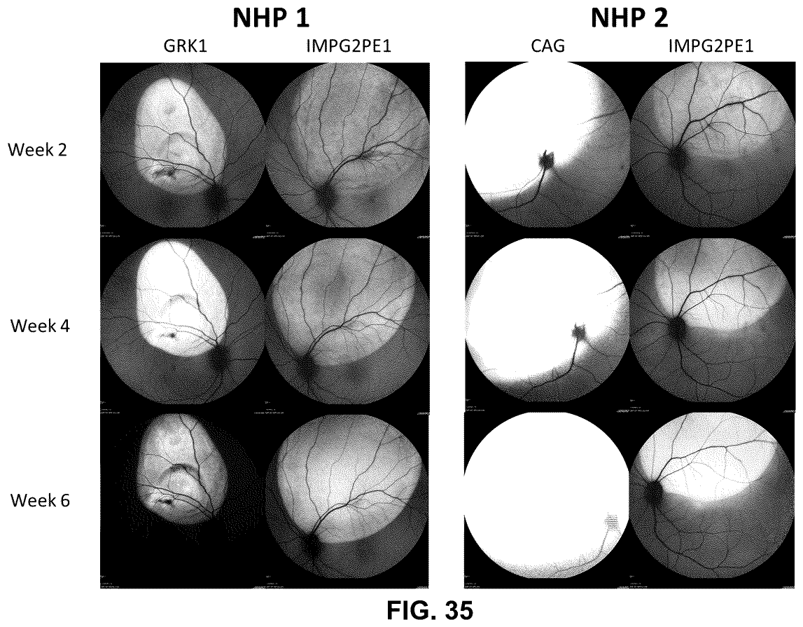

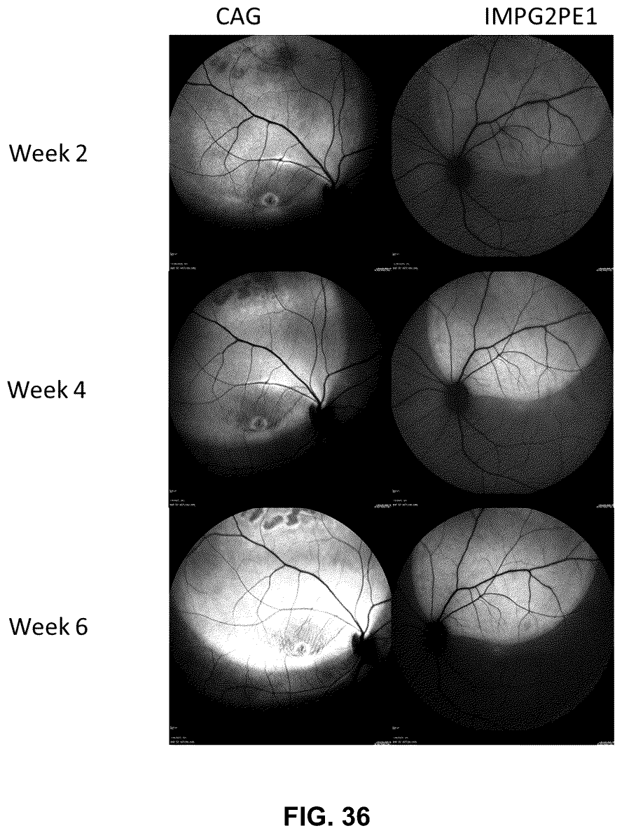

The GFP fluorescence was imaged using Optical Coherence Tomography (OCT) at week 2, week 4, and week 6. FIG. 35 demonstrated that compared to GRK1 (which reached a peak expression by week 4 and decreased by week 6), expression driven by IMPG2-PE1 was lower from week 2 to week 4 but kept increasing slowly by week 6. In contrast, expression driven by CAG was saturated by week 2 and kept spreading throughout the retina by week 6 (FIG. 35 and FIG. 36).

After OCT imaging at week 6, the eyes were enucleated immediately after animal sacrifice, placed into cold (4° C.) 0.1 M phosphate buffer, and promptly placed on a cold plate under a dissecting microscope. Excess tissue was removed and a coronal cut was made to remove the anterior segment (cornea, iris, and lens). Four radial cuts, one in each quadrant, were made through the retina and sclera extending from the peripheral cut edge to just posterior to the equator in order to flatten the posterior segment. Punches of the retina were made in the superior bleb using a 6 mm trephine placed in a specimen tube, frozen and stored at −80° C. for reverse transcription quantitative PCR (RT-qPCR) analysis of transgene expression. The remainder of the eye was placed into 4% paraformaldehyde in phosphate buffer at 4° C. for 2 days and then transferred to 0.1 M phosphate buffer and stored at 4° C. A parasagittal strip of retina/sclera was dissected from each eye that included the blebs, macula, and periphery and embedded in paraffin for immunohistochemistry.

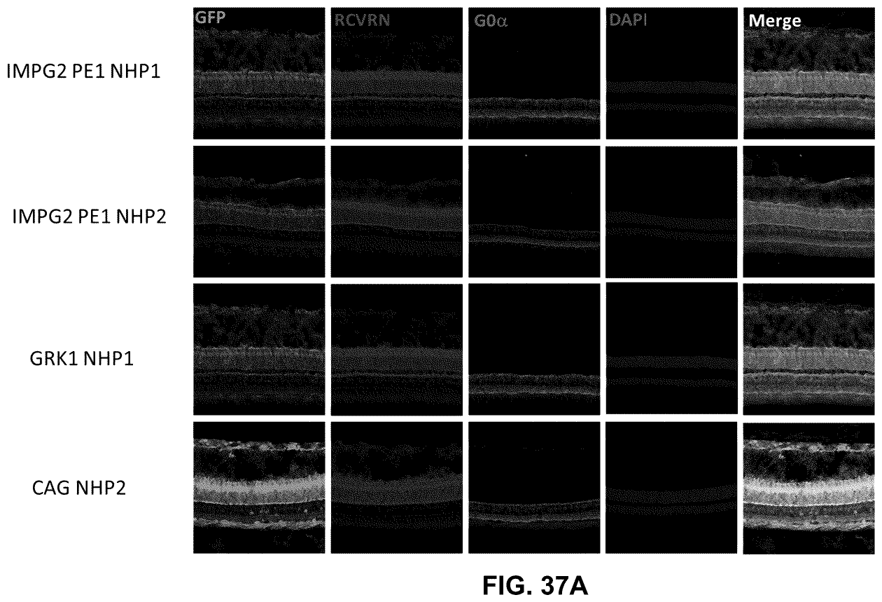

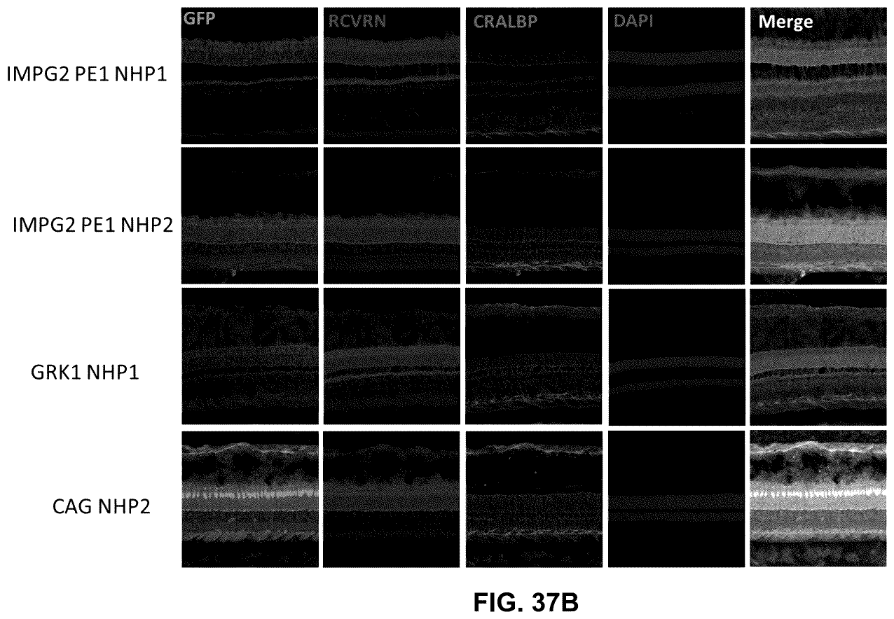

Co-localization of recoverin and GFP indicated that the expression driven by IMPG2-PE1 observed in OCT imaging was specifically from PRs. There was also minimal overlap of GFP expression driven by IMPG2-PE1 with GOα staining (which was a marker for retinal ganglion cells) and with cellular retinaldehyde-binding protein (CRALBP) (which was a marker for RPE) (FIG. 37A and FIG. 37B). In contrast, CAG showed co-localization of GFP with CRALBP, indicating non-PR expression (FIG. 37A and FIG. 37B).

Taken together, the data showed that IMPG2-PE1 was specific to PR and effective in driving robust transgene expression in vivo. The expression was gradual but consistently increasing compared to expression driven by GRK1 which seemed to be reduced between week 4 and week 6. This feature of IMPG2-PE1 could be advantageous in providing long lasting therapeutic benefits brought by expression of the transgene.

### Example 11

### Testing IMPG1-PE1 Construct in Retinal Organoids

The IMPG1-PE1 construct tested in Example 2 in Y79 cells is tested in retinal organoids. FIG. 5 shows that Y79 cells did not express IMPG1 and thus it was not possible to assess the activity of IMPG1-PE1. Based on the experimental results set forth herein, wherein an IMPG1-PE1* construct was functional as shown in FIGS. 28-32. IMPG1-PE1 constructs also function in retinal organoids.

All publications, patents, and patent applications mentioned in this specification are herein incorporated by reference to the same extent as if each individual publication, patent, and patent application was specifically and individually indicated to be incorporated by reference.

While some embodiments have been illustrated and described in detail in the appended drawings and the foregoing description, such illustration and description are to be considered illustrative and not restrictive. Other variations to the disclosed embodiments can be understood and effected in practicing the claims, from a study of the drawings the disclosure, and the appended claims. The mere fact that certain measures or features are recited in mutually different dependent claims does not indicate that the combination of these measures or features cannot be used. Any reference signs in the claims should not be construed as limiting the scope.

SEQUENCE LISTING

| SEQ ID | | |
|---|---|---|
| NO | SEQUENCE | DESCRIPTION |
| 1 | TCCTATTGTTAACTTTGAACTTCCAATTCATTTGGAG | IMPG2 Full |
| | AAGTGGTTTCCTCGTCATGTAATAAAGACCAAACGG | Upstream |
| | GAAGATATTCGAGAGCTCATCTTGAAACTTCATTTA | Enhancer (E1) |
| | CAGCAACAGAAGGGCAGCAGTAGCTAGTTAATCTGT | |
| | ACAAACATGACACAGATGTTCTCTAAGATTACTGGA | |
| | AAGCCTCTTACCAGCATTTGTGTTAGCCAGCTCACA | |
| | GAGAAGAAAATAACTTGCAGTAGTTTTATAATAAGT | |
| | CATTGGAACATTATTTAAAATATGTAGGACACATTA | |
| | TTAGAATTGTTGGGATCTCATAGATGGAATGGGAAT | |
| | GGGGGTGATATAGATAAACTTACTAGATATAAATTA | |
| | AAATTTTATAAATATTTCATATTTTTCTGAGTAAATA | |
| | TGATTGGATTATGCAACAGCATATGTAATATGGGAA | |
| | TGTTTTGTAGATAATAAAACTTACATGATCTGTACTT | |
| | CCACGTGACTGGGTGCTGAGGGGAGTTAAAGCCTCC | |
| | CTGGTGCCAGCCCCAGTGCTTGTCAAATTTGCTGAC | |
| | AGGTCACATCATATTGTAATTCTATTCTTTGCAGCTC | |
| | AAGCATGCAGTA | |
| 2 | AGTTAATTAGTTAATTTAATCCTCACATTAGCTCTAC | IMPG2 Full |
| | CATGAGTTTACTATTTCTATTCCATTTTATACGTAAG | Promoter |
| | GAAGGAGACAAAGTAAGTGATTTTTCTATCAAGGAA | |
| | GGAAATTTGCAAGAGAATAGTTTCATTACAAAAACT | |
| | AAATTTGTACGTAGCTCTGTATTATTGAAATAGGTA | |
| | GATATAGTCAGTCTGGACTTTTTATGCTTATACATCT | |
| | TAGTCCCTAGGAAAACCCAGAACTAACAGATTCAGA | |
| | AAAGTTGGAAAAATCAGTGAATTATATGTGAAACAC | |
| | ATTATTCTTAGTGGACTGCTTGTTAAAGGCAAGGAG | |
| | AGTGTTAGTAAAGAGCTTAGGTAGATTAGAATAAAG | |
| | AAATTGTCTCTCTCCATCTGCTCTAATTAGCTTATCT | |
| | CACCAGCTTTTATAGCATGCTGGTTATTTCAGAAAA | |
| | GAAGTGAGAGCTACTTTGAAAGGACAACCATTTTTC | |
| | TTTCCGCTAATTTATAATGGTTTTGAAGTGGTTGTTC | |
| | ATTCTCAAACATAGACTTTTAAATGTTAGGTCTTTCC | |
| | TATAACTCTTTGTTATTGGAAGTTTCAAGGATTTGGA | |
| | CACTCAATTAAGGATTCTGTCCT | |
| 3 | TCCTATTGTTAACTTTGAACTTCCAATTCATTTGGAG | IMPG2 Full |
| | AAGTGGTTTCCTCGTCATGTAATAAAGACCAAACGG | Promoter And |
| | GAAGATATTCGAGAGCTCATCTTGAAACTTCATTTA | IMPG2 Full |
| | CAGCAACAGAAGGGCAGCAGTAGCTAGTTAATCTGT | Upstream |
| | ACAAACATGACACAGATGTTCTCTAAGATTACTGGA | enhancer |
| | AAGCCTCTTACCAGCATTTGTGTTAGCCAGCTCACA | (IMPG2-PE1) |
| | GAGAAGAAAATAACTTGCAGTAGTTTTATAATAAGT | |
| | CATTGGAACATTATTTAAAATATGTAGGACACATTA | |
| | TTAGAATTGTTGGGATCTCATAGATGGAATGGGAAT | |
| | GGGGGTGATATAGATAAACTTACTAGATATAAATTA | |
| | AAATTTTATAAATATTTCATATTTTTCTGAGTAAATA | |
| | TGATTGGATTATGCAACAGCATATGTAATATGGGAA | |
| | TGTTTTGTAGATAATAAAACTTACATGATCTGTACTT | |
| | CCACGTGACTGGGTGCTGAGGGGAGTTAAAGCCTCC | |
| | CTGGTGCCAGCCCCAGTGCTTGTCAAATTTGCTGAC | |
| | AGGTCACATCATATTGTAATTCTATTCTTTGCAGCTC | |
| | AAGCATGCAGTAAGTTAATTAGTTAATTTAATCCTC | |
| | ACATTAGCTCTACCATGAGTTTACTATTTCTATTCCA | |
| | TTTTATACGTAAGGAAGGAGACAAAGTAAGTGATTT | |
| | TTCTATCAAGGAAGGAAATTTGCAAGAGAATAGTTT | |
| | CATTACAAAAACTAAATTTGTACGTAGCTCTGTATTA | |
| | TTGAAATAGGTAGATATAGTCAGTCTGGACTTTTTAT | |
| | GCTTATACATCTTAGTCCCTAGGAAAACCCAGAACT | |
| | AACAGATTCAGAAAAGTTGGAAAAATCAGTGAATTA | |
| | TATGTGAAACACATTATTCTTAGTGGACTGCTTGTTA | |
| | AAGGCAAGGAGAGTGTTAGTAAAGAGCTTAGGTAG | |
| | ATTAGAATAAAGAAATTGTCTCTCTCCATCTGCTCTA | |
| | ATTAGCTTATCTCACCAGCTTTTATAGCATGCTGGTT | |
| | ATTTCAGAAAAGAAGTGAGAGCTACTTTGAAAGGAC | |
| | AACCATTTTTCTTTCCGCTAATTTATAATGGTTTTGA | |
| | AGTGGTTGTTCATTCTCAAACATAGACTTTTAAATGT | |
| | TAGGTCTTTCCTATAACTCTTTGTTATTGGAAGTTTC | |
| | AAGGATTTGGACACTCAATTAAGGATTCTGTCCT | |
| 4 | GACTGCTTGTTAAAGGCAAGGAGAGTGTTAGTAAAG | IMPG2 Short |
| | AGCTTAGGTAGATTAGAATAAAGAAATTGTCTCTCT | Promoter |
| | CCATCTGCTCTAATTAGCTTATCTCACCAGCTTTTAT | |
| | AGCATGCTGGTTATTTCAGAAAAGAAGTGAGAGCTA | |
| | CTTTGAAAGGACAACCATTTTTCTTTCCGCTAATTTA | |
| | TAATGGTTTTGAAGTGGTTGTTCATTCTCAAACATAG | |
| | ACTTTTAAATGTTAGGTCTTTCCTATAACTCTTTGTT | |
| | ATTGGAAGTTTCAAGGATTTGGACACTCAATTAAGG | |
| | ATTCTGTCCT | |
| 5 | TCCTATTGTTAACTTTGAACTTCCAATTCATTTGGAG | IMPG2 Short |
| | AAGTGGTTTCCTCGTCATGTAATAAAGACCAAACGG | Promoter And |
| | GAAGATATTCGAGAGCTCATCTTGAAACTTCATTTA | IMPG2 Full |
| | CAGCAACAGAAGGGCAGCAGTAGCTAGTTAATCTGT | Upstream |
| | ACAAACATGACACAGATGTTCTCTAAGATTACTGGA | Enhancer |
| | AAGCCTCTTACCAGCATTTGTGTTAGCCAGCTCACA | |
| | GAGAAGAAAATAACTTGCAGTAGTTTTATAATAAGT | |
| | CATTGGAACATTATTTAAAATATGTAGGACACATTA | |
| | TTAGAATTGTTGGGATCTCATAGATGGAATGGGAAT | |
| | GGGGGTGATATAGATAAACTTACTAGATATAAATTA | |
| | AAATTTTATAAATATTTCATATTTTTCTGAGTAAATA | |
| | TGATTGGATTATGCAACAGCATATGTAATATGGGAA | |
| | TGTTTTGTAGATAATAAAACTTACATGATCTGTACTT | |
| | CCACGTGACTGGGTGCTGAGGGGAGTTAAAGCCTCC | |
| | CTGGTGCCAGCCCCAGTGCTTGTCAAATTTGCTGAC | |
| | AGGTCACATCATATTGTAATTCTATTCTTTGCAGCTC | |
| | AAGCATGCAGTAGACTGCTTGTTAAAGGCAAGGAGA | |
| | GTGTTAGTAAAGAGCTTAGGTAGATTAGAATAAAGA | |
| | AATTGTCTCTCTCCATCTGCTCTAATTAGCTTATCTC | |
| | ACCAGCTTTTATAGCATGCTGGTTATTTCAGAAAAG | |
| | AAGTGAGAGCTACTTTGAAAGGACAACCATTTTTCT | |
| | TTCCGCTAATTTATAATGGTTTTGAAGTGGTTGTTCA | |
| | TTCTCAAACATAGACTTTTAAATGTTAGGTCTTTCCT | |
| | ATAACTCTTTGTTATTGGAAGTTTCAAGGATTTGGAC | |
| | ACTCAATTAAGGATTCTGTCCT | |
| 6 | TCCTATTGTTAACTTTGAACTTCCAATTCATTTGGAG | IMPG2 Short |
| | AAGTGGTTTCCTCGTCATGTAATAAAGACCAAACGG | Upstream |
| | GAAGATATTCGAGAGCTCATCTTGAAACTTCATTTA | Enhancer 1 |
| | CAGCAACAGAAGGGCAGCAGTAGCTAGTTAATCTGT | |
| | ACAAACATGACACAGATGTTCTCTAAGATTACTGGA | |
| | AAGCCTCTTACCAGCATTTGTGTTAGCCAGCTCACA | |
| | GAGAAGAAAATAACTTGCAGTAGTTTTATAATAAGT | |
| | CATTGGAACATTATTTAAAATATGTAGGACACATTA | |
| | TTAGAATTGTT | |
| 7 | TCCTATTGTTAACTTTGAACTTCCAATTCATTTGGAG | IMPG2 Full |
| | AAGTGGTTTCCTCGTCATGTAATAAAGACCAAACGG | Promoter And |
| | GAAGATATTCGAGAGCTCATCTTGAAACTTCATTTA | IMPG2 Short |
| | CAGCAACAGAAGGGCAGCAGTAGCTAGTTAATCTGT | Upstream |
| | ACAAACATGACACAGATGTTCTCTAAGATTACTGGA | Enhancer |
| | AAGCCTCTTACCAGCATTTGTGTTAGCCAGCTCACA | |
| | GAGAAGAAAATAACTTGCAGTAGTTTTATAATAAGT | |
| | CATTGGAACATTATTTAAAATATGTAGGACACATTA | |
| | TTAGAATTGTTAGTTAATTAGTTAATTTAATCCTCAC | |
| | ATTAGCTCTACCATGAGTTTACTATTTCTATTCCATT | |
| | TTATACGTAAGGAAGGAGACAAAGTAAGTGATTTTT | |
| | CTATCAAGGAAGGAAATTTGCAAGAGAATAGTTTCA | |
| | TTACAAAAACTAAATTTGTACGTAGCTCTGTATTATT | |
| | GAAATAGGTAGATATAGTCAGTCTGGACTTTTTATG | |
| | CTTATACATCTTAGTCCCTAGGAAAACCCAGAACTA | |
| | ACAGATTCAGAAAAGTTGGAAAAATCAGTGAATTAT | |
| | ATGTGAAACACATTATTCTTAGTGGACTGCTTGTTAA | |
| | AGGCAAGGAGAGTGTTAGTAAAGAGCTTAGGTAGA | |
| | TTAGAATAAAGAAATTGTCTCTCTCCATCTGCTCTAA | |
| | TTAGCTTATCTCACCAGCTTTTATAGCATGCTGGTTA | |
| | TTTCAGAAAAGAAGTGAGAGCTACTTTGAAAGGACA | |
| | ACCATTTTTCTTTCCGCTAATTTATAATGGTTTTGAA | |
| | GTGGTTGTTCATTCTCAAACATAGACTTTTAAATGTT | |
| | AGGTCTTTCCTATAACTCTTTGTTATTGGAAGTTTCA | |
| | AGGATTTGGACACTCAATTAAGGATTCTGTCCT | |
| 8 | TCCTATTGTTAACTTTGAACTTCCAATTCATTTGGAG | IMPG2 Short |
| | AAGTGGTTTCCTCGTCATGTAATAAAGACCAAACGG | Promoter And |
| | GAAGATATTCGAGAGCTCATCTTGAAACTTCATTTA | IMPG2 Short |
| | CAGCAACAGAAGGGCAGCAGTAGCTAGTTAATCTGT | Upstream |
| | ACAAACATGACACAGATGTTCTCTAAGATTACTGGA | Enhancer |
| | AAGCCTCTTACCAGCATTTGTGTTAGCCAGCTCACA | |
| | GAGAAGAAAATAACTTGCAGTAGTTTTATAATAAGT | |
| | CATTGGAACATTATTTAAAATATGTAGGACACATTA | |
| | TTAGAATTGTTGACTGCTTGTTAAAGGCAAGGAGAG | |
| | TGTTAGTAAAGAGCTTAGGTAGATTAGAATAAAGAA | |
| | ATTGTCTCTCTCCATCTGCTCTAATTAGCTTATCTCA | |
| | CCAGCTTTTATAGCATGCTGGTTATTTCAGAAAAGA | |
| | AGTGAGAGCTACTTTGAAAGGACAACCATTTTTCTTT | |
| | CCGCTAATTTATAATGGTTTTGAAGTGGTTGTTCATT | |
| | CTCAAACATAGACTTTTAAATGTTAGGTCTTTCCTAT | |
| | AACTCTTTGTTATTGGAAGTTTCAAGGATTTGGACAC | |
| | TCAATTAAGGATTCTGTCCT | |
| 9 | TCCTATTGTTAACTTTGAACTTCCAATTCATTTGGAG | IMPG2 Full |
| | AAGTGGTTTCCTCGTCATGTAATAAAGACCAAACGG | Promoter And 3x |
| | GAAGATATTCGAGAGCTCATCTTGAAACTTCATTTA | IMPG2 Full |
| | CAGCAACAGAAGGGCAGCAGTAGCTAGTTAATCTGT | Upstream |
| | ACAAACATGACACAGATGTTCTCTAAGATTACTGGA | Enhancer |
| | AAGCCTCTTACCAGCATTTGTGTTAGCCAGCTCACA | |
| | GAGAAGAAAATAACTTGCAGTAGTTTTATAATAAGT | |
| | CATTGGAACATTATTTAAAATATGTAGGACACATTA | |
| | TTAGAATTGTTGGGATCTCATAGATGGAATGGGAAT | |
| | GGGGGTGATATAGATAAACTTACTAGATATAAATTA | |
| | AAATTTTATAAATATTTCATATTTTTCTGAGTAAATA | |
| | TGATTGGATTATGCAACAGCATATGTAATATGGGAA | |
| | TGTTTTGTAGATAATAAAACTTACATGATCTGTACTT | |
| | CCACGTGACTGGGTGCTGAGGGGAGTTAAAGCCTCC | |
| | CTGGTGCCAGCCCCAGTGCTTGTCAAATTTGCTGAC | |
| | AGGTCACATCATATTGTAATTCTATTCTTTGCAGCTC | |
| | AAGCATGCAGTATCCTATTGTTAACTTTGAACTTCCA | |
| | ATTCATTTGGAGAAGTGGTTTCCTCGTCATGTAATAA | |
| | AGACCAAACGGGAAGATATTCGAGAGCTCATCTTGA | |
| | AACTTCATTTACAGCAACAGAAGGGCAGCAGTAGCT | |
| | AGTTAATCTGTACAAACATGACACAGATGTTCTCTA | |
| | AGATTACTGGAAAGCCTCTTACCAGCATTTGTGTTA | |
| | GCCAGCTCACAGAGAAGAAAATAACTTGCAGTAGTT | |
| | TTATAATAAGTCATTGGAACATTATTTAAAATATGTA | |
| | GGACACATTATTAGAATTGTTGGGATCTCATAGATG | |
| | GAATGGGAATGGGGGTGATATAGATAAACTTACTAG | |
| | ATATAAATTAAAATTTTATAAATATTTCATATTTTTC | |
| | TGAGTAAATATGATTGGATTATGCAACAGCATATGT | |
| | AATATGGGAATGTTTTGTAGATAATAAAACTTACAT | |
| | GATCTGTACTTCCACGTGACTGGGTGCTGAGGGGAG | |
| | TTAAAGCCTCCCTGGTGCCAGCCCCAGTGCTTGTCA | |
| | AATTTGCTGACAGGTCACATCATATTGTAATTCTATT | |
| | CTTTGCAGCTCAAGCATGCAGTATCCTATTGTTAACT | |
| | TTGAACTTCCAATTCATTTGGAGAAGTGGTTTCCTCG | |
| | TCATGTAATAAAGACCAAACGGGAAGATATTCGAGA | |
| | GCTCATCTTGAAACTTCATTTACAGCAACAGAAGGG | |
| | CAGCAGTAGCTAGTTAATCTGTACAAACATGACACA | |
| | GATGTTCTCTAAGATTACTGGAAAGCCTCTTACCAG | |
| | CATTTGTGTTAGCCAGCTCACAGAGAAGAAAATAAC | |
| | TTGCAGTAGTTTTATAATAAGTCATTGGAACATTATT | |
| | TAAAATATGTAGGACACATTATTAGAATTGTTGGGA | |
| | TCTCATAGATGGAATGGGAATGGGGGTGATATAGAT | |
| | AAACTTACTAGATATAAATTAAAATTTTATAAATATT | |
| | TCATATTTTTCTGAGTAAATATGATTGGATTATGCAA | |
| | CAGCATATGTAATATGGGAATGTTTTGTAGATAATA | |
| | AAACTTACATGATCTGTACTTCCACGTGACTGGGTG | |
| | CTGAGGGGAGTTAAAGCCTCCCTGGTGCCAGCCCCA | |
| | GTGCTTGTCAAATTTGCTGACAGGTCACATCATATTG | |
| | TAATTCTATTCTTTGCAGCTCAAGCATGCAGTAAGTT | |
| | AATTAGTTAATTTAATCCTCACATTAGCTCTACCATG | |
| | AGTTTACTATTTCTATTCCATTTTATACGTAAGGAAG | |
| | GAGACAAAGTAAGTGATTTTTCTATCAAGGAAGGAA | |
| | ATTTGCAAGAGAATAGTTTCATTACAAAAACTAAAT | |
| | TTGTACGTAGCTCTGTATTATTGAAATAGGTAGATAT | |
| | AGTCAGTCTGGACTTTTTATGCTTATACATCTTAGTC | |
| | CCTAGGAAAACCCAGAACTAACAGATTCAGAAAAG | |
| | TTGGAAAAATCAGTGAATTATATGTGAAACACATTA | |
| | TTCTTAGTGGACTGCTTGTTAAAGGCAAGGAGAGTG | |
| | TTAGTAAAGAGCTTAGGTAGATTAGAATAAAGAAAT | |
| | TGTCTCTCTCCATCTGCTCTAATTAGCTTATCTCACC | |
| | AGCTTTTATAGCATGCTGGTTATTTCAGAAAAGAAG | |
| | TGAGAGCTACTTTGAAAGGACAACCATTTTTCTTTCC | |
| | GCTAATTTATAATGGTTTTGAAGTGGTTGTTCATTCT | |
| | CAAACATAGACTTTTAAATGTTAGGTCTTTCCTATAA | |
| | CTCTTTGTTATTGGAAGTTTCAAGGATTTGGACACTC | |
| | AATTAAGGATTCTGTCCT | |
| 10 | TAGGCGTGTACGGTGGGAGGTCTATATAAGCAGAGC | CMV Core |
| | TCGTTTAGTGAACC | Promoter |
| 11 | TCCTATTGTTAACTTTGAACTTCCAATTCATTTGGAG | CMV Core |
| | AAGTGGTTTCCTCGTCATGTAATAAAGACCAAACGG | Promoter |
| | GAAGATATTCGAGAGCTCATCTTGAAACTTCATTTA | IMPG2 Full |
| | CAGCAACAGAAGGGCAGCAGTAGCTAGTTAATCTGT | Upstream |
| | ACAAACATGACACAGATGTTCTCTAAGATTACTGGA | Enhancer |
| | AAGCCTCTTACCAGCATTTGTGTTAGCCAGCTCACA | |
| | GAGAAGAAAATAACTTGCAGTAGTTTTATAATAAGT | |
| | CATTGGAACATTATTTAAAATATGTAGGACACATTA | |
| | TTAGAATTGTTGGGATCTCATAGATGGAATGGGAAT | |
| | GGGGGTGATATAGATAAACTTACTAGATATAAATTA | |
| | AAATTTTATAAATATTTCATATTTTTCTGAGTAAATA | |
| | TGATTGGATTATGCAACAGCATATGTAATATGGGAA | |
| | TGTTTTGTAGATAATAAAACTTACATGATCTGTACTT | |
| | CCACGTGACTGGGTGCTGAGGGGAGTTAAAGCCTCC | |
| | CTGGTGCCAGCCCCAGTGCTTGTCAAATTTGCTGAC | |
| | AGGTCACATCATATTGTAATTCTATTCTTTGCAGCTC | |
| | AAGCATGCAGTATAGGCGTGTACGGTGGGAGGTCTA | |
| | TATAAGCAGAGCT CGTTTAGTGAACC | |
| 12 | GGGCCCCAGAAGCCTGGTGGTTGTTTGTCCTTCTCAG | GRK1 Promoter |
| | GGGAAAAGTGAGGCGGCCCCTTGGAGGAAGGGGCC | |
| | GGGCAGAATGATCTAATCGGATTCCAAGCAGCTCAG | |
| | GGGATTGTCTTTTTCTAGCACCTTCTTGCCACTCCTA | |
| | AGCGTCCTCCGTGACCCCGGCTGGGATTTAGCCTGG | |
| | TGCTGTGTCAGCCCCGGGCTCCCAGGGGCTTCCCAG | |
| | TGGTCCCCAGGGAACCCTCGACAGGGCCAGGGCGTC | |
| | TCTCTCGTCCAGCAAGGGCAGGGACGGGCCACAGGC | |
| | AAGGGC | |
| 13 | CTCGACATTGATTATTGACTAGTTATTAATAGTAATC | CAG Promoter |
| | AATTACGGGGTCATTAGTTCATAGCCCATATATGGA | |
| | GTTCCGCGTTACATAACTTACGGTAAATGGCCCGCC | |
| | TGGCTGACCGCCCAACGACCCCCGCCCATTGACGTC | |
| | AATAATGACGTATGTTCCCATAGTAACGCCAATAGG | |
| | GACTTTCCATTGACGTCAATGGGTGGAGTATTTACG | |
| | GTAAACTGCCCACTTGGCAGTACATCAAGTGTATCA | |
| | TATGCCAAGTACGCCCCCTATTGACGTCAATGACGG | |
| | TAAATGGCCCGCCTGGCATTATGCCCAGTACATGAC | |
| | CTTATGGGACTTTCCTACTTGGCAGTACATCTACGTA | |
| | TTAGTCATCGCTATTACCATGGTCGAGGTGAGCCCC | |
| | ACGTTCTGCTTCACTCTCCCCATCTCCCCCCCCTCCC | |
| | CACCCCCAATTTTGTATTTATTTATTTTTTAATTATTT | |
| | TGTGCAGCGATGGGGGCGGGGGGGGGGGGGGGGCG | |
| | CGCGCCAGGCGGGGCGGGGCGGGGCGAGGGGGGGG | |
| | GCGGGGCGAGGCGGAGAGGTGCGGCGGCAGCCAAT | |
| | CAGAGCGGCGCGCTCCGAAAGTTTCCTTTTATGGCG | |
| | AGGCGGCGGCGGCGGCGGCCCTATAAAAAGCGAAG | |
| | CGCGCGGCGGGCGGGAGTCGCTGCGCGCTGCCTTCG | |
| | CCCCGTGCCCCGCTCCGCCGCCGCCTCGCGCCGCCC | |
| | GCCCCGGCTCTGACTGACCGCGTTACTCCCACAGGT | |
| | GAGCGGGCGGGACGGCCCTTCTCCTCCGGGCTGTAA | |
| | TTAGCGCTTGGTTTAATGACGGCTTGTTTCTTTTCTG | |
| | TGGCTGCGTGAAAGCCTTGAGGGGCTCCGGGAGGGC | |
| | CCTTTGTGCGGGGGGAGCGGCTCGGGGGGTGCGTGC | |
| | GTGTGTGTGTGCGTGGGGAGCGCCGCGTGCGGCTCC | |
| | GCGCTGCCCGGCGGCTGTGAGCGCTGCGGGCGCGGC | |
| | GCGGGGCTTTGTGCGCTCCGCAGTGTGCGCGAGGGG | |
| | AGCGCGGCCGGGGGCGGTGCCCCGCGGTGCGGGGG | |
| | GGGCTGCGAGGGGAACAAAGGCTGCGTGCGGGGTG | |
| | TGTGCGTGGGGGGGTGAGCAGGGGGTGTGGGCGCG | |
| | TCGGTCGGGCTGCAACCCCCCCTGCACCCCCCTCCCC | |
| | GAGTTGCTGAGCACGGCCCGGCTTCGGGTGCGGGGC | |
| | TCCGTACGGGGCGTGGCGCGGGGCTCGCCGTGCCGG | |
| | GCGGGGGGTGGCGGCAGGTGGGGGTGCCGGGCGGG | |
| | GCGGGGCCGCCTCGGGCCGGGGAGGGCTCGGGGGA | |
| | GGGGCGCGGCGGCCCCCGGAGCGCCGGCGGCTGTC | |
| | GAGGCGCGGCGAGCCGCAGCCATTGCCTTTTATGGT | |
| | AATCGTGCGAGAGGGCGCAGGGACTTCCTTTGTCCC | |
| | AAATCTGTGCGGAGCCGAAATCTGGGAGGCGCCGCC | |
| | GCACCCCCTCTAGCGGGCGCGGGGCGAAGCGGTGCG | |
| | GCGCCGGCAGGAAGGAAATGGGGGGGGAGGGCCTT | |
| | CGTGCGTCGCCGCGCCGCCGTCCCCTTCTCCCTCTCC | |
| | AGCCTCGGGGCTGTCCGCGGGGGGACGGCTGCCTTC | |
| | GGGGGGGACGGGGCAGGGCGGGGTTCGGCTTCTGG | |
| | CGTGTGACCGGCGGCTCTAGAGCCTCTGCTAACCAT | |
| | GTTCATGCCTTCTTCTTTTTCCTACAGCTCCTGGGCA | |
| | ACGTGCTGGTTATTGTGCTGTCTCATCATTTTGGCAA | |
| | AGAATTG | |
| 14 | TATTATTGAAATAGGTAGATATAGTCAGTCTGGACT | IMPG2 Short |
| | TTTTATGCTTATACATCTTAGTCCCTAGGAAAACCCA | Center Promoter |
| | GAACTAACAGATTCAGAAAAGTTGGAAAAATCAGT | |
| | GAATTATATGTGAAACACATTATTCTTAGTGGACTG | |
| | CTTGTTAAAGGCAAGGAGAGTGTTAGTAAAGAGCTT | |
| | AGGTAGATTAGAATAAAGAAATTGTCTCTCTCCATC | |
| | TGCTCTAATTAGCTTATCTCACCAGCTTTTATAGCAT | |
| | GCTGGTTATTTCAGAAAAGAAGTGAGAGCTACTTTG | |
| | AAAGGACAACCATTTTTCTTTCCGCTAATTTATAATG | |
| | GTTTTGAAGTGGTTGTTCATTCTCAAACATAGACTTT | |
| | TAAATGT | |
| 15 | TCCTATTGTTAACTTTGAACTTCCAATTCATTTGGAG | IMPG2 Short |
| | AAGTGGTTTCCTCGTCATGTAATAAAGACCAAACGG | Center Promoter |
| | GAAGATATTCGAGAGCTCATCTTGAAACTTCATTTA | And IMPG2 |
| | CAGCAACAGAAGGGCAGCAGTAGCTAGTTAATCTGT | Short Upstream |
| | ACAAACATGACACAGATGTTCTCTAAGATTACTGGA | Enhancer |
| | AAGCCTCTTACCAGCATTTGTGTTAGCCAGCTCACA | |
| | GAGAAGAAAATAACTTGCAGTAGTTTTATAATAAGT | |
| | CATTGGAACATTATTTAAAATATGTAGGACACATTA | |
| | TTAGAATTGTTTATTATTGAAATAGGTAGATATAGTC | |
| | AGTCTGGACTTTTTATGCTTATACATCTTAGTCCCTA | |
| | GGAAAACCCAGAACTAACAGATTCAGAAAAGTTGG | |
| | AAAAATCAGTGAATTATATGTGAAACACATTATTCT | |
| | TAGTGGACTGCTTGTTAAAGGCAAGGAGAGTGTTAG | |
| | TAAAGAGCTTAGGTAGATTAGAATAAAGAAATTGTC | |
| | TCTCTCCATCTGCTCTAATTAGCTTATCTCACCAGCT | |
| | TTTATAGCATGCTGGTTATTTCAGAAAAGAAGTGAG | |
| | AGCTACTTTGAAAGGACAACCATTTTTCTTTCCGCTA | |
| | ATTTATAATGGTTTTGAAGTGGTTGTTCATTCTCAAA | |
| | CATAGACTTTTAAATGT | |
| 16 | AGAAATTAAGCTCTTTTAATGGATGTTTCTTGTACAT | IMPG1 Full |
| | GTCATTTTAGAAAACATCTGACCCTAACTGTCAGCCT | Promoter* |
| | TATTCTCTGTTTGGCAGAACTTCCCCTGGCTCTCTGT | |
| | GTCACTGTAACAGGTGAATAACTAAGAAAAAACTGT | |
| | GTCTGTAGACACTTGTTTATAATGGCATTCAGGGTCC | |
| | TGGAGCTAGGCTGACAGATGCTCCTCCAGAAGGTTA | |
| | ATGAGATAAAGGTTCCTCCAGCTGGCCCTTAAGCAG | |
| | AGATTACACCTGAGGGAAAGACAAGCAGATTATTCC | |
| | AGAAACAGACACTGCTACATGTTCTTCATAAATTAA | |
| | CACCCTCATAAAGGTAAACCAAGAAGGTTATC | |
| 17 | GTGGACCCTAACACACTGCAGGAGTGGTATTTGTAT | IMPG1 Full |
| | CCTGCTAACTCTAGCTGAAGTGCTAGCACCTCACCA | Downstream |
| | GTGCCAGAAAACCAGCACACAGCAGCTCAGGCCAG | Enhancer* |
| | GAGGAGAGGCTTGCACCATGCCACAGAGCTCTGCAG | |
| | CCCCTCACAGACTACCTGGAAGCTGGCTTGGTTCTGT | |
| | TTCCTTTCACACTTGCTGGCACTGAAACAGTTAGGAC | |
| | AAACCTATTGAGCGCTATTCTCTGATCATCCCTCAGT | |
| | AGTAAACAGCTCTAATGACAAGGAGAACAGCCCTGC | |
| | TTGAATAAACTTTTAAATGCTTTTCAAAATTAAGCCA | |
| | CTGTGACTATTTTAACAAAAATCTTAAATCTTAAAA | |
| | ATATATCACTCAGTTATCATGTGTGGAAAATTGAGA | |
| | ACGTTTTTTGAGAAAGGTTACTGTAAGACTTAAGGA | |
| | AGAAAGACCTAAATCCCCACTGTGATGGTATTTGGA | |
| | AGGTAATTAGGTCAAAGGTAAGAGCCTTTGGGATGT | |
| | AATTAGGTCATGAGGGCCGAGCCCTTATAAGAAGAG | |
| | ACAAAAGAGGGATGATTTCTCTCTCCCTTTCTCTCTC | |
| | TCTCTTTCTTTCTCTCTCTCTCCCTTTCTCTCCCTTTCT | |
| | CTCTCTCTTTCTCTCTCTCCTTTTCCGCCATGTGGGGA | |
| | TACAACTAGAGGACAGCTGCCTACAAACTAAGAAG | |
| | AGTGACCTCACTAGACACTGGTTTTTCTGGTGCCTTG | |
| | A | |
| 18 | GTGGACCCTAACACACTGCAGGAGTGGTATTTGTAT | IMPG1 Full |
| | CCTGCTAACTCTAGCTGAAGTGCTAGCACCTCACCA | Promoter* |
| | GTGCCAGAAAACCAGCACACAGCAGCTCAGGCCAG | IMPG1 Full |
| | GAGGAGAGGCTTGCACCATGCCACAGAGCTCTGCAG | Downstream |
| | CCCCTCACAGACTACCTGGAAGCTGGCTTGGTTCTGT | Enhancer* |
| | TTCCTTTCACACTTGCTGGCACTGAAACAGTTAGGAC | (IMPG1-PE1*) |
| | AAACCTATTGAGCGCTATTCTCTGATCATCCCTCAGT | in FIGs. 27-32 |
| | AGTAAACAGCTCTAATGACAAGGAGAACAGCCCTGC | |
| | TTGAATAAACTTTTAAATGCTTTTCAAAATTAAGCCA | |
| | CTGTGACTATTTTAACAAAAATCTTAAATCTTAAAA | |
| | ATATATCACTCAGTTATCATGTGTGGAAAATTGAGA | |
| | ACGTTTTTTGAGAAAGGTTACTGTAAGACTTAAGGA | |
| | AGAAAGACCTAAATCCCCACTGTGATGGTATTTGGA | |
| | AGGTAATTAGGTCAAAGGTAAGAGCCTTTGGGATGT | |
| | AATTAGGTCATGAGGGCCGAGCCCTTATAAGAAGAG | |
| | ACAAAAGAGGGATGATTTCTCTCTCCCTTTCTCTCTC | |
| | TCTCTTTCTTTCTCTCTCTCTCCCTTTCTCTCCCTTTCT | |
| | CTCTCTCTTTCTCTCTCTCCTTTTCCGCCATGTGGGGA | |
| | TACAACTAGAGGACAGCTGCCTACAAACTAAGAAG | |
| | AGTGACCTCACTAGACACTGGTTTTTCTGGTGCCTTG | |
| | AAGAAATTAAGCTCTTTTAATGGATGTTTCTTGTACA | |
| | TGTCATTTTAGAAAACATCTGACCCTAACTGTCAGCC | |
| | TTATTCTCTGTTTGGCAGAACTTCCCCTGGCTCTCTG | |
| | TGTCACTGTAACAGGTGAATAACTAAGAAAAAACTG | |
| | TGTCTGTAGACACTTGTTTATAATGGCATTCAGGGTC | |
| | CTGGAGCTAGGCTGACAGATGCTCCTCCAGAAGGTT | |
| | AATGAGATAAAGGTTCCTCCAGCTGGCCCTTAAGCA | |
| | GAGATTACACCTGAGGGAAAGACAAGCAATTATTCC | |
| | AGAAACAGACACTGCTACATGTTCTTCATAAATTAA | |
| | CACCCTCATAAAGGTAAACCAAGAAGGTTATC | |
| 19 | TCCTATTGTTAACTTTGAACTTCCAATTCATTTGGAG | IMPG1 Full |
| | AAGTGGTTTCCTCGTCATGTAATAAAGACCAAACGG | Promoter* And |
| | GAAGATATTCGAGAGCTCATCTTGAAACTTCATTTA | IMPG2 Full |
| | CAGCAACAGAAGGGCAGCAGTAGCTAGTTAATCTGT | Upstream |
| | ACAAACATGACACAGATGTTCTCTAAGATTACTGGA | Enhancer |
| | AAGCCTCTTACCAGCATTTGTGTTAGCCAGCTCACA | |
| | GAGAAGAAAATAACTTGCAGTAGTTTTATAATAAGT | |
| | CATTGGAACATTATTTAAAATATGTAGGACACATTA | |
| | TTAGAATTGTTGGGATCTCATAGATGGAATGGGAAT | |
| | GGGGGTGATATAGATAAACTTACTAGATATAAATTA | |
| | AAATTTTATAAATATTTCATATTTTTCTGAGTAAATA | |
| | TGATTGGATTATGCAACAGCATATGTAATATGGGAA | |
| | TGTTTTGTAGATAATAAAACTTACATGATCTGTACTT | |
| | CCACGTGACTGGGTGCTGAGGGGAGTTAAAGCCTCC | |
| | CTGGTGCCAGCCCCAGTGCTTGTCAAATTTGCTGAC | |
| | AGGTCACATCATATTGTAATTCTATTCTTTGCAGCTC | |
| | AAGCATGCAGTAAGAAATTAAGCTCTTTTAATGGAT | |
| | GTTTCTTGTACATGTCATTTTAGAAAACATCTGACCC | |
| | TAACTGTCAGCCTTATTCTCTGTTTGGCAGAACTTCC | |
| | CCTGGCTCTCTGTGTCACTGTAACAGGTGAATAACT | |
| | AAGAAAAAACTGTGTCTGTAGACACTTGTTTATAAT | |
| | GGCATTCAGGGTCCTGGAGCTAGGCTGACAGATGCT | |
| | CCTCCAGAAGGTTAATGAGATAAAGGTTCCTCCAGC | |
| | TGGCCCTTAAGCAGAGATTACACCTGAGGGAAAGAC | |
| | AAGCAGATTATTCCAGAAACAGACACTGCTACATGT | |
| | TCTTCATAAATTAACACCCTCATAAAGGTAAACCAA | |
| | GAAGGTTATC | |
| 20 | TCCTATTGTTAACTTTGAACTTCCAATTCATTTGGAG | IMPG1 Full |
| | AAGTGGTTTCCTCGTCATGTAATAAAGACCAAACGG | Promoter* And |
| | GAAGATATTCGAGAGCTCATCTTGAAACTTCATTTA | IMPG2 Short |
| | CAGCAACAGAAGGGCAGCAGTAGCTAGTTAATCTGT | Upstream |
| | ACAAACATGACACAGATGTTCTCTAAGATTACTGGA | Enhancer |
| | AAGCCTCTTACCAGCATTTGTGTTAGCCAGCTCACA | |
| | GAGAAGAAAATAACTTGCAGTAGTTTTATAATAAGT | |
| | CATTGGAACATTATTTAAAATATGTAGGACACATTA | |
| | TTAGAATTGTTAGAAATTAAGCTCTTTTAATGGATGT | |
| | TTCTTGTACATGTCATTTTAGAAAACATCTGACCCTA | |
| | ACTGTCAGCCTTATTCTCTGTTTGGCAGAACTTCCCC | |
| | TGGCTCTCTGTGTCACTGTAACAGGTGAATAACTAA | |
| | GAAAAAACTGTGTCTGTAGACACTTGTTTATAATGG | |
| | CATTCAGGGTCCTGGAGCTAGGCTGACAGATGCTCC | |
| | TCCAGAAGGTTAATGAGATAAAGGTTCCTCCAGCTG | |
| | GCCCTTAAGCAGAGATTACACCTGAGGGAAAGACA | |
| | AGCAGATTATTCCAGAAACAGACACTGCTACATGTT | |
| | CTTCATAAATTAACACCCTCATAAAGGTAAACCAAG | |
| | AAGGTTATC | |
| 21 | TAGTTATTAATAGTAATCAATTACGGGGTCATTAGTT | CMV Full |
| | CATAGCCCATATATGGAGTTCCGCGTTACATAACTT | Promoter |
| | ACGGTAAATGGCCCGCCTGGCTGACCGCCCAACGAC | |
| | CCCCGCCCATTGACGTCAATAATGACGTATGTTCCC | |
| | ATAGTAACGCCAATAGGGACTTTCCATTGACGTCAA | |
| | TGGGTGGAGTATTTACGGTAAACTGCCCACTTGGCA | |
| | GTACATCAAGTGTATCATATGCCAAGTACGCCCCCT | |
| | ATTGACGTCAATGACGGTAAATGGCCCGCCTGGCAT | |
| | TATGCCCAGTACATGACCTTATGGGACTTTCCTACTT | |
| | GGCAGTACATCTACGTATTAGTCATCGCTATTACCAT | |
| | GGTGATGCGGTTTTGGCAGTACATCAATGGGCGTGG | |
| | ATAGCGGTTTGACTCACGGGGATTTCCAAGTCTCCA | |
| | CCCCATTGACGTCAATGGGAGTTTGTTTTGGCACCA | |
| | AAATCAACGGGACTTTCCAAAATGTCGTAACAACTC | |
| | CGCCCCATTGACGCAAATGGGCGGTAGGCGTGTACG | |
| | GTGGGAGGTCTATATAAGCAGAGCTGGTTTAGTGAA | |
| | CCGTCAGATC | |
| 22 | TCCTATTGTTAACTTTGAACTTCCAATTCATTTGGAG | IMPG2-PE1 and |
| | AAGTGGTTTCCTCGTCATGTAATAAAGACCAAACGG | GFP sequence in |
| | GAAGATATTCGAGAGCTCATCTTGAAACTTCATTTA | AAV plasmid |
| | CAGCAACAGAAGGGCAGCAGTAGCTAGTTAATCTGT | |
| | ACAAACATGACACAGATGTTCTCTAAGATTACTGGA | |
| | AAGCCTCTTACCAGCATTTGTGTTAGCCAGCTCACA | |
| | GAGAAGAAAATAACTTGCAGTAGTTTTATAATAAGT | |
| | CATTGGAACATTATTTAAAATATGTAGGACACATTA | |
| | TTAGAATTGTTGGGATCTCATAGATGGAATGGGAAT | |
| | GGGGGTGATATAGATAAACTTACTAGATATAAATTA | |
| | AAATTTTATAAATATTTCATATTTTTCTGAGTAAATA | |
| | TGATTGGATTATGCAACAGCATATGTAATATGGGAA | |
| | TGTTTTGTAGATAATAAAACTTACATGATCTGTACTT | |
| | CCACGTGACTGGGTGCTGAGGGGAGTTAAAGCCTCC | |
| | CTGGTGCCAGCCCCAGTGCTTGTCAAATTTGCTGAC | |
| | AGGTCACATCATATTGTAATTCTATTCTTTGCAGCTC | |
| | AAGCATGCAGTAAGTTAATTAGTTAATTTAATCCTC | |
| | ACATTAGCTCTACCATGAGTTTACTATTTCTATTCCA | |
| | TTTTATACGTAAGGAAGGAGACAAAGTAAGTGATTT | |
| | TTCTATCAAGGAAGGAAATTTGCAAGAGAATAGTTT | |
| | CATTACAAAAACTAAATTTGTACGTAGCTCTGTATTA | |
| | TTGAAATAGGTAGATATAGTCAGTCTGGACTTTTTAT | |
| | GCTTATACATCTTAGTCCCTAGGAAAACCCAGAACT | |
| | AACAGATTCAGAAAAGTTGGAAAAATCAGTGAATTA | |
| | TATGTGAAACACATTATTCTTAGTGGACTGCTTGTTA | |
| | AAGGCAAGGAGAGTGTTAGTAAAGAGCTTAGGTAG | |
| | ATTAGAATAAAGAAATTGTCTCTCTCCATCTGCTCTA | |
| | ATTAGCTTATCTCACCAGCTTTTATAGCATGCTGGTT | |
| | ATTTCAGAAAAGAAGTGAGAGCTACTTTGAAAGGAC | |
| | AACCATTTTTCTTTCCGCTAATTTATAATGGTTTTGA | |
| | AGTGGTTGTTCATTCTCAAACATAGACTTTTAAATGT | |
| | TAGGTCTTTCCTATAACTCTTTGTTATTGGAAGTTTC | |
| | AAGGATTTGGACACTCAATTAAGGATTCTGTCCTCA | |
| | AGTTTGTACAAAAAAGCAGGCTGCCACCATGGTGAG | |
| | CAAGGGCGAGGAGCTGTTCACCGGGGTGGTGCCCAT | |
| | CCTGGTCGAGCTGGACGGCGACGTAAACGGCCACAA | |
| | GTTCAGCGTGTCCGGCGAGGGCGAGGGCGATGCCAC | |
| | CTACGGCAAGCTGACCCTGAAGTTCATCTGCACCAC | |
| | CGGCAAGCTGCCCGTGCCCTGGCCCACCCTCGTGAC | |
| | CACCCTGACCTACGGCGTGCAGTGCTTCAGCCGCTA | |
| | CCCCGACCACATGAAGCAGCACGACTTCTTCAAGTC | |
| | CGCCATGCCCGAAGGCTACGTCCAGGAGCGCACCAT | |
| | CTTCTTCAAGGACGACGGCAACTACAAGACCCGCGC | |
| | CGAGGTGAAGTTCGAGGGCGACACCCTGGTGAACCG | |
| | CATCGAGCTGAAGGGCATCGACTTCAAGGAGGACG | |
| | GCAACATCCTGGGGCACAAGCTGGAGTACAACTACA | |
| | ACAGCCACAACGTCTATATCATGGCCGACAAGCAGA | |
| | AGAACGGCATCAAGGTGAACTTCAAGATCCGCCACA | |
| | ACATCGAGGACGGCAGCGTGCAGCTCGCCGACCACT | |
| | ACCAGCAGAACACCCCCATCGGCGACGGCCCCGTGC | |
| | TGCTGCCCGACAACCACTACCTGAGCACCCAGTCCG | |
| | CCCTGAGCAAAGACCCCAACGAGAAGCGCGATCAC | |
| | ATGGTCCTGCTGGAGTTCGTGACCGCCGCCGGGATC | |
| | ACTCTCGGCATGGACGAGCTGTACAAGTAA | |
| 23 | AAAACGTGCTTTTTCCACGGAACTGTGCAACCCACA | IMPG2 Full |
| | GATCAGAAGATCTCACTTGGGAGCCCATACCTCCGG | Downstream |
| | GGCCTAGGGTCCCAACCACAGAACCAAGCAGATTTT | Enhancer (E2) |
| | CAACAGCCACTAAGCTAGAATCTGCTTAAGCCTGCG | |
| | GAGCTCCCCGCGGGAGGGGCAACCAGCACCACCGCT | |
| | GTGGCTGCCTGCTGTCTAAGCCCTTTGAACTCCTTTG | |
| | CAGGAGGGGCTACAGCCAACACTGGGACTGATAGCT | |
| | GCCTAACACACTAAGCTCCCAGGGTGGGGGAAGGGT | |
| | GGCAGCCATCTCTATAGCTCCAGGCCATGTCTTTCCC | |
| | CTGCTGGAGCCAGGGAGGCTGGACGGCTTGCTCCCA | |
| | AGAGGTATACCCCACAGTCCAACACACTAGCTGTGG | |
| | CAGACTGCGGACAGAGCGCCTCTTCAGGCCGGACCT | |
| | TGACATGTCCCTGCTCACTGGGTGGGGCCTCCTTGCA | |
| | GGAACTCCAACAACTCCATCCAGGGACTTAGGGACA | |
| | GACTCTAATCTTCCTGGGCTTGAGCCTCTAGAGGGA | |
| | GGGGTGGCCACAGTCTCTGCGGACGGGTAGATTTAG | |
| | TCTTCCCTCCTGGTAGTGCTGAGGAATCT | |
| 24 | AAAACGTGCTTTTTCCACGGAACTGTGCAACCCACA | IMPG2 Full |
| | GATCAGAAGATCTCACTTGGGAGCCCATACCTCCGG | Promoter |
| | GGCCTAGGGTCCCAACCACAGAACCAAGCAGATTTT | IMPG2 Full |
| | CAACAGCCACTAAGCTAGAATCTGCTTAAGCCTGCG | Downstream |
| | GAGCTCCCCGCGGGAGGGGCAACCAGCACCACCGCT | Enhancer |
| | GTGGCTGCCTGCTGTCTAAGCCCTTTGAACTCCTTTG | (IMPG2-PE2) |
| | CAGGAGGGGCTACAGCCAACACTGGGACTGATAGCT | |
| | GCCTAACACACTAAGCTCCCAGGGTGGGGGAAGGGT | |
| | GGCAGCCATCTCTATAGCTCCAGGCCATGTCTTTCCC | |
| | CTGCTGGAGCCAGGGAGGCTGGACGGCTTGCTCCCA | |
| | AGAGGTATACCCCACAGTCCAACACACTAGCTGTGG | |
| | CAGACTGCGGACAGAGCGCCTCTTCAGGCCGGACCT | |
| | TGACATGTCCCTGCTCACTGGGTGGGGCCTCCTTGCA | |
| | GGAACTCCAACAACTCCATCCAGGGACTTAGGGACA | |
| | GACTCTAATCTTCCTGGGCTTGAGCCTCTAGAGGGA | |
| | GGGGTGGCCACAGTCTCTGCGGACGGGTAGATTTAG | |
| | TCTTCCCTCCTGGTAGTGCTGAGGAATCTAGTTAATT | |
| | AGTTAATTTAATCCTCACATTAGCTCTACCATGAGTT | |
| | TACTATTTCTATTCCATTTTATACGTAAGGAAGGAGA | |
| | CAAAGTAAGTGATTTTTCTATCAAGGAAGGAAATTT | |
| | GCAAGAGAATAGTTTCATTACAAAAACTAAATTTGT | |
| | ACGTAGCTCTGTATTATTGAAATAGGTAGATATAGT | |
| | CAGTCTGGACTTTTTATGCTTATACATCTTAGTCCCT | |
| | AGGAAAACCCAGAACTAACAGATTCAGAAAAGTTG | |
| | GAAAAATCAGTGAATTATATGTGAAACACATTATTC | |
| | TTAGTGGACTGCTTGTTAAAGGCAAGGAGAGTGTTA | |
| | GTAAAGAGCTTAGGTAGATTAGAATAAAGAAATTGT | |
| | CTCTCTCCATCTGCTCTAATTAGCTTATCTCACCAGC | |
| | TTTTATAGCATGCTGGTTATTTCAGAAAAGAAGTGA | |
| | GAGCTACTTTGAAAGGACAACCATTTTTCTTTCCGCT | |
| | AATTTATAATGGTTTTGAAGTGGTTGTTCATTCTCAA | |
| | ACATAGACTTTTAAATGTTAGGTCTTTCCTATAACTC | |
| | TTTGTTATTGGAAGTTTCAAGGATTTGGACACTCAAT | |
| | TAAGGATTCTGTCCT | |
| 25 | GATTGAACCCCTCTGCTTACAACATCTGTTCCCTCCC | IMPG1 Full |
| | CAGGCTCCCTAGCAGGGCCCCAACCACCTCACAGCC | Downstream |
| | TGCCATCAGATAACTAATCAGCCCCACCACAGCCCA | Enhancer in |
| | ATGCCTGCATTAGCACATTCAGGCCTAATCAGGAAT | FIGs. 6-7 |
| | TAATGGACAGTGCTTCGCAGTAATTTAATTTCCACA | |
| | ATAAACCTAATGAGCAATTTAATACAAATGTCAAAA | |
| | GGAATCATTTAGTTGTTGTCTGAATGCTTTCTCCTGT | |
| | GCAAACCTTCATGAAAACTCAACACAGCTGCCTTTT | |
| | TATAGCCCAGTTAACATACATTCCAGCAGAATCAGG | |
| | GAGGCCCCTGTGCATTTAGTATTATTAATAAGGTTA | |
| | ACTGGGCACTTAATTAGCAATTTAGTGGTTAGTGCTC | |
| | CATGACTAGAAGGTCCCCAGAAGATTATTTAGCAGA | |
| | ATTAACTGAGGCTTTTGTCATATCCTCCCTAGATGCC | |
| | TTCCTCACACTCCTAAACAAAGCTGACTCTTAATCAA | |
| | TCCCAGATAGGTTCACAAAGAGGGCATGGACATAAA | |
| | CAATAAATCCCTTCCACCCAAGCCAATGCCATGGAG | |
| | CCCTCCTGGAGAGGAGGAGGGCTCTCCCCCGCAGTG | |
| | CTCCCTGTTGCTGATGCTGCAGCTGCAGGCATCTACA | |
| | ATGCCAGTCCCAGGGTCGATTGACTGTTTTCATGTAT | |
| | ATAACAGCATCTCTGGTATCCTGGAGAAGAAAAATG | |
| | GATGATTCAGGATCTGAATC | |
| 26 | ACAGAAATTAAGCTCTTTTAATGGATGTTTCTTGTAC | IMPG1 Full |
| | ATGTCATTTTAGAAAACATCTGACCCTAACTGTCAG | Promoter in |
| | CCTTATTCTCTGTTTGGCAGAACTTCCCCTGGCTCTC | FIGs. 6-7 |
| | TGTGTCACTGTAACAGGTGAATAACTAAGAAAAAAC | |
| | TGTGTCTGTAGACACTTGTTTATAATGGCATTCAGGG | |
| | TCCTGGAGCTAGGCTGACAGATGCTCCTCCAGAAGG | |
| | TTAATGAGATAAAGGTTCCTCCAGCTGGCCCTTAAG | |
| | CAGAGATTACACCTGAGGGAAAGACAAGCAGATTA | |
| | TTCCAGAAACAGACACTGCTACATGTTCTTCATAAA | |
| | TTAACACCCTCATAAAGGTAAACCAAGAAGGTTATC | |
| | CTCAATCATCTGGTATCAATATATAATTATTTTTCAC | |
| 27 | GATTGAACCCCTCTGCTTACAACATCTGTTCCCTCCC | IMPG1 Full |
| | CAGGCTCCCTAGCAGGGCCCCAACCACCTCACAGCC | Promoter Full |
| | TGCCATCAGATAACTAATCAGCCCCACCACAGCCCA | Downstream |
| | ATGCCTGCATTAGCACATTCAGGCCTAATCAGGAAT | Enhancer in |
| | TAATGGACAGTGCTTCGCAGTAATTTAATTTCCACA | FIGs.6-7 |
| | ATAAACCTAATGAGCAATTTAATACAAATGTCAAAA | (IMPG1-PE1) |
| | GGAATCATTTAGTTGTTGTCTGAATGCTTTCTCCTGT | |
| | GCAAACCTTCATGAAAACTCAACACAGCTGCCTTTT | |
| | TATAGCCCAGTTAACATACATTCCAGCAGAATCAGG | |
| | GAGGCCCCTGTGCATTTAGTATTATTAATAAGGTTA | |
| | ACTGGGCACTTAATTAGCAATTTAGTGGTTAGTGCTC | |
| | CATGACTAGAAGGTCCCCAGAAGATTATTTAGCAGA | |
| | ATTAACTGAGGCTTTTGTCATATCCTCCCTAGATGCC | |
| | TTCCTCACACTCCTAAACAAAGCTGACTCTTAATCAA | |
| | TCCCAGATAGGTTCACAAAGAGGGCATGGACATAAA | |
| | CAATAAATCCCTTCCACCCAAGCCAATGCCATGGAG | |
| | CCCTCCTGGAGAGGAGGAGGGCTCTCCCCCGCAGTG | |
| | CTCCCTGTTGCTGATGCTGCAGCTGCAGGCATCTACA | |
| | ATGCCAGTCCCAGGGTCGATTGACTGTTTTCATGTAT | |
| | ATAACAGCATCTCTGGTATCCTGGAGAAGAAAAATG | |
| | GATGATTCAGGATCTGAATCACAGAAATTAAGCTCT | |
| | TTTAATGGATGTTTCTTGTACATGTCATTTTAGAAAA | |
| | CATCTGACCCTAACTGTCAGCCTTATTCTCTGTTTGG | |
| | CAGAACTTCCCCTGGCTCTCTGTGTCACTGTAACAGG | |
| | TGAATAACTAAGAAAAAACTGTGTCTGTAGACACTT | |
| | GTTTATAATGGCATTCAGGGTCCTGGAGCTAGGCTG | |
| | ACAGATGCTCCTCCAGAAGGTTAATGAGATAAAGGT | |
| | TCCTCCAGCTGGCCCTTAAGCAGAGATTACACCTGA | |
| | GGGAAAGACAAGCAGATTATTCCAGAAACAGACAC | |
| | TGCTACATGTTCTTCATAAATTAACACCCTCATAAAG | |
| | GTAAACCAAGAAGGTTATCCTCAATCATCTGGTATC | |
| | AATATATAATTATTTTTCAC | |
| 28 | TTTGGCAAGTAAGTTTCTTATAAGCTTACCAGTATTT | IMPG2 |
| | TGCAAATACAACTATGCAAATATATTTAATGGTCAT | Palindromic |
| | TTAGGTTTATTAGCTTTTATAAAGGCTGAAAATGTGG | Enhancer |
| | TTTATTTGAGGCTGTATTGAAAAAATATACTTGAGCT | Sequence |
| | TTTCCTAAAGCATAAAATAACATTGAGGGTGATTTA | |
| | AGAGGAATGTGGTTTAGATCTTTACAATACACTTTTT | |
| | TTCAGAGAATTTTGCCAGAGATAACATGAAATAAAA | |
| | TATAATTTCATTGCTATTTGATAGTAAATCTTTACTT | |
| | GATTTAAAAAACTGTTCTAATATAGAGAATTTCATCT | |
| | GCAGGGAAAATGTTTTCTTGGTTAAGAGTTCCTTTAA | |
| | GCAACAAAGTTATCGCACAGCTATCATTTATATTAA | |
| | GTGCTTAATATGTTCTAAGCAAAGTGAAGATTGAAT | |
| | TAGTTAATTAGTTAATTTAATCCTCACATTAGCTCTA | |
| | CCATACTATTTCTATTCCATTTTATACGTAAGGAAGG | |
| | AGACAAAGGAAATTTGCAAGAGAATAGTTTCATTAC | |
| | AAAAACTAAATTTGTACGTAGCTCTGTATTATTGAA | |
| | ATAGGTAGATATAGTCAGTCTGGACTTTTTATGCTTA | |
| | TATGGAAAAATCAGTGAATTATATGTGAAACACATT | |
| | ATTCTTAGTTAGAATAAAGAAATTGTCTCTCTCCATC | |
| | TGCTCTAATTAGCTTATCTCACCAGCTTT | |
| 29 | TTTGGCAAGTAAGTTTCTTATAAGCTTACCAGTATTT | CMV promoter |
| | TGCAAATACAACTATGCAAATATATTTAATGGTCAT | and IMPG2 |
| | TTAGGTTTATTAGCTTTTATAAAGGCTGAAAATGTGG | palindromic |
| | TTTATTTGAGGCTGTATTGAAAAAATATACTTGAGCT | sequence |
| | TTTCCTAAAGCATAAAATAACATTGAGGGTGATTTA | |
| | AGAGGAATGTGGTTTAGATCTTTACAATACACTTTTT | |
| | TTCAGAGAATTTTGCCAGAGATAACATGAAATAAAA | |
| | TATAATTTCATTGCTATTTGATAGTAAATCTTTACTT | |
| | GATTTAAAAAACTGTTCTAATATAGAGAATTTCATCT | |
| | GCAGGGAAAATGTTTTCTTGGTTAAGAGTTCCTTTAA | |
| | GCAACAAAGTTATCGCACAGCTATCATTTATATTAA | |
| | GTGCTTAATATGTTCTAAGCAAAGTGAAGATTGAAT | |
| | TAGTTAATTAGTTAATTTAATCCTCACATTAGCTCTA | |
| | CCATACTATTTCTATTCCATTTTATACGTAAGGAAGG | |
| | AGACAAAGGAAATTTGCAAGAGAATAGTTTCATTAC | |
| | AAAAACTAAATTTGTACGTAGCTCTGTATTATTGAA | |
| | ATAGGTAGATATAGTCAGTCTGGACTTTTTATGCTTA | |
| | TATGGAAAAATCAGTGAATTATATGTGAAACACATT | |
| | ATTCTTAGTTAGAATAAAGAAATTGTCTCTCTCCATC | |
| | TGCTCTAATTAGCTTATCTCACCAGCTTT | |
| | TAGGCGTGTACGGTGGGAGGTCTATATAAGCAGAGC | |
| | T CGTTTAGTGAACCGCCACCATG | |

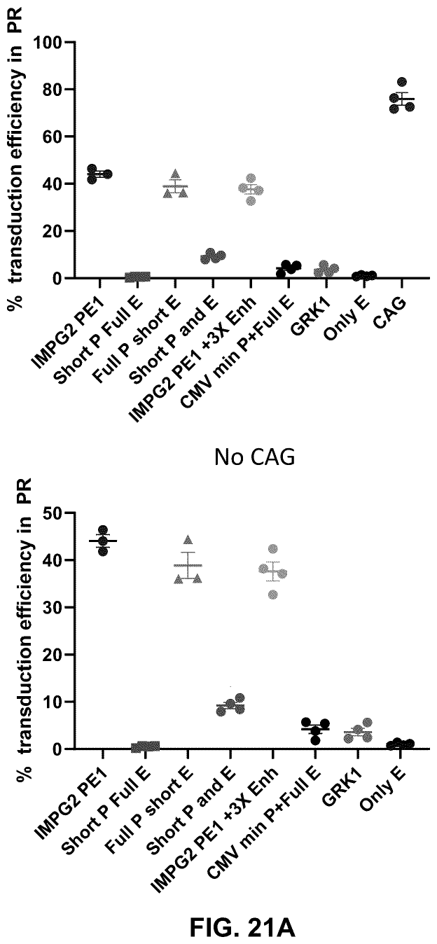

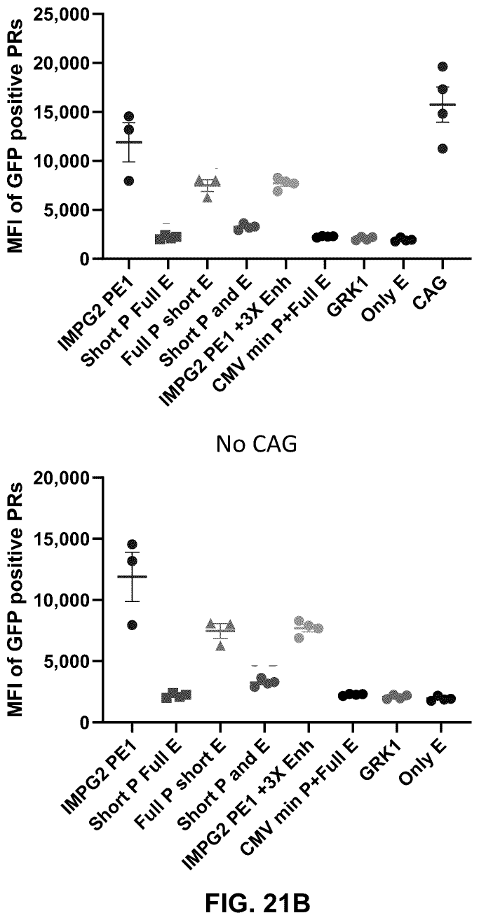

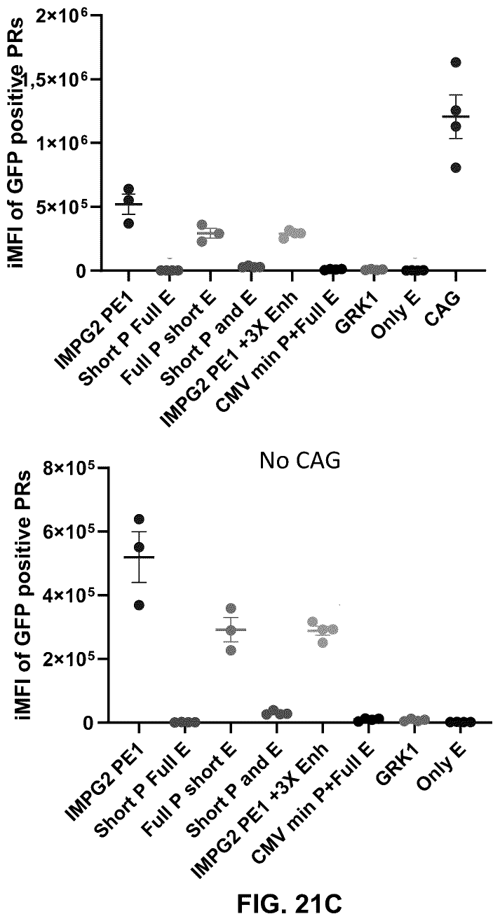

TABLE 1
Statistical Results in FIG. 21A.

| | | Adjusted |
|---|---|---|
| Tukey's multiple comparisons test | Summary | P Value |
| IMPG2 PE1 vs. Short P Full E | **** | <0.0001 |
| IMPG2 PE1 vs. Full P short E | ns | 0.431 |
| IMPG2 PE1 vs. Short P and E | **** | <0.0001 |
| IMPG2 PE1 vs. IMPG2 PE1 + 3X Enh | ns | 0.1277 |
| IMPG2 PE1 vs. CMV min P + Full E | **** | <0.0001 |
| IMPG2 PE1 vs. GRK1 | **** | <0.0001 |
| IMPG2 PE1 vs. Only E | **** | <0.0001 |
| IMPG2 PE1 vs. CAG | **** | <0.0001 |
| Short P Full E vs. Full P short E | **** | <0.0001 |
| Short P Full E vs. Short P and E | ** | 0.0065 |
| Short P Full E vs. IMPG2 PE1 + 3X Enh | **** | <0.0001 |
| Short P Full E vs. CMV min P + Full E | ns | 0.6734 |
| Short P Full E vs. GRK1 | ns | 0.8361 |
| Short P Full E vs. Only E | ns | >0.9999 |
| Short P Full E vs. CAG | **** | <0.0001 |
| Full P short E vs. Short P and E | **** | <0.0001 |
| Full P short E vs. IMPG2 PE1 + 3X Enh | ns | 0.9996 |
| Full P short E vs. CMV min P + Full E | **** | <0.0001 |
| Full P short E vs. GRK1 | **** | <0.0001 |
| Full P short E vs. Only E | **** | <0.0001 |
| Full P short E vs. CAG | **** | <0.0001 |
| Short P and E vs. IMPG2 PE1 + 3X Enh | **** | <0.0001 |
| Short P and E vs. CMV min P + Full E | ns | 0.3015 |
| Short P and E vs. GRK1 | ns | 0.1826 |
| Short P and E vs. Only E | * | 0.0121 |
| Short P and E vs. CAG | **** | <0.0001 |
| IMPG2 PE1 + 3X Enh vs. CMV min P + Full E | **** | <0.0001 |
| IMPG2 PE1 + 3X Enh vs. GRK1 | **** | <0.0001 |
| IMPG2 PE1 + 3X Enh vs. Only E | **** | <0.0001 |
| IMPG2 PE1 + 3X Enh vs. CAG | **** | <0.0001 |
| CMV min P + Full E vs. GRK1 | ns | >0.9999 |
| CMV min P + Full E vs. Only E | ns | 0.8183 |
| CMV min P + Full E vs. CAG | **** | <0.0001 |
| GRK1 vs. Only E | ns | 0.9332 |
| GRK1 vs. CAG | **** | <0.0001 |
| Only E vs. CAG | **** | <0.0001 |

ns: non-significant. P: promoter. E: enhancer.

TABLE 2
Statistical Results in FIG. 21B.

| | | Adjusted |
|---|---|---|
| Tukey's multiple comparisons test | Summary | P Value |
| IMPG2 PE1 vs. Short P Full E | **** | <0.0001 |
| IMPG2 PE1 vs. Full P short E | ns | 0.0579 |
| IMPG2 PE1 vs. Short P and E | **** | <0.0001 |
| IMPG2 PE1 vs. IMPG2 PE1 + 3X Enh | ns | 0.0525 |
| IMPG2 PE1 vs. CMV min P + Full E | **** | <0.0001 |
| IMPG2 PE1 vs. GRK1 | **** | <0.0001 |
| IMPG2 PE1 vs. Only E | **** | <0.0001 |
| IMPG2 PE1 vs. CAG | ns | 0.0927 |
| Short P Full E vs. Full P short E | ** | 0.0069 |
| Short P Full E vs. Short P and E | ns | 0.9896 |
| Short P Full E vs. IMPG2 PE1 + 3X Enh | ** | 0.0018 |
| Short P Full E vs. CMV min P + Full E | ns | >0.9999 |
| Short P Full E vs. GRK1 | ns | >0.9999 |
| Short P Full E vs. Only E | ns | >0.9999 |
| Short P Full E vs. CAG | **** | <0.0001 |
| Full P short E vs. Short P and E | ns | 0.05 |
| Full P short E vs. IMPG2 PE1 + 3X Enh | ns | >0.9999 |
| Full P short E vs. CMV min P + Full E | ** | 0.0082 |
| Full P short E vs. GRK1 | ** | 0.0056 |
| Full P short E vs. Only E | ** | 0.0043 |
| Full P short E vs. CAG | **** | <0.0001 |
| Short P and E vs. IMPG2 PE1 + 3X Enh | * | 0.0172 |
| Short P and E vs. CMV min P + Full E | ns | 0.994 |
| Short P and E vs. GRK1 | ns | 0.9812 |
| Short P and E vs. Only E | ns | 0.9635 |
| Short P and E vs. CAG | **** | <0.0001 |
| IMPG2 PE1 + 3X Enh vs. CMV min P + Full E | ** | 0.0022 |
| IMPG2 PE1 + 3X Enh vs. GRK1 | ** | 0.0015 |
| IMPG2 PE1 + 3X Enh vs. Only E | ** | 0.0011 |
| IMPG2 PE1 + 3X Enh vs. CAG | **** | <0.0001 |
| CMV min P + Full E vs. GRK1 | ns | >0.9999 |
| CMV min P + Full E vs. Only E | ns | >0.9999 |
| CMV min P + Full E vs. CAG | **** | <0.0001 |
| GRK1 vs. Only E | ns | >0.9999 |
| GRK1 vs. CAG | **** | <0.0001 |
| Only E vs. CAG | **** | <0.0001 |

ns: non-significant. P: promoter. E: enhancer.

TABLE 3
Statistical Results in FIG. 21C.

| | | Adjusted |
|---|---|---|
| Tukey's multiple comparisons test | Summary | P Value |
| IMPG2 PE1 vs. Short P Full E | *** | 0.0004 |
| IMPG2 PE1 vs. Full P short E | ns | 0.4281 |
| IMPG2 PE1 vs. Short P and E | *** | 0.0009 |
| IMPG2 PE1 vs. IMPG2 PE1 + 3X Enh | ns | 0.3255 |
| IMPG2 PE1 vs. CMV min P + Full E | *** | 0.0005 |
| IMPG2 PE1 vs. GRK1 | *** | 0.0005 |
| IMPG2 PE1 vs. Only E | *** | 0.0004 |
| IMPG2 PE1 vs. CAG | **** | <0.0001 |
| Short P Full E vs. Full P short E | ns | 0.108 |
| Short P Full E vs. Short P and E | ns | >0.9999 |
| Short P Full E vs. IMPG2 PE1 + 3X Enh | ns | 0.0713 |
| Short P Full E vs. CMV min P + Full E | ns | >0.9999 |
| Short P Full E vs. GRK1 | ns | >0.9999 |
| Short P Full E vs. Only E | ns | >0.9999 |
| Short P Full E vs. CAG | **** | <0.0001 |
| Full P short E vs. Short P and E | ns | 0.1909 |
| Full P short E vs. IMPG2 PE1 + 3X Enh | ns | >0.9999 |
| Full P short E vs. CMV min P + Full E | ns | 0.1286 |
| Full P short E vs. GRK1 | ns | 0.1239 |
| Full P short E vs. Only E | ns | 0.1101 |
| Full P short E vs. CAG | **** | <0.0001 |
| Short P and E vs. IMPG2 PE1 + 3X Enh | ns | 0.1368 |
| Short P and E vs. CMV min P + Full E | ns | >0.9999 |
| Short P and E vs. GRK1 | ns | >0.9999 |
| Short P and E vs. Only E | ns | >0.9999 |
| Short P and E vs. CAG | **** | <0.0001 |
| IMPG2 PE1 + 3X Enh vs. CMV min P + Full E | ns | 0.087 |
| IMPG2 PE1 + 3X Enh vs. GRK1 | ns | 0.0833 |
| IMPG2 PE1 + 3X Enh vs. Only E | ns | 0.0728 |
| IMPG2 PE1 + 3X Enh vs. CAG | **** | <0.0001 |
| CMV min P + Full E vs. GRK1 | ns | >0.9999 |
| CMV min P + Full E vs. Only E | ns | >0.9999 |
| CMV min P + Full E vs. CAG | **** | <0.0001 |
| GRK1 vs. Only E | ns | >0.9999 |
| GRK1 vs. CAG | **** | <0.0001 |
| Only E vs. CAG | **** | <0.0001 |

ns: non-significant. P: promoter. E: enhancer.

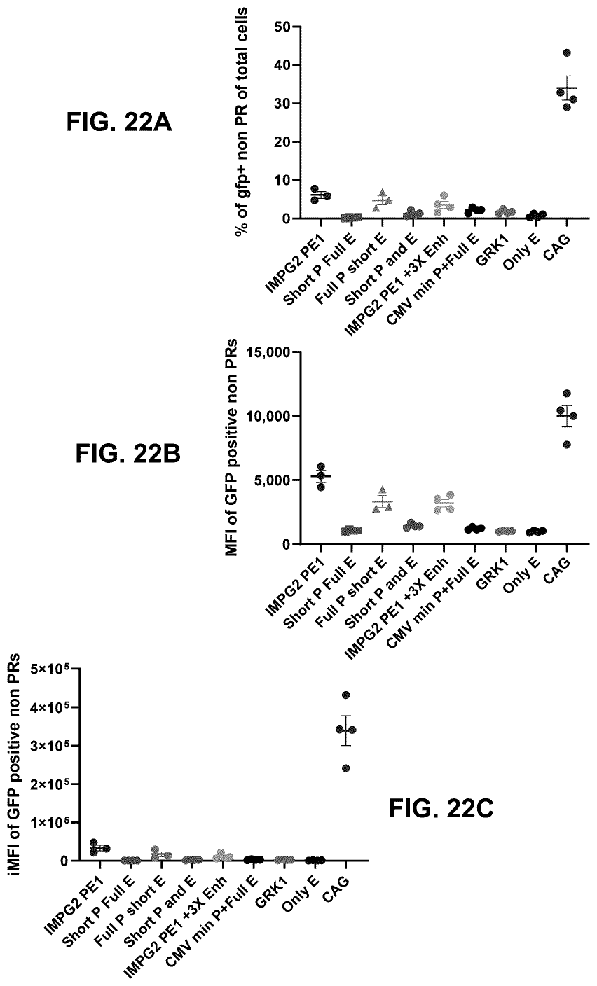

TABLE 4
Statistical Results in FIG. 22A.

| | | Adjusted |
|---|---|---|
| Tukey's multiple comparisons test | Summary | P Value |
| IMPG2 PE1 vs. Short P Full E | ns | 0.082 |
| IMPG2 PE1 vs. Full P short E | ns | 0.9987 |
| IMPG2 PE1 vs. Short P and E | ns | 0.2233 |
| IMPG2 PE1 vs. IMPG2 PE1 + 3X Enh | ns | 0.8932 |
| IMPG2 PE1 vs. CMV min P + Full E | ns | 0.481 |
| IMPG2 PE1 vs. GRK1 | ns | 0.3385 |
| IMPG2 PE1 vs. Only E | ns | 0.1425 |
| IMPG2 PE1 vs. CAG | **** | <0.0001 |
| Short P Full E vs. Full P short E | ns | 0.3124 |
| Short P Full E vs. Short P and E | ns | 0.9996 |
| Short P Full E vs. IMPG2 PE1 + 3X Enh | ns | 0.6128 |
| Short P Full E vs. CMV min P + Full E | ns | 0.9665 |
| Short P Full E vs. GRK1 | ns | 0.9942 |
| Short P Full E vs. Only E | ns | >0.9999 |
| Short P Full E vs. CAG | **** | <0.0001 |
| Full P short E vs. Short P and E | ns | 0.6165 |
| Full P short E vs. IMPG2 PE1 + 3X Enh | ns | 0.9989 |
| Full P short E vs. CMV min P + Full E | ns | 0.8886 |
| Full P short E vs. GRK1 | ns | 0.7693 |
| Full P short E vs. Only E | ns | 0.464 |
| Full P short E vs. CAG | **** | <0.0001 |
| Short P and E vs. IMPG2 PE1 + 3X Enh | ns | 0.9069 |
| Short P and E vs. CMV min P + Full E | ns | 0.9997 |
| Short P and E vs. GRK1 | ns | >0.9999 |
| Short P and E vs. Only E | ns | >0.9999 |
| Short P and E vs. CAG | **** | <0.0001 |
| IMPG2 PE1 + 3X Enh vs. CMV min P + Full E | ns | 0.9959 |
| IMPG2 PE1 + 3X Enh vs. GRK1 | ns | 0.9734 |
| IMPG2 PE1 + 3X Enh vs. Only E | ns | 0.7894 |
| IMPG2 PE1 + 3X Enh vs. CAG | **** | <0.0001 |
| CMV min P + Full E vs. GRK1 | ns | >0.9999 |
| CMV min P + Full E vs. Only E | ns | 0.9953 |
| CMV min P + Full E vs. CAG | **** | <0.0001 |
| GRK1 vs. Only E | ns | 0.9997 |
| GRK1 vs. CAG | **** | <0.0001 |
| Only E vs. CAG | **** | <0.0001 |

ns: non-significant. P: promoter. E: enhancer.

TABLE 5
Statistical Results in FIG. 22B.

| | | Adjusted |
|---|---|---|
| Tukey's multiple comparisons test | Summary | P Value |
| IMPG2 PE1 vs. Short P Full E | **** | <0.0001 |
| IMPG2 PE1 vs. Full P short E | * | 0.0431 |
| IMPG2 PE1 vs. Short P and E | **** | <0.0001 |
| IMPG2 PE1 vs. IMPG2 PE1 + 3X Enh | * | 0.0148 |
| IMPG2 PE1 vs. CMV min P + Full E | **** | <0.0001 |
| IMPG2 PE1 vs. GRK1 | **** | <0.0001 |
| IMPG2 PE1 vs. Only E | **** | <0.0001 |
| IMPG2 PE1 vs. CAG | **** | <0.0001 |
| Short P Full E vs. Full P short E | ** | 0.0071 |
| Short P Full E vs. Short P and E | ns | 0.9973 |
| Short P Full E vs. IMPG2 PE1 + 3X Enh | ** | 0.0057 |
| Short P Full E vs. CMV min P + Full E | ns | >0.9999 |
| Short P Full E vs. GRK1 | ns | >0.9999 |
| Short P Full E vs. Only E | ns | >0.9999 |
| Short P Full E vs. CAG | **** | <0.0001 |
| Full P short E vs. Short P and E | * | 0.0361 |
| Full P short E vs. IMPG2 PE1 + 3X Enh | ns | >0.9999 |
| Full P short E vs. CMV min P + Full E | * | 0.0141 |
| Full P short E vs. GRK1 | ** | 0.0054 |
| Full P short E vs. Only E | ** | 0.0049 |
| Full P short E vs. CAG | **** | <0.0001 |
| Short P and E vs. IMPG2 PE1 + 3X Enh | * | 0.0333 |
| Short P and E vs. CMV min P + Full E | ns | >0.9999 |
| Short P and E vs. GRK1 | ns | 0.9928 |
| Short P and E vs. Only E | ns | 0.9901 |
| Short P and E vs. CAG | **** | <0.0001 |
| IMPG2 PE1 + 3X Enh vs. CMV min P + Full E | * | 0.012 |
| IMPG2 PE1 + 3X Enh vs. GRK1 | ** | 0.0042 |
| IMPG2 PE1 + 3X Enh vs. Only E | ** | 0.0038 |
| IMPG2 PE1 + 3X Enh vs. CAG | **** | <0.0001 |
| CMV min P + Full E vs. GRK1 | ns | >0.9999 |
| CMV min P + Full E vs. Only E | ns | >0.9999 |
| CMV min P + Full E vs. CAG | **** | <0.0001 |
| GRK1 vs. Only E | ns | >0.9999 |
| GRK1 vs. CAG | **** | <0.0001 |
| Only E vs. CAG | **** | <0.0001 |

ns: non-significant. P: promoter. E: enhancer.

TABLE 6
Statistical Results in FIG. 22C.

| | | Adjusted |
|---|---|---|
| Tukey's multiple comparisons test | Summary | P Value |
| IMPG2 PE1 vs. Short P Full E | ns | 0.8151 |
| IMPG2 PE1 vs. Full P short E | ns | 0.9979 |
| IMPG2 PE1 vs. Short P and E | ns | 0.8475 |
| IMPG2 PE1 vs. IMPG2 PE1 + 3X Enh | ns | 0.9775 |
| IMPG2 PE1 vs. CMV min P + Full E | ns | 0.8676 |
| IMPG2 PE1 vs. GRK1 | ns | 0.8477 |
| IMPG2 PE1 vs. Only E | ns | 0.827 |
| IMPG2 PE1 vs. CAG | **** | <0.0001 |
| Short P Full E vs. Full P short E | ns | 0.9963 |
| Short P Full E vs. Short P and E | ns | >0.9999 |
| Short P Full E vs. IMPG2 PE1 + 3X Enh | ns | 0.9997 |
| Short P Full E vs. CMV min P + Full E | ns | >0.9999 |
| Short P Full E vs. GRK1 | ns | >0.9999 |
| Short P Full E vs. Only E | ns | >0.9999 |
| Short P Full E vs. CAG | **** | <0.0001 |
| Full P short E vs. Short P and E | ns | 0.998 |
| Full P short E vs. IMPG2 PE1 + 3X Enh | ns | >0.9999 |
| Full P short E vs. CMV min P + Full E | ns | 0.9987 |
| Full P short E vs. GRK1 | ns | 0.998 |
| Full P short E vs. Only E | ns | 0.997 |
| Full P short E vs. CAG | **** | <0.0001 |
| Short P and E vs. IMPG2 PE1 + 3X Enh | ns | 0.9999 |
| Short P and E vs. CMV min P + Full E | ns | >0.9999 |
| Short P and E vs. GRK1 | ns | >0.9999 |
| Short P and E vs. Only E | ns | >0.9999 |
| Short P and E vs. CAG | **** | <0.0001 |
| IMPG2 PE1 + 3X Enh vs. CMV min P + Full E | ns | >0.9999 |
| IMPG2 PE1 + 3X Enh vs. GRK1 | ns | 0.9999 |
| IMPG2 PE1 + 3X Enh vs. Only E | ns | 0.9998 |
| IMPG2 PE1 + 3X Enh vs. CAG | **** | <0.0001 |
| CMV min P + Full E vs. GRK1 | ns | >0.9999 |
| CMV min P + Full E vs. Only E | ns | >0.9999 |
| CMV min P + Full E vs. CAG | **** | <0.0001 |
| GRK1 vs. Only E | ns | >0.9999 |
| GRK1 vs. CAG | **** | <0.0001 |
| Only E vs. CAG | **** | <0.0001 |

ns: non-significant. P: promoter. E: enhancer.

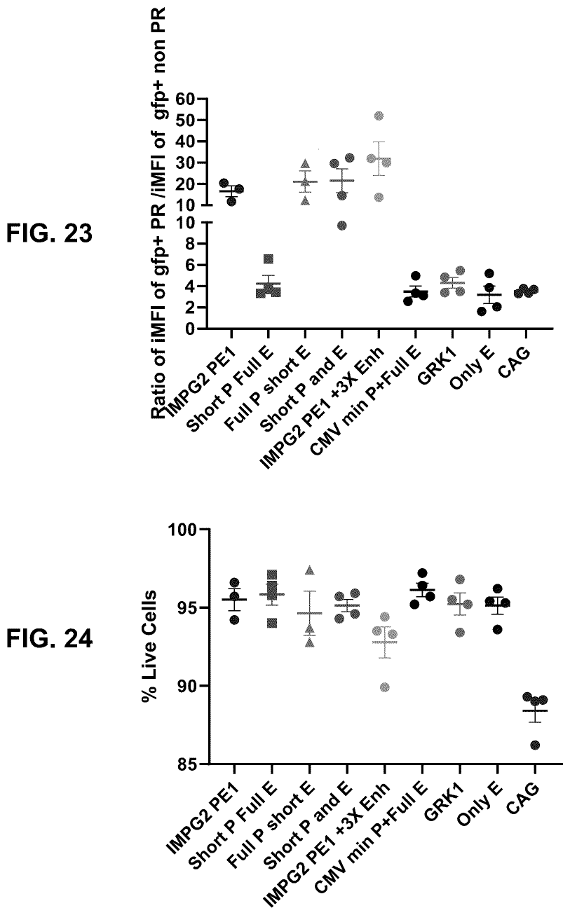

TABLE 7
Statistical Results in FIG. 23.

| | | Adjusted |
|---|---|---|
| Tukey's multiple comparisons test | Summary | P Value |
| IMPG2 PE1 vs. Short P Full E | ns | 0.4253 |
| IMPG2 PE1 vs. Full P short E | ns | 0.9971 |
| IMPG2 PE1 vs. Short P and E | ns | 0.9916 |
| IMPG2 PE1 vs. IMPG2 PE1 + 3X Enh | ns | 0.1762 |
| IMPG2 PE1 vs. CMV min P + Full E | ns | 0.3525 |
| IMPG2 PE1 vs. GRK1 | ns | 0.4322 |
| IMPG2 PE1 vs. Only E | ns | 0.3228 |
| IMPG2 PE1 vs. CAG | ns | 0.3541 |
| Short P Full E vs. Full P short E | ns | 0.1059 |
| Short P Full E vs. Short P and E | ns | 0.0536 |
| Short P Full E vs. IMPG2 PE1 + 3X Enh | *** | 0.0004 |
| Short P Full E vs. CMV min P + Full E | ns | >0.9999 |
| Short P Full E vs. GRK1 | ns | >0.9999 |
| Short P Full E vs. Only E | ns | >0.9999 |
| Short P Full E vs. CAG | ns | >0.9999 |
| Full P short E vs. Short P and E | ns | >0.9999 |
| Full P short E vs. IMPG2 PE1 + 3X Enh | ns | 0.587 |
| Full P short E vs. CMV min P + Full E | ns | 0.0809 |
| Full P short E vs. GRK1 | ns | 0.1085 |
| Full P short E vs. Only E | ns | 0.0716 |
| Full P short E vs. CAG | ns | 0.0814 |
| Short P and E vs. IMPG2 PE1 + 3X Enh | ns | 0.5411 |
| Short P and E vs. CMV min P + Full E | * | 0.0392 |
| Short P and E vs. GRK1 | ns | 0.0551 |
| Short P and E vs. Only E | * | 0.034 |
| Short P and E vs. CAG | * | 0.0395 |
| IMPG2 PE1 + 3X Enh vs. CMV min P + Full E | *** | 0.0003 |
| IMPG2 PE1 + 3X Enh vs. GRK1 | *** | 0.0004 |
| IMPG2 PE1 + 3X Enh vs. Only E | *** | 0.0002 |
| IMPG2 PE1 + 3X Enh vs. CAG | *** | 0.0003 |
| CMV min P + Full E vs. GRK1 | ns | >0.9999 |
| CMV min P + Full E vs. Only E | ns | >0.9999 |
| CMV min P + Full E vs. CAG | ns | >0.9999 |
| GRK1 vs. Only E | ns | >0.9999 |
| GRK1 vs. CAG | ns | >0.9999 |
| Only E vs. CAG | ns | >0.9999 |

ns: non-significant. P: promoter. E: enhancer.

TABLE 8
Statistical Results in FIG. 24.

| | | Adjusted |
|---|---|---|
| Tukey's multiple comparisons test | Summary | P Value |
| IMPG2 PE1 vs. Short P Full E | ns | >0.9999 |
| IMPG2 PE1 vs. Full P short E | ns | 0.9976 |
| IMPG2 PE1 vs. Short P and E | ns | >0.9999 |
| IMPG2 PE1 vs. IMPG2 PE1 + 3X Enh | ns | 0.2901 |
| IMPG2 PE1 vs. CMV min P + Full E | ns | 0.9996 |
| IMPG2 PE1 vs. GRK1 | ns | >0.9999 |
| IMPG2 PE1 vs. Only E | ns | >0.9999 |
| IMPG2 PE1 vs. CAG | **** | <0.0001 |
| Short P Full E vs. Full P short E | ns | 0.9717 |
| Short P Full E vs. Short P and E | ns | 0.9985 |
| Short P Full E vs. IMPG2 PE1 + 3X Enh | ns | 0.114 |
| Short P Full E vs. CMV min P + Full E | ns | >0.9999 |
| Short P Full E vs. GRK1 | ns | 0.9995 |
| Short P Full E vs. Only E | ns | 0.9985 |
| Short P Full E vs. CAG | **** | <0.0001 |
| Full P short E vs. Short P and E | ns | >0.9999 |
| Full P short E vs. IMPG2 PE1 + 3X Enh | ns | 0.748 |
| Full P short E vs. CMV min P + Full E | ns | 0.904 |
| Full P short E vs. GRK1 | ns | 0.9998 |
| Full P short E vs. Only E | ns | >0.9999 |
| Full P short E vs. CAG | *** | 0.0002 |
| Short P and E vs. IMPG2 PE1 + 3X Enh | ns | 0.3758 |
| Short P and E vs. CMV min P + Full E | ns | 0.9843 |
| Short P and E vs. GRK1 | ns | >0.9999 |
| Short P and E vs. Only E | ns | >0.9999 |
| Short P and E vs. CAG | **** | <0.0001 |
| IMPG2 PE1 + 3X Enh vs. CMV min P + Full E | ns | 0.0621 |
| IMPG2 PE1 + 3X Enh vs. GRK1 | ns | 0.3246 |
| IMPG2 PE1 + 3X Enh vs. Only E | ns | 0.3758 |
| IMPG2 PE1 + 3X Enh vs. CAG | ** | 0.0061 |
| CMV min P + Full E vs. GRK1 | ns | 0.992 |
| CMV min P + Full E vs. Only E | ns | 0.9843 |
| CMV min P + Full E vs. CAG | **** | <0.0001 |
| GRK1 vs. Only E | ns | >0.9999 |
| GRK1 vs. CAG | **** | <0.0001 |
| Only E vs. CAG | **** | <0.0001 |

ns: non-significant. P: promoter. E: enhancer.

TABLE 9
Statistical Results in FIG. 30A.

| | | Adjusted |
|---|---|---|
| Tukey's multiple comparisons test | Summary | P Value |
| IMPG2 PE1 vs. IMPG2 Prom only | **** | <0.0001 |
| IMPG2 PE1 vs. IMPG2 Prom center short Enh | **** | <0.0001 |
| IMPG2 PE1 vs. IMPG1 PE1 | ns | 0.5076 |
| IMPG2 PE1 vs. CAG | **** | <0.0001 |
| IMPG2 Prom only vs. IMPG2 Prom center short | ** | 0.003 |
| Enh | | |
| IMPG2 Prom only vs. IMPG1 PE1 | **** | <0.0001 |
| IMPG2 Prom only vs. CAG | **** | <0.0001 |
| IMPG2 Prom center short Enh vs. IMPG1 PE1 | **** | <0.0001 |
| IMPG2 Prom center short Enh vs. CAG | **** | <0.0001 |
| IMPG1 PE1 vs. CAG | **** | <0.0001 |

ns: non-significant. P: promoter. E: enhancer.

TABLE 10
Statistical Results in FIG. 30B.

| | | Adjusted |
|---|---|---|
| Tukey's multiple comparisons test | Summary | P Value |
| IMPG2 PE1 vs. IMPG2 Prom only | ns | 0.7891 |
| IMPG2 PE1 vs. IMPG2 Prom center short Enh | ns | 0.8269 |
| IMPG2 PE1 vs. IMPG1 PE1 | ns | >0.9999 |
| IMPG2 PE1 vs. CAG | *** | 0.0002 |
| IMPG2 Prom only vs. IMPG2 Prom center short | ns | >0.9999 |
| Enh | | |
| IMPG2 Prom only vs. IMPG1 PE1 | ns | 0.8354 |
| IMPG2 Prom only vs. CAG | **** | <0.0001 |
| IMPG2 Prom center short Enh vs. IMPG1 PE1 | ns | 0.869 |
| IMPG2 Prom center short Enh vs. CAG | **** | <0.0001 |
| IMPG1 PE1 vs. CAG | *** | 0.0002 |

ns: non-significant. P: promoter. E: enhancer.

TABLE 11
Statistical Results in FIG. 30C.

| | | Adjusted |
|---|---|---|
| Tukey's multiple comparisons test | Summary | P Value |
| IMPG2 PE1 vs. IMPG2 Prom only | ns | 0.942 |
| IMPG2 PE1 vs. IMPG2 Prom center short Enh | ns | 0.9517 |
| IMPG2 PE1 vs. IMPG1 PE1 | ns | >0.9999 |
| IMPG2 PE1 vs. CAG | *** | 0.0002 |
| IMPG2 Prom only vs. IMPG2 Prom center short | ns | >0.9999 |
| Enh | | |
| IMPG2 Prom only vs. IMPG1 PE1 | ns | 0.9629 |
| IMPG2 Prom only vs. CAG | **** | <0.0001 |
| IMPG2 Prom center short Enh vs. IMPG1 PE1 | ns | 0.9702 |
| IMPG2 Prom center short Enh vs. CAG | **** | <0.0001 |
| IMPG1 PE1 vs. CAG | *** | 0.0002 |

ns: non-significant. P: promoter. E: enhancer.

TABLE 12
Statistical Results in FIG. 31A.

| | | Adjusted |
|---|---|---|
| Tukey's multiple comparisons test | Summary | P Value |
| IMPG2 PE1 vs. IMPG2 Prom only | ns | 0.4342 |
| IMPG2 PE1 vs. IMPG2 Prom center short Enh | ns | 0.5655 |
| IMPG2 PE1 vs. IMPG1 PE1 | ns | 0.9962 |
| IMPG2 PE1 vs. CAG | **** | <0.0001 |
| IMPG2 Prom only vs. IMPG2 Prom center short | ns | 0.9993 |
| Enh | | |
| IMPG2 Prom only vs. IMPG1 PE1 | ns | 0.2666 |
| IMPG2 Prom only vs. CAG | **** | <0.0001 |
| IMPG2 Prom center short Enh vs. IMPG1 PE1 | ns | 0.3699 |
| IMPG2 Prom center short Enh vs. CAG | **** | <0.0001 |
| IMPG1 PE1 vs. CAG | **** | <0.0001 |

ns: non-significant. P: promoter. E: enhancer.

TABLE 13
Statistical Results in FIG. 31B.

| | | Adjusted |
|---|---|---|
| Tukey's multiple comparisons test | Summary | P Value |
| IMPG2 PE1 vs. IMPG2 Prom only | ns | 0.7781 |
| IMPG2 PE1 vs. IMPG2 Prom center short Enh | ns | 0.8034 |
| IMPG2 PE1 vs. IMPG1 PE1 | ns | 0.8716 |
| IMPG2 PE1 vs. CAG | **** | <0.0001 |
| IMPG2 Prom only vs. IMPG2 Prom center short | ns | >0.9999 |
| Enh | | |
| IMPG2 Prom only vs. IMPG1 PE1 | ns | 0.2688 |
| IMPG2 Prom only vs. CAG | **** | <0.0001 |
| IMPG2 Prom center short Enh vs. IMPG1 PE1 | ns | 0.289 |
| IMPG2 Prom center short Enh vs. CAG | **** | <0.0001 |
| IMPG1 PE1 vs. CAG | **** | <0.0001 |

ns: non-significant. P: promoter. E: enhancer.

TABLE 14
Statistical Results in FIG. 31C.

| | | Adjusted |
|---|---|---|
| Tukey's multiple comparisons test | Summary | P Value |
| IMPG2 PE1 vs. IMPG2 Prom only | ns | 0.9994 |
| IMPG2 PE1 vs. IMPG2 Prom center short Enh | ns | 0.9995 |
| IMPG2 PE1 vs. IMPG1 PE1 | ns | 0.9997 |
| IMPG2 PE1 vs. CAG | **** | <0.0001 |
| IMPG2 Prom only vs. IMPG2 Prom center short | ns | >0.9999 |
| Enh | | |
| IMPG2 Prom only vs. IMPG1 PE1 | ns | 0.9937 |
| IMPG2 Prom only vs. CAG | **** | <0.0001 |
| IMPG2 Prom center short Enh vs. IMPG1 PE1 | ns | 0.9943 |
| IMPG2 Prom center short Enh vs. CAG | **** | <0.0001 |
| IMPG1 PE1 vs. CAG | **** | <0.0001 |

ns: non-significant. P: promoter. E: enhancer.

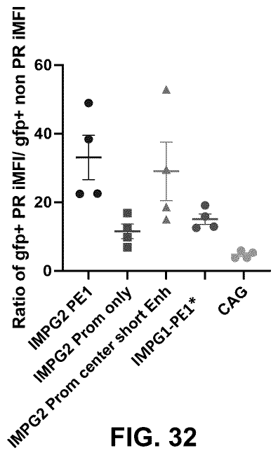

TABLE 15
Statistical Results in FIG. 32.

| | | Adjusted |
|---|---|---|
| Tukey's multiple comparisons test | Summary | P Value |
| IMPG2 PE1 vs. IMPG2 Prom only | ns | 0.05 |
| IMPG2 PE1 vs. IMPG2 Prom center short Enh | ns | 0.976 |
| IMPG2 PE1 vs. IMPG1 PE1 | ns | 0.1253 |
| IMPG2 PE1 vs. CAG | ** | 0.0077 |
| IMPG2 Prom only vs. IMPG2 Prom center short | ns | 0.1411 |
| Enh | | |
| IMPG2 Prom only vs. IMPG1 PE1 | ns | 0.9849 |
| IMPG2 Prom only vs. CAG | ns | 0.8635 |
| IMPG2 Prom center short Enh vs. IMPG1 PE1 | ns | 0.3139 |
| IMPG2 Prom center short Enh vs. CAG | * | 0.0238 |
| IMPG1 PE1 vs. CAG | ns | 0.5877 |

ns: non-significant. P: promoter. E: enhancer.

We claim:

1. A genetically engineered promoter-enhancer construct comprising a promoter and at least one enhancer operatively linked to the promoter that specifically promotes expression in a photoreceptor cell of a nucleic acid that is operatively linked to the promoter-enhancer construct and capable of being expressed therein.

2. The genetically engineered promoter-enhancer construct of claim 1, wherein the promoter-enhancer is positioned upstream of the nucleic acid.

3. The genetically engineered promoter-enhancer construct of claim 1, wherein the enhancer is derived from a human interphotoreceptor matrix proteoglycan (IMPG) gene.

4. The genetically engineered promoter-enhancer construct of claim 3, wherein the enhancer has a nucleotide sequence that is at least 90% identical to SEQ ID NO: 1, SEQ ID NO: 6, or SEQ ID NO: 17.

5. The genetically engineered promoter-enhancer construct of claim 3, wherein the enhancer has a nucleotide sequence that is at least 90% identical to SEQ ID NO: 25.

6. The genetically engineered promoter-enhancer construct of claim 1, wherein the promoter is derived from a human interphotoreceptor matrix proteoglycan (IMPG) gene.

7. The genetically engineered promoter-enhancer construct of claim 6, wherein the promoter has a nucleotide sequence that is at least 90% identical to SEQ ID NO: 2, SEQ ID NO: 4, or SEQ ID NO: 16.

8. The genetically engineered promoter-enhancer construct of claim 6, wherein the promoter has a nucleotide sequence that is at least 90% identical to SEQ ID NO: 26.

9. The genetically engineered promoter-enhancer construct of claim 1, having a nucleotide sequence that is 90% identical to SEQ ID NO: 3, SEQ ID NO: 7, SEQ ID NO: 8, SEQ ID NO: 9, SEQ ID NO: 18, SEQ ID NO: 19, or SEQ ID NO: 20.

10. The genetically engineered promoter-enhancer construct of claim 1, having a nucleotide sequence that is 90% identical to SEQ ID NO: 27.

11. The genetically engineered promoter-enhancer construct of any one of claims 1-8 wherein the enhancer is positioned upstream from the promoter.

12. The genetically engineered promoter-enhancer construct of any one of claims 1-8, wherein the enhancer is positioned downstream from the promoter.

13. A recombinant expression construct capable of expressing a gene or a gene expression modulating sequence in a photoreceptor cell comprising the promoter-enhancer construct of claims 1-12, operatively linked to a nucleic acid encoding the gene or the gene expression modulating sequence.

14. The recombinant expression construct of claim 13 further comprising a vector.

15. A method of expressing the gene or the gene expression modulating sequence in the photoreceptor cell comprising introducing into the cell the recombinant expression construct of claim 13 or 14.

16. A method of modulating expression of a target gene in the photoreceptor cell comprising introducing into the cell a recombinant expression construct of claim 13 or 14.

17. The method of claim 16, wherein the gene expression modulating sequence is an siRNA complementary to the target gene and the expression of which in the photoreceptor cell is modulated thereby.

18. The method of claim 16, wherein the recombinant expression construct encodes components of a gene editing system, wherein expression of the target gene in the photoreceptor cell is modulated thereby.

19. The method of claim 16, wherein the expression of the encoded gene corrects a genetic mutation on the target gene in the photoreceptor cell.

20. A pharmaceutical composition comprising the recombinant expression construct of claim 13 or claim 14 and a pharmaceutically acceptable carrier.

21. A method of treating a retinal disease in a subject comprising administering the pharmaceutical composition of claim 20 to the subject.

22. The method of claim 21, wherein the disease is a photoreceptor-specific or inherited retinal disease.

23. The method of claim 22, wherein the disease is retinitis pigmentosa, Leber congenital amaurosis, Usher syndrome, Stargardt disease, age-related or inherited macular degeneration.

24. The method of any one of claims 21-23, wherein the administration is subretinal injection.
